(19) United States
(12) Patent Application Publication
(10) Pub. No.: US 2013/0288254 A1
(43) Pub. Date: Oct. 31, 2013

(54) Droplet Actuator and Droplet-Based Techniques

(71) Applicants: Michael G. Pollack, Durham, NC (US); Vijay Srinivasan, Durham, NC (US); Allen E. Eckhardt, Durham, NC (US); Prasanna Thwar, Los Altos, CA (US); Zhishan Hua, Cary, NC (US); Arjun Sudarsan, Cary, NC (US); Theodore Winger, Morrisville, NC (US)

(72) Inventors: Michael G. Pollack, Durham, NC (US); Vijay Srinivasan, Durham, NC (US); Allen E. Eckhardt, Durham, NC (US); Prasanna Thwar, Los Altos, CA (US); Zhishan Hua, Cary, NC (US); Arjun Sudarsan, Cary, NC (US); Theodore Winger, Morrisville, NC (US)

(73) Assignee: ADVANCED LIQUID LOGIC, INC., Morrisville, NC (US)

(21) Appl. No.: 13/390,121

(22) PCT Filed: Aug. 13, 2010
(86) PCT No.: PCT/US2010/045461

§ 371 Date: Feb. 13, 2012

Related U.S. Application Data
(60) Provisional application No. 61/233,638, filed on Aug. 13, 2009.
(60) Provisional application No. 61/238,486, filed on Aug. 31, 2009.
(60) Provisional application No. 61/241,488, filed on Sep. 11, 2009.
(60) Provisional application No. 61/260,220, filed on Nov. 11, 2009.
(60) Provisional application No. 61/291,108, filed on Dec. 30, 2009.
(60) Provisional application No. 61/321,259, filed on Apr. 6, 2010.
(60) Provisional application No. 61/364,528, filed on Jul. 15, 2010.

(51) Int. Cl. C12Q 1/68

(52) U.S. Cl. CPC C12Q 1/686
USPC 435/6.12

(57) ABSTRACT

The invention is directed to certain droplet actuated molecular techniques. In one embodiment, the invention provides droplet actuator methods for detection of single nucleotide polymorphisms (SNPs) in a DNA sequence using digital microfluidics, including droplet actuator-based sample preparation and SNP analysis. In another embodiment, the invention provides droplet actuator devices and methods for providing integrated sample preparation and multiplexed detection of an infectious agent, such as HIV. In yet another embodiment, the invention provides droplet actuator devices and techniques for PCR amplification and detection of specific nucleic acid sequences using digital microfluidics, including droplet actuator-based sample preparation and target nucleic acid analysis. In yet another embodiment the invention provides methods for performing hot-start PCR on a droplet actuator. In yet another embodiment, the method of the invention combines PCR amplification with pyrosequencing to investigate specific sequences.

### 1 RELATED APPLICATIONS

In addition to the patent applications cited herein, each of which is incorporated herein by reference, this patent application is related to and claims priority to U.S. Provisional Patent Application Nos. 61/233,638, filed on Aug. 13, 2009, entitled “Restriction Endonuclease Detection of SNPs by Digital Microfiuidics”, 61/238,486, filed on Aug. 31, 2009, entitled “Integrated Sample Preparation and Analysis on a Droplet Actuator”; 61/241,488, filed on Sep. 11, 2009, entitled “Hot-Start PCR on a Droplet Actuator”; 61/260,220, filed on Nov. 11, 2009, entitled “Molecular Techniques for Digital Microfluidics”; 61/291,108, filed on Dec. 30, 2009, entitled “Sample Preparation and Analysis on a Droplet Actuator”; 61/321,259, filed on Apr. 6, 2010, entitled “Molecular Techniques for Digital Microfluidics”; 61/364,528, filed on Jul. 15, 2010, entitled “Molecular Techniques for Digital”; the entire disclosures of which are incorporated herein by reference.

### 2 FIELD OF THE INVENTION

The invention generally relates to droplet actuators and techniques. In particular, the invention is directed to droplet actuator and droplet actuated molecular techniques.

### 3 BACKGROUND

Droplet actuators are used to conduct a wide variety of droplet operations. A droplet actuator typically includes one or more substrates configured to form a surface or gap for conducting droplet operations. The one or more substrates include electrodes for conducting the droplet operations. Droplet operations may be electrode mediated, e.g., electrowetting mediated or dielectrophoresis mediated or Coulombic force mediated. The gap between the substrates, commonly referred to as a droplet operations gap, is typically filled or coated with a filler fluid that is immiscible with the liquid that is to be subjected to droplet operations. There is a need in the art for devices and techniques that facilitate the use of droplet actuators for performing assays using nucleic acids. For example, there is a need for improved methods for implementing SNP genotyping technologies that provides for flexibility in assay design and facilitates diagnosis and/or treatment decisions. In another example, there is a need for techniques that make use of a droplet actuator for combined amplification and detection of specific nucleotide sequences.

Further, there is a need for improved techniques and devices facilitating use of droplet actuators for conducting molecular diagnostic assays, such as immunoassays and quantitative polymerase chain reaction (qPCR) assays.

### 4 SUMMARY OF THE INVENTION

The invention is directed to certain droplet actuated molecular techniques.

In one example, the invention provides a method of detecting a target nucleic acid sequence in a sample nucleic acid. The method may include providing a sample droplet including the sample nucleic acid and reagents sufficient for amplification, the reagents including a primer which produces or can be made to produce a detectable signal; amplifying the nucleic acid in the sample droplet to yield amplicons including the detectable primer; capturing the amplicons on a substrate in the sample droplet; subjecting the amplicons to restriction in the sample droplet; splitting the sample droplet to yield a supernatant droplet and a droplet comprising the substrate; and sensing to detect the detectable signal in the supernatant droplet and/or the droplet including the substrate.

In another example, the invention provides a method of detecting a target nucleic acid sequence in a sample nucleic acid. The method may include providing a droplet including the sample nucleic acid and reagents sufficient for amplification, the reagents including a primer which produces or may be made to produce a detectable signal; amplifying the nucleic acid in the droplet to yield amplicons including the detectable primer; capturing the amplicons on a substrate in the droplet; subjecting the amplicons to restriction in the droplet; aggregating the substrate within a region of the droplet; and sensing to detect the detectable signal in the region of aggregation and/or in a region of the droplet in which the beads are not aggregated.

In yet another example, the invention provides a method of detecting a signal in a droplet. The method may include providing a droplet including a reagent producing a detectable signal; aggregating the reagent in a region of the droplet; and sensing to detect the detectable signal in the region of aggregation and/or in a region of the droplet in which the reagent is not aggregated.

In yet another example, the invention provides a method of detecting single nucleotide polymorphisms (SNPs). The method may include providing a droplet actuator including one or more substrates configured to form a droplet operations gap, the one or more substrates comprising electrodes configured for conducting droplet operations in the gap; a sample reservoir for containing a sample fluid and arranged for dispensing separated sample onto the one or more substrates for transporting the separated sample along the electrodes for processing or analysis, the sample reservoir having magnetic capture beads coated with a substance attractive to a component in the sample; and a magnet movable away from and into proximity to the sample reservoir. The method may further include, adding MRSA DNA to a quantity of a cell lysis solution containing charge switch DNA capture heads; concentrating the beads in a solution off the droplet actuator, and transferring the concentrated head solution to the sample reservoir; dispensing a droplet from the reservoir containing substantially all the beads from the quantity of cell lysis solution; transporting the bead containing droplet away from the reservoir and washing the beads; dispensing and mixing purified DNA from the washed droplet with a PCR mix; conducting a PCR reaction; and detecting target DNA resulting from the reaction.

In yet another example, the invention provides a method of dispensing a separated blood sample from a whole blood sample. The method may include providing a droplet actuator including one or more substrates configured to form a droplet operations gap, the one or more substrates including electrodes configured for conducting droplet operations in the gap; a sample reservoir for containing a sample fluid and arranged for dispensing separated sample onto the one or more substrates for transporting the separated sample along the electrodes for processing or analysis, the sample reservoir containing magnetic capture beads coated with a substance attractive to a component in the sample; and a magnet movable away from and into proximity to the sample reservoir. The method may further include, loading a quantity of whole blood sample into the sample reservoir and incubating the sample to allow for formation of capture antibody-blood cell complexes on the magnetic capture beads; moving the magnet into proximity of the sample reservoir to separate the blood cell complexes from the sample to form a separated sample; and dispensing the separated sample from the sample reservoir onto the electrodes for further processing.

In yet another example, the invention provides a method of preparing a sample and an assay from the sample. The method may include providing a droplet actuator including, one or more substrates configured to form a droplet operations gap, the one or more substrates including electrodes configured for conducting droplet operations in the gap; a sample reservoir for containing a sample fluid and arranged for dispensing separated sample onto the one or more substrates for transporting the separated sample along the electrodes for processing or analysis, the sample reservoir having magnetic capture beads coated with a substance attractive to a component in the sample; and a magnet movable away from and into proximity to the sample reservoir. The method may further include, loading a sample into the sample reservoir on the droplet actuator, and separating a subpopulation in the sample for further processing; dispensing the subpopulation in droplets onto the droplet operations electrodes; conducting at least one immunoassay with the dispensed subpopulation droplets; and detecting at least one property of the resultant immunoassay conducted with the subpopulation droplets.

In yet another example, the invention provides a method of detecting methicillin resistant Staphylococcus aureus (MRSA). The method may include providing a droplet actuator including, one or more substrates configured to form a droplet operations gap, the one or more substrates comprising electrodes configured for conducting droplet operations in the gap; a sample reservoir for containing a sample fluid and arranged for dispensing separated sample onto the one or more substrates for transporting the separated sample along the electrodes for processing or analysis, the sample reservoir containing magnetic capture beads coated with a substance attractive to a component in the sample; and a magnet movable away from and into proximity to the sample reservoir. The method may further include adding MRSA DNA to a quantity of a cell lysis solution containing charge switch DNA capture beads; concentrating the beads in a solution off the droplet actuator, and transferring the concentrated bead solution to the sample reservoir; dispensing a droplet from the reservoir containing substantially all the beads from the quantity of cell lysis solution; transporting the bead containing droplet away from the reservoir and washing the droplet; dispensing and mixing purified DNA from the washed droplet with a PCR mix; conducting a PCR reaction; and detecting target DNA resulting from the reaction.

In yet another example, the invention provides a method of dispensing a sample. The method may include providing a droplet actuator including one or more substrates configured to form a droplet operations gap, the one or more substrates comprising electrodes configured for conducting droplet operations in the gap; a sample reservoir for containing a sample fluid and arranged for dispensing separated sample onto the one or more substrates for transporting the separated sample along the electrodes for processing or analysis, the sample reservoir having magnetic capture beads coated with a substance attractive to a component in the sample; and a magnet movable away from and into proximity to the sample reservoir. The method may further include loading a quantity of sample into the sample reservoir, the sample including a substance aggregatable by formation of antibody-substance cell complexes on the magnetic capture beads and incubating the sample to allow for formation of capture antibody-substance complexes on the magnetic capture beads; moving the magnet into proximity of the sample reservoir to separate the blood cell complexes from the sample to form a supernatant sample, having a reduced content of the aggregatable substance; and dispensing the supernatant sample from the sample reservoir onto the electrodes for further processing.

In yet another example, the invention provides a method of concentrating a sample. The method may include dispensing sample droplets on one or more substrates of a droplet actuator; combining sample droplet with a reaction droplet that contains magnetically responsive capture beads and incubating the combined sample and reaction droplet; and combining successive sample droplets with the reaction droplet and incubating the combined droplet when an assay reports a value below a defined linear range of the assay.

In yet another example, the invention provides a method of dilluting a sample. The method may include dispensing a sample droplet on one or more substrates of a droplet actuator; conducting at least one assay with the dispensed sample droplet; combining the sample droplet with a diluent when an assay reports a value above a defined linear range of the assay; reanalyzing the at least one assay; and repeating as necessary until the assay reports a value within the defined linear range of the assay.

In yet another example, the invention provides a method of amplifying a nucleic acid molecule. The method may include providing a droplet actuator including, one or more substrates arranged to form a substantially enclosed droplet operations gap; electrodes configured for conducting droplet operations in the droplet operations gap; and one or more filler fluids substantially filling the droplet operations gap. The method may further include providing in the droplet operations gap, which is substantially surrounded by the filler fluids, a first droplet, which may include a nucleic acid template; and reagents for amplifying the nucleic acid template; and a second droplet including an enzyme required for amplifying the template. The method may further include providing the first droplet in a thermal zone of the droplet operations gap at an elevated temperature and using droplet operations mediated by the electrodes to combine the first droplet and second droplet to yield an amplification-ready reaction droplet; and conducting a thermal cycling protocol using the reaction droplet.

In yet another example, the invention provides a method of amplifying a nucleic acid molecule. The method may include providing a droplet actuator including one or more substrates arranged to form a substantially enclosed droplet operations gap; electrodes configured for conducting droplet operations in the droplet operations gap; and one or more filler fluids substantially filling the droplet operations gap. The method may further include providing in the droplet operations gap, substantially surrounded by the one or more filler fluids, one or more droplets potentially comprising a nucleic acid template for amplification and comprising reagents for conducting said amplification, wherein one or more enzymes is inactive; activating the enzyme to yield an amplification-ready reaction droplet; and using droplet operations mediated by the electrodes, conducting a thermal cycling protocol using the reaction droplet.

In yet another example, the invention provides a method of performing hot-start PCR. The method may include providing a sample droplet including a target nucleic acid on a droplet actuator; combining the sample droplet with a first reagent droplet to form a reaction droplet; incubating the reaction droplet within a first thermal zone at an elevated temperature; combining the reaction droplet with a second reagent of including an amplification enzyme to form an amplification-ready reaction droplet; incubating the amplification-ready reaction droplet within the first thermal zone; transporting the amplification-ready reaction droplet to a second thermal zone and incubating the amplification-ready reaction droplet within the second thermal zone; and conducting a thermal cycling protocol using the amplification-ready reaction droplet to achieve a desired level of amplification.

In yet another example, the invention provides a method of optimizing real-time PCR. The method may include providing a digital microfluidics system comprising at least one signal monitor; providing a sample droplet comprising a detectable signal component; conducting a PCR reaction; monitoring intensity of the detectable signal component during each thermal cycle; and initiating a next thermal cycle when the signaling component of a sample reaches a plateau.

In yet another example, the invention provides a droplet actuator device. The droplet actuator device may include one or more substrates configured to form a droplet operations gap, the one or more substrates including electrodes configured for conducting droplet operations in the gap; a sample reservoir for containing a sample fluid and arranged for dispensing one or more sample droplets onto the one or more substrates for transporting the one or more sample droplets along the electrodes for processing or analysis; a sensor for sensing a signal from the one or more sample droplets positioned in proximity to at least one of the electrodes; and a magnet positioned such that when one of the one or more sample droplets is positioned at the electrode in proximity to the sensor, any magnetically responsive beads in the sample droplet are concentrated in proximity to the magnet, such that the sensing of the signal by the sensor can be effected without substantial interference from the magnetically responsive beads.

### 5 DEFINITIONS

As used herein, the following terms have the meanings indicated.

“Activate,” with reference to one or more electrodes, means effecting a change in the electrical state of the one or more electrodes which, in the presence of a droplet, results in a droplet operation.

“Bead,” with respect to heads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator or otherwise configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles DYNABEADS® particles, available from Dynal Bead Based Separations (Invitrogen Group), Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule (ligand). The ligand may, for example, be an antibody, protein or antigen, DNA/RNA probe or any other molecule with an affinity for the desired target. Examples of droplet actuator techniques for immobilizing magnetically responsive beads and/or non-magnetically responsive beads and/or concluding droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; international Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by filler fluid. For example, a droplet may be completely surrounded by filler fluid or may be bounded by filler fluid and one or more surfaces of the droplet actuator. As another example, a droplet may be bounded by filler fluid, one or more surfaces of the droplet actuator, and the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000; Kim et al., U.S. patent application Ser. Nos. 10/343,261, entitled “Electrowetting-driven Micropumping,” filed on Jan. 27, 2003, 11/275,668, entitled “Method and Apparatus for Promoting the Complete Transfer of Liquid Drops from a Nozzle,” filed on Jan. 23, 2006; 11/460,188, entitled “Small Object Moving on Printed Circuit Board,” filed on Jan. 23, 2006, 12/465,935, entitled “Method for Using Magnetic Particles in Droplet Microfluidics,” filed on May 14, 2009; 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Micro fluidic Control via. Electrowetting, for Chemical, Biochemical and Biological Assays and the Like,” issued on Jan. 16, 2007; Becker and Gascoyne et al., U.S. Pat. Nos. 7,641,779, entitled “Method and Apparatus for Programmable fluidic Processing,” issued on Jan. 5, 2010, and 6,977,033, entitled “Method and Apparatus for Programmable fluidic Processing,” issued on Dec. 20, 2005; Deere et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al. U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a gap there between and electrodes associated with the one or more substrates and arranged to conduct one or more droplet operations. The base (or bottom) and top substrates may in some cases be formed as one integral component. Certain droplet actuators will include a base (or bottoms substrate, droplet operations electrodes associated with the substrate, one or more dielectric and/or hydrophobic layers a top the substrate and/or electrodes forming a droplet operations surface, and optionally, a top substrate separated from the droplet operations surface by a gap. Various electrode arrangements are discussed throughout the description of the invention. Where multiple substrates are used, a spacer may be provided between the substrates to determine the height of the droplet operations gap. The gap height may, for example, be from about 5 μM to about 1000 μM, or about 50 μM to about 600 μM, or about 100 μM to about 400 μM, or about 200 μM to about 350 μM, or about 250 μM to about 300 μM, or about 275 μM. The spacer may, for example, be formed of a layer of photolithographically patterned polymer film or may be a part of one or both of the substrates. One or more openings may be provided in the one or more substrates for forming a fluid path through which liquid may be delivered into the droplet operations gap of the droplet actuator. The one or more openings may be aligned for interaction with one or more electrodes, e.g., such that fluid flowed into the gap may be subject to droplet operations mediated by such electrodes. It is sometimes useful for the droplets to be exposed to a reference potential or a ground; the ground may be associated with one or both substrates and/or situated between substrates. Electrodes on substrate may be coupled to electrical contacts on the other substrate by any electrically conductive medium. In one embodiment, a reference electrode on the top substrate is coupled to a reference contact on the bottom substrate, e.g., by a conductive substance (such as an electrically conductive foam, epoxy, or resin) situated between the substrates. In various embodiments, the manipulation of droplets by a droplet actuator may be electrode mediated; e.g., electrowetting mediated or dielectrophoresis mediated or Coulombic force mediated. Examples of other methods of controlling fluid flow that may be used in the droplet actuators of the invention include devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electro osmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments; combinations of two or more of the foregoing techniques may be employed in droplet actuators of the invention. Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and 3M™ NOVEC™ electronic coatings (available from 3M Company, St. Paul, Minn.), and other fluorinated monomers for plasma-enhanced chemical vapor deposition (PECVD). In some cases, the droplet operations surface may include a hydrophobic coating having a thickness ranging from about 10 nM to about 1,000 nM. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Srinivasan et al., U.S. Patent Application No. 61/294,874, entitled “Droplet Actuator with Conductive Ink Ground,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nM, preferably about 50 to about 150 nM, or about 75 to about 125 nM, or about 100 nM. In some cases, the bottom substrate includes a PCB substrate that is coated with a dielectric, such as a polyimide dielectric, which is then coated or otherwise treated to make the droplet operations surface hydrophobic. When the substrate is a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.); NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass) and PARYLENE™ N (available from Parylene Coating Services, Inc., Katy, Tex.); TEFLON® AF; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethyleue; polyester; polyethylene naphthalate; cyclo-olefin copolymer (COC); cyclo-olefin polymer (COP); any other PCB substrate material listed above; black matrix resin; and polypropylene. Certain droplet actuators may be adapted for template preparation and/or pyrosequencing protocols and for application of a specific template preparation and/or pyrosequencing protocols. For example, composition of the filler fluid surfactant doping concentration may be selected for performance with reagents used in the template preparation protocol. Droplet transport voltage and frequency may also be selected for performance with reagents used in a template preparation protocol. Design parameters may be varied, e.g., number and placement of on-chip reservoirs, number of independent electrode connections, size (volume) of different reservoirs, placement of magnets/bead washing zones, electrode size, inter-electrode pitch, and gap height (between top and bottom substrates) may be varied. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF and FLUOROPEL® for dip or spray coating, and other fluorinated monomers for plasma-enhanced chemical vapor deposition (PECVD). Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray Industries, Inc., Japan.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.”

“Filler fluid” means a fluid associated with a droplet operations substrate of a droplet actuator, which fluid is sufficiently immiscible with a droplet phase to render the droplet phase subject to electrode-mediated droplet operations. For example, the gap of a droplet actuator is typically filled with a filler fluid. The filler fluid may, for example, be a low-viscosity oil, such as silicone oil or hexadecane filler fluid. Other examples of filler fluids are provided in international Patent Application No. PCT/US2006/047486, entitled, “Droplet-Based Biochemistry,” filed on Dec. 11, 2006; International Patent Application No. PCT/US2008/072604, entitled “Use of additives for enhancing droplet actuation,” filed on Aug. 8, 2008; and U.S. Patent Publication No. 20080283414, entitled “Electrowetting Devices,” filed on May 17, 2007; the entire disclosures of which are incorporated herein by reference. The filler fluid may fill the entire gap of the droplet actuator or may coat one or more surfaces of the droplet actuator. Filler fluid may be conductive or non-conductive.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a splitting operation on a droplet, yielding one droplet with substantially all of the beads and one droplet substantially lacking in the beads.

“Magnetically responsive” means responsive to a magnetic field, “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in gray of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated in away from a certain region of the magnetic field, in each case where the magnetic field in such region is not capable of substantially attracting any magnetically responsive beads in the droplet. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom” “over,” “under” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6 BRIEF DESCRIPTION OF THE DRAWINGS

FIGS. 1A and 1B illustrate an example of a process of PCR amplification and restriction endonuclease digestion for SNP detection;

FIGS. 2A through 2J illustrate top views of an example of a portion of an electrode arrangement of a droplet actuator and show a process of performing restriction endonuclease detection of SNPs on a droplet actuator;

FIGS. 3A through 3D illustrate top views of an example of a portion of an electrode arrangement of a droplet actuator and show a process of performing an invader assay for SNP detection on a droplet actuator;

FIG. 4 illustrates a top view of an example of a portion of an electrode arrangement of a droplet actuator and shows a process of dispensing and transporting a droplet of whole blood;

FIG. 5 illustrates a side view of an example of a portion of a droplet actuator and shows a process of sample preparation from whole blood;

FIG. 6 illustrates a top view of an example of portion of an electrode arrangement of a droplet actuator and shows a process of extracting and loading plasma onto a droplet actuator using a Whatman separation filter;

FIGS. 7A through 7D illustrate top views of an example of a portion of an electrode arrangement of a droplet actuator and show a process of detecting methicillin-resistant Staphylococcus aureus (MRSA) using digital microfluidics PCR;

FIGS. 8A and 8B illustrate top views of an example of a portion of an electrode arrangement of a droplet actuator and show a process of concentrating or auto-diluting a sample in an immunoassay;

FIG. 9 shows an example of a plot of chemiluminescence data for signal improvement by sample concentration in an immunoassay;

FIG. 10A illustrates a top view of an example of a portion of an electrode arrangement if a droplet actuator and shows a process of performing an on-chip dilution protocol;

FIG. 10B illustrates an example of a plot of the results of an on-chip dilution protocol;

FIG. 11 shows an example of a plot of qRT-PCR data for detection of a RNA transcript by use of digital microfluidics;

FIGS. 12A through 12D illustrate top views of an example of a portion of an electrode arrangement of a droplet actuator and a process of performing a hot-start protocol;

FIGS. 13A through 13D again illustrate top views of the electrode arrangement of FIGS. 12A through 1213 and show a process of performing a hot-start protocol that uses DNA polymerase immobilized on magnetically responsive beads;

FIGS. 14A through 14C again illustrate top views of the electrode arrangement of FIGS. 12A through 12D and show a process of performing a hot-start protocol that includes reconstituting dehydrated PCR reagents;

FIGS. 15A through 15E illustrate top views of an example of a portion of an electrode arrangement of a droplet actuator and show a process detecting an immobilized target sequence on a droplet actuator;

FIGS. 16A through 16D illustrate top views of an example of a portion of an electrode arrangement of a droplet actuator and show a process of detecting an unanchored amplified target sequence on a droplet actuator;

FIG. 17 illustrates an example of a process of allele specific primer extension;

FIGS. 18A and 18B illustrate side views of an example of a portion of a droplet actuator and show a process of integrating sample preparation from a scalp swab on a droplet actuator;

FIGS. 19A through 19G illustrate top views of an example of a portion of an electrode arrangement of a droplet actuator and show a process of preparing a single stranded template for pyrosequencing on a droplet actuator;

FIG. 20 illustrates a top view of an example of a portion of an electrode arrangement of a droplet actuator that is configured for pyrosequencing on a droplet actuator;

FIGS. 21A and 21B show an example of a pyrogram and a histogram, respectively, of on-actuator pyrosequencing results of 17-bp sequenced on a 211-bp long C; albicans DNA template using cyclic nucleotide dispensing;

FIG. 22 illustrates a top view of an example of an electrode arrangement of a droplet actuator that is configured for multiplexed real-time PCR;

FIGS. 23A through 23C show an example of the simulation results of finite element thermal analysis of a PCR reaction;

FIGS. 24A and 24B show an example of a plot of real-time PCR data and a plot of PCR efficiency, respectively, of real-time PCR detection of methicillin resistant Staphylococcus aureus (MRSA) genomic DNA;

FIGS. 25A and 25B show two example plots of fluorescence intensity data for cycles 26 through 30 and cycles 36 through 40, respectively, for a 40 cycle real-time PCR;

FIG. 26 shows an example of a plot of fluorescence intensity data for a comparison of real-time PCR using fixed (2 reactions) and variable cycle (1 reaction) times;

FIG. 27 shows an example of a plot of fluorescence intensity data of a two-plea (MRSA and Mycoplasma) real-time PCR assay of Table 5 performed in parallel on the digital microfluidic PCR platform;

FIGS. 28A through 28D illustrate top views of an example of a portion of an electrode arrangement of a droplet actuator and show a process of concentrating and dispensing magnetically responsive beads onto a droplet actuator;

FIGS. 29A and 29B illustrate top views of an example of a portion of an electrode arrangement of a droplet actuator and show a method of manipulating magnetically responsive beads to improve analyte detection;

FIG. 30 shows an example of a plot of fluorescence intensity data for real-time PCR performed with and without an external magnet positioned in proximity to the detection spot;

FIG. 31 shows an example of a plot of fluorescence intensity data from a real-time PCR analysis of Candida albicans DNA in a simulated clinical sample;

FIG. 32 shows an example of a plot of M; pneumoniae concentration versus mean Ct of simulated clinical samples assayed on a conventional real-time PCR platform;

FIG. 33 shows an example of a plot of M; pneumoniae concentration versus mean Ct of simulated clinical samples assayed on the microfluidic real-time PCR platform;

FIG. 34 illustrates a flow diagram of an example of a microfluidic protocol for detection of known and unknown pathogens on a droplet actuator;

FIG. 35 illustrates a flow diagram of an example of a process of using padlock probe technology for amplification of related nucleic acid sequences;

FIG. 36 illustrates a flow diagram of an example of a simplified PCR matrix description using 20 sub-pools of 100 primers for SMART amplification;

FIG. 37 illustrates a flow diagram of an example of a protocol to increase long read sequencing to 3000 bp in about 1 hour;

FIGS. 38A, 38B and 38C illustrate side views of an example of a portion of a droplet actuator and show a process of integrating sample preparation on a droplet actuator;

FIG. 39 illustrates a top view of an example of a droplet actuator (PCR-C01) that is suitable for use in conducting a pyrosequencing template preparation protocol;

FIG. 40 shows an example of a plot of the fluorescence data of the PCR-C01 samples of Table 13 that were collected from the droplet actuator and pooled together for pyrosequencing;

FIG. 41 shows an example of a histogram on-chip pyrosequencing results of 13-bp sequenced on a 211-bp long C; albicans DNA template;

FIG. 42 illustrates a top view of an example of a droplet actuator (PCR-E01) and shows an example layout of fluid reservoirs for collecting and dispensing fluids for integrated template preparation and pyrosequencing reactions;

FIG. 43 shows an example of a histogram of pyrosequencing results of 17-bp sequenced on a 211-bp long C; albicans DNA using a protocol that integrates template preparation and pyrosequencing on the same droplet actuator;

FIG. 44 shows an example of a histogram of pyrosequencing results of 20-bp sequenced on a 211-bp long C; albicans DNA using a protocol that integrates PCR, template preparation, and pyrosequencing on the same droplet actuator;

FIGS. 45A through 45C illustrate top views of an example of a portion of an electrode arrangement of a droplet actuator and show a process of bead immobilization using magnetic forces that are provided directly inside a droplet;

FIGS. 46A and 46B illustrate top views of an example of a portion of an electrode arrangement of a droplet actuator and show examples of head washing by filtration;

FIGS. 47A through 47C illustrate side views of an example of a portion of a droplet actuator and show examples of applying two different magnetic field strengths for bead washing; and

FIG. 48 illustrates a side view of an example of a portion of a droplet actuator and shows an example of using multiple magnets for bead concentration.

### 7 DESCRIPTION

The invention provides droplet actuator devices for and methods of facilitating certain droplet actuated molecular techniques. In one example, the invention provides droplet actuator devices and methods for detection of single nucleotide polymorphisms (SNPs) in a DNA sequence using digital microfluidics. The method of the invention uses polymerase chain reaction (PCR) amplification and restriction endonuclease cleavage to detect the presence or absence of a specific nucleotide in a DNA sequence. The droplet actuator device uses a small sample volume and provides for rapid and accurate detection of SNPs. In various embodiments, the invention also provides for droplet actuator-based sample preparation and SNP analysis. In one embodiment, the device and methods of the invention may be used for rapid and accurate detection of one or more SNPs associated with a particular disease or risk of developing a disease (i.e., medical diagnostics). In another embodiment, the device and methods of the invention may be used to evaluate risk for adverse drug events that may be associated, for example, with alterations in drug absorption and/or metabolism (i.e., pharmacogenetics). In yet another embodiment, the device and methods of the invention may be used in microbial forensics alit/or epidemiology to track the source of a pathogen (e.g., bacteria, virus).

In another example, the invention provides droplet actuator devices and methods for providing integrated sample preparation and multiplexed detection of an infectious agent, such as HIV. Using digital microfluidics technology, the droplet actuator device and methods of the invention provide the ability to perform sample preparation (e.g., plasma from whole blood) and one or more molecular assays, such as multiplexed immunoassays and qRT-PCR from a single blood sample on the same droplet actuator. The droplet actuator device uses a small sample volume (e.g., about 100 to about 200 μL) and provides for rapid and accurate detection of antibodies against HIV proteins and viral RNA. The integrated method of the invention combines two independent test methods (i.e., immunoassays and qRT-PCR) and provides both screening/diagnosis and confirmatory testing using a single blood sample.

In yet another example, the invention provides droplet actuator devices and techniques for PCR amplification and detection of specific nucleic acid sequences using digital microfluidics. The methods of the invention generally involve combining the necessary reactants to form a PCR-ready droplet and thermal cycling the droplet at temperatures sufficient to result in amplification of a target nucleic acid. A droplet including the amplified target nucleic acid may then be transported into a subsequent process, such as a detection process. The droplet actuator device uses a small sample volume and provides for rapid and accurate amplification and detection of target nucleic acid sequences. In various embodiments, the invention also provides for droplet actuator-based sample preparation and target nucleic acid analysis. Combining amplification and detection steps on a droplet actuator provides for rapid and flexible investigation of DNA sequences. In one embodiment, the invention provides methods for performing hot-start PCR on a droplet actuator. Hot-start PCR is typically used to reduce non-specific amplification during the initial net up stages of a PCR assay. In another embodiment, the method of the invention combines PCR amplification with various sequence specific detection technologies for amplified DNA. In yet another embodiment, the method of the invention combines PCR amplification with pyrosequencing to investigate specific sequences.

7.1 Restriction Endonuclease Detection of SNPs by Digital Microfluidics

The invention provides a droplet actuator device and methods for detection of single nucleotide polymorphisms (SNPs) in a DNA sequence using digital microfluidics. The method of the invention uses PCR amplification and restriction endonuclease cleavage to detect the presence or absence of a specific nucleotide in a DNA sequence. The droplet actuator device uses a small sample volume and provides for rapid and accurate detection of SNPs. In various embodiments, the invention also provides for droplet actuator-based sample preparation and SNP analysis.

One SNP genotyping method commonly used combines PCR amplification of a SNP region of interest with detection of restriction fragment polymorphisms (RFLPs), i.e., PCR-RFLP genotyping. SNP genotyping on a droplet actuator provides for flexibility in assay design and facilitates diagnosis and/or treatment decisions.

In one embodiment, the device and methods of the invention may be used for rapid and accurate detection of one or more SNPs associated with a particular disease or risk of developing a disease (i.e., medical diagnostics). In one example, detection of a single nucleotide change may be used to aid in the diagnosis and/or identification of a disease and/or carrier state, such as sickle cell anemia. In another example, multiplexed SNP detection assays for two or more genes and multiple alleles may be used to evaluate the risk of developing complex diseases, such as ischemic heart disease.

In another embodiment, the device and methods of the invention may be used to evaluate risk for adverse drug events that may be associated, for example, with alterations in drug absorption and/or metabolism (i.e., pharmacogenetics).

In yet another embodiment, the device and methods of the invention may be used in microbial forensics and/or epidemiology to track the source of a pathogen (e.g., bacteria, virus). Polymorphisms among isolates or strains may provide information as to the origin, phylogenetic relationships or transmission patterns of those isolates. Examples include, but are not limited to, viruses such as influenza or enteroviruses, antibiotic resistant bacteria such methicillin resistant Staphylococcus aureus (MRSA), biothreats such as anthrax, and linking SNP to virulence or antibiotic resistance of an infectious agent.

In yet another embodiment, the device and methods of the invention lay be used to identify and characterize novel microbial or cellular enzymes that recognize and cleave distinct nucleic acid sequences and/or structures (e.g., hairpins, mismatched DNA sequences, repeated sequences). Novel site-specific enzymes may be useful in further characterizing allele specific polymorphisms.

7.1.1 Detection of SNPs on a Droplet Actuator

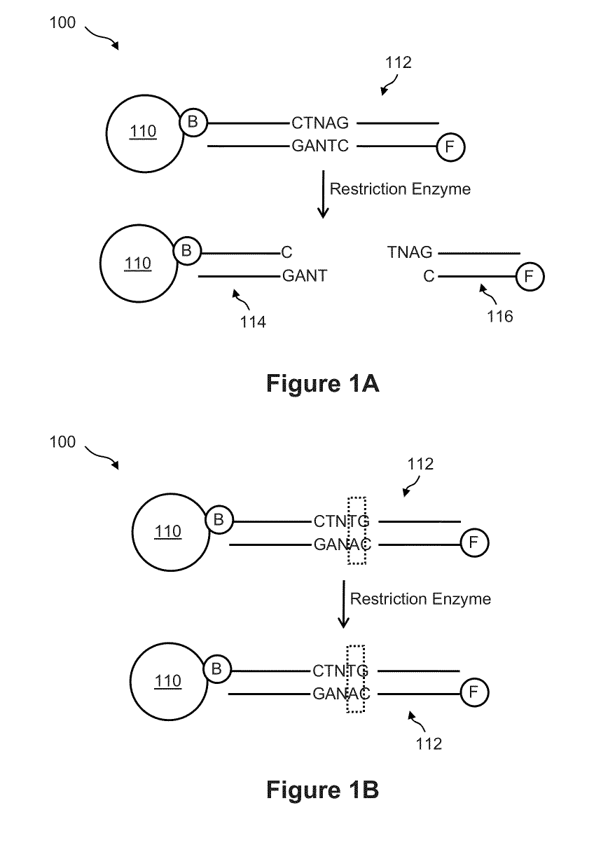

FIGS. 1A and 1B illustrate an example of a process 100 of PCR amplification and restriction endonuclease digestion for SNP detection. In this embodiment, a SNP region of interest is amplified, fluorescently labeled and anchored to magnetically responsive beads. Restriction endonuclease digestion is then used to interrogate the SNP amplicon. In one example, a SNP region may be amplified by PCR using a biotinylated primer (B) and a fluorescently labeled primer (F) to yield a biotinylated and fluorescently labeled amplicon 112. Amplicon 112 may be anchored on magnetically responsive beads 110 that are coated with streptavadin through formation of a biotin-streptavidin complex. Restriction endonuclease digestion may then be used to distinguish between a normal allele and a SNP allele. In one example, a normal allele includes a DNA sequence (e.g., CTNAG) that is recognized by a specific restriction endonuclease Dde1, FIG. 1A). A SNP allele includes a DNA sequence that includes a single nucleotide change (e.g., A to T, indicated by boxed region) that eliminates the restriction endonuclease cleavage site (FIG. 1B). As shown in FIG. 1A, digestion of an anchored amplicon 112 generated from a normal allele with a restriction endonuclease results in two fragments: a biotinylated and anchored amplicon fragment 114 and a fluorescent amplicon fragment 116 that is no longer attached to beads 110. As shown in FIG. 1B, if the restriction sequence is not present in amplicon 112 (i.e., a SNP allele), no cleavage occurs and the fluorescence remains associated with the beads 110.

In another example, a SNP allele may include a DNA sequence that has a single nucleotide change that is recognized by a specific restriction endonuclease and a corresponding normal allele that is not recognized by the restriction endonuclease.

The heterozygosity or homozygosity of a DNA sample may also be determined. For example, in homozygous samples, all of the labeled DNA may be cleaved removing substantially all the fluorescence from the beads. Alternatively, none of the labeled DNA may be cleaved leaving all the fluorescence associated with the beads. For a heterozygous sample, one half of the fluorescence will remain on the beads and one half of the fluorescence will be removed after restriction enzyme digestion.

In an alternative embodiment, a second reaction step may be used to remove substantially all the DNA from beads 110. For example, a first restriction enzyme may be used to interrogate a SNP (e.g., loss of a restriction site) and the amount of fluorescence released determined. A second reaction step (e.g., a second enzyme digestion or temperature-dependent DNA denaturation) may be used to release substantially all the bound DNA from heads 110. The amount of fluorescence released in the second reaction step may then be compared to the amount of fluorescence released in the first reaction step to determine heterozygosity of a DNA sample.

In an alternative embodiment, DNA may, for example, be labeled during PCR amplification by incorporation of a fluorescent dNTP, such as fluorescently tagged dCTP or dUTP.

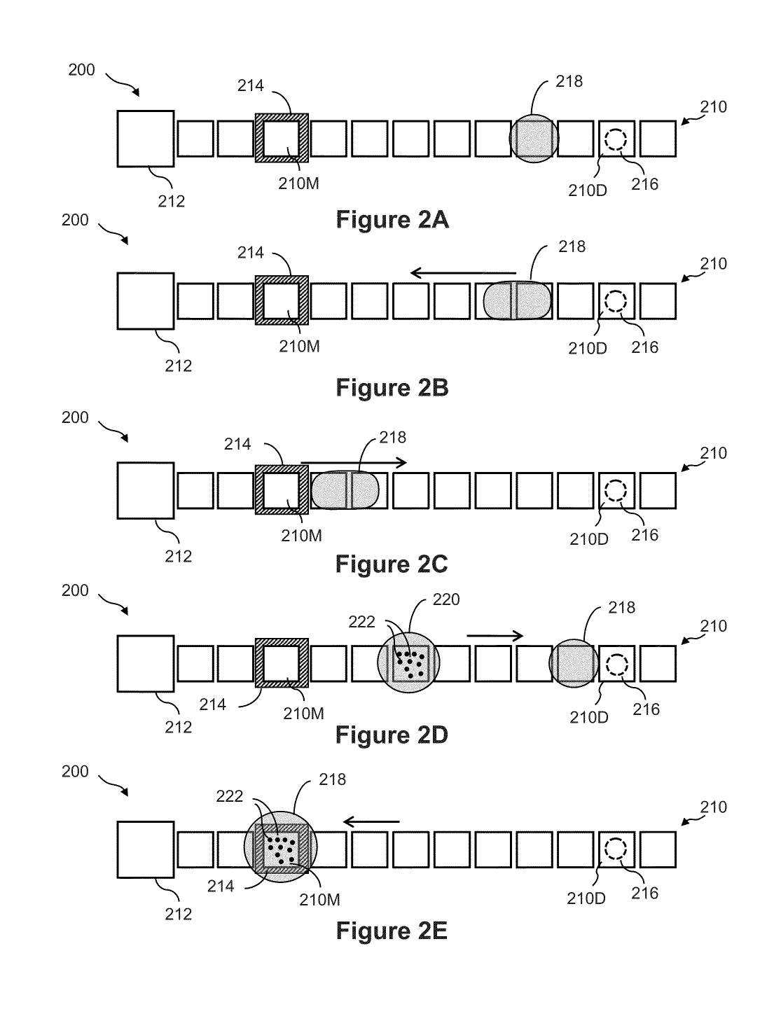

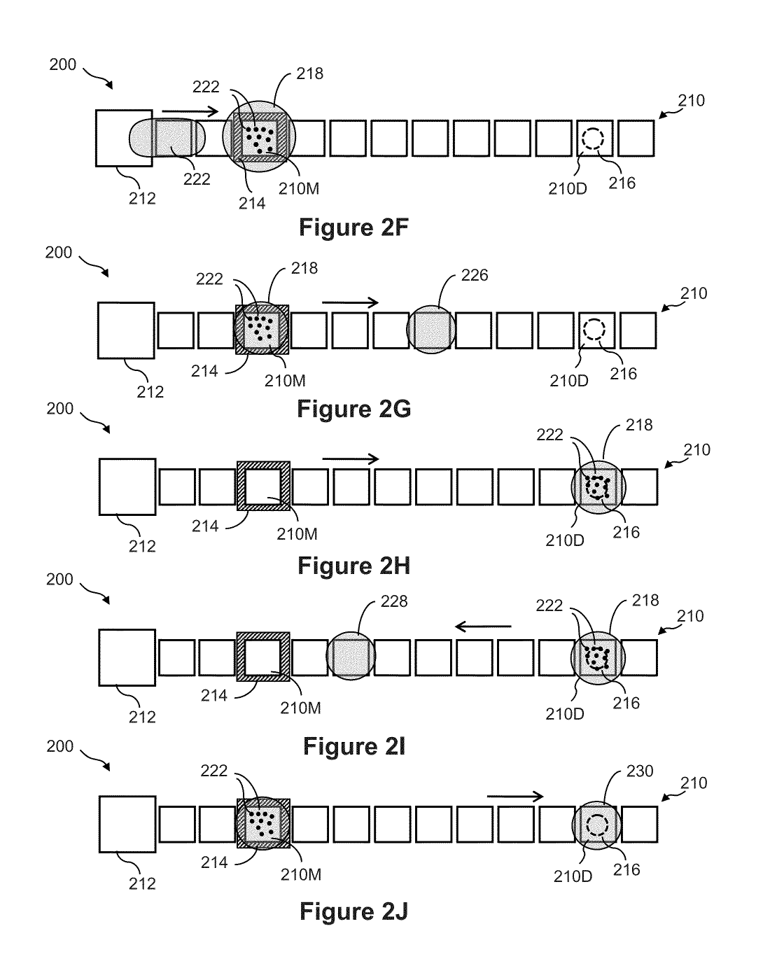

FIGS. 2A through 2J illustrate top views of an example of a portion of an electrode arrangement 200 of a droplet actuator (not shown) and show a process of performing restriction endonuclease detection of SNPs on a droplet actuator. The method of the invention of FIGS. 2A through 2J is an example of a SNP detection protocol wherein target nucleic acids (i.e., SNP regions) are labeled and amplified and immobilized on magnetically responsive beads prior to incubation with an appropriate restriction endonuclease. To quantitate cleavage products, reaction droplets containing the magnetically responsive beads are transported using droplet operations to a detection spot on a droplet actuator. All steps in the SNP detection protocol, including sample and reagent dispensing, incubations, bead washing and detection, are fully automated and under software control.

Electrode arrangement 200 may include an arrangement of droplet operations electrodes 210 (e.g., electrowetting electrodes) and a wash reservoir 212 that is configured for PCR and restriction endonuclease analysis. Droplet operations are conducted atop droplet operations electrodes 210 on a droplet operations surface. A magnet 214 is arranged in close proximity to droplet operations electrodes 210. In particular, magnet 214 is arranged such that certain droplet operations electrodes 210 (e.g., droplet operations electrode 210M) are within the magnetic field thereof. Magnet 214 may, for example, be a permanent magnet or an electromagnet. A detection spot 216 may be arranged in close proximity to droplet operations electrode 210D. A droplet 218 may be positioned at a certain droplet operations electrode. Various droplet operations such as transporting, merging and splitting may be performed on droplet 218. In one embodiment, droplet 218 may, for example, be a sample droplet that includes purified genomic DNA to be evaluated for a SNP of interest.

An example of a process of SNP detection on a droplet actuator may include, but is not limited to, the following steps:

In one step, FIG. 2A shows droplet 218 that is positioned at a certain droplet operations electrode 210. In one example, droplet 218 includes genomic DNA (e.g., purified blood cell DNA) and PCR reagents (e.g., nucleotides, enzyme, and buffers) and primer pairs for amplification of a region encompassing a SNP. One primer may, for example, be a biotinylated primer (e.g., biotinylated forward primer). The second primer may, for example, be a fluorescently labeled primer (e.g., fluorescently labeled reverse primer).

In other steps, FIGS. 2B and 2C show an incubation process, in which droplet 218 is repeatedly transported back and forth (in direction indicated by the arrow) using droplet operations between thermal reaction zones (not shown) for PCR amplification of target DNA. After a sufficient number of thermal cycle reactions, the amount of amplified nucleic acid in the liquid phase of droplet 218 may be of sufficient quantity for detection of a SNP region of interest by restriction endonuclease digestion. The PCR amplicons are biotinylated and fluorescently labeled.

In another step, FIG. 2D shows a reagent droplet 220 that may include a quantity of magnetically responsive beads 222. Magnetically responsive beads 222 are coated with streptavidin. Streptavidin has an affinity for the biotin on the biotinylated PCR amplicons. Reagent droplet 220 is merged with droplet 218 using droplet operations. Merged droplet 218 is repeatedly transported back and forth via droplet operations (not shown) to adjacent droplet operations electrodes 210. Repeated transporting of merged droplet 218 is used during incubation of magnetically responsive beads 222 with the sample in order to provide sufficient resuspension and mixing of magnetically responsive beads 222 for optimal biotin-streptavidin binding. The PCR amplicons are immobilized on magnetically responsive beads 222 through formation of a biotin-streptavidin complex.

In another step, FIG. 2E shows merged droplet 218 that has magnetically responsive beads 222 therein transported using droplet operations to droplet operations electrode 210M (i.e., into the magnetic field of magnet 214).

In other steps, FIGS. 2F and 2G show a bead washing process, in which a wash droplet 224 is transported from wash reservoir 212 along droplet operations electrodes 210 and combined using droplet operations with merged droplet 218, which is retained at droplet operations electrode 210M, to form a merged/wash droplet 218. Magnetically responsive beads 222 are immobilized by the magnetic field of magnet 214.

Merged/wash droplet 218 may be divided using droplet operations into two or more droplets: one or more droplets with beads and one or more droplets without a substantial amount of beads (e.g., supernatant droplet). In one embodiment, the merged/wash droplet is divided using droplet operations into sample droplet 218 that has magnetically responsive beads 222 therein and a supernatant droplet 226 without a substantial amount of beads.

The steps shown in FIGS. 2F and 2G may be repeated multiple times until a sufficient degree of purification is achieved. Each cycle produces a droplet including the beads but with a decreased level of unbound material. In one embodiment, magnetically responsive beads 222 may be released by removing the magnetic field of magnet 214. Removing the magnetic field may be useful to enhance washing by freeing unbound material which may be trapped in the immobilized beads.

In another step, FIG. 2H shows sample droplet 218 that has magnetically responsive beads 222 therein transported using droplet operations to droplet operations electrode 210D, which is within the range of detection spot 216. An imaging device (e.g., a fluorimeter, not shown), arranged in proximity of detection spot 216, is used to capture and quantitate the amount of fluorescence anchored on magnetically responsive beads 222 in sample droplet 218.

In another step, FIG. 2I shows an incubation process in which sample droplet 218 is merged using droplet operations with a reagent droplet 228 that includes a restriction endonuclease that is specific for the SNP region of interest. After a sufficient period of time for restriction endonuclease digest of the DNA sample, sample droplet 218 is washed as described in reference to FIGS. 2F and 2G. In one embodiment, the merged/wash droplet is divided using droplet operations into sample droplet 218 that has magnetically responsive beads 222 therein and a supernatant droplet 230.

In another step. FIG. 2J shows supernatant droplet 230 transported using droplet operations to droplet operations electrode 210D, which is within the range of detection spot 216. An imaging device (e.g., a fluorimeter, not shown), arranged in proximity of detection spot 216, is used to capture and quantitate the amount of fluorescence in supernatant droplet 230. Supernatant droplet 230 may then be transported to a waste reservoir (not shown) and discarded. Subsequently, sample droplet 218 that has magnetically responsive beads 222 therein may also be transported using droplet operations to detection spot 216 and the amount of fluorescence anchored on magnetically responsive beads 222 determined. Referring again to FIGS. 1A and 1B, if the restriction sequence is present, the DNA is cleaved and the fluorescent portion of the DNA is contained in supernatant droplet 230. If the restriction sequence is not present, no cleavage occurs and the fluorescence remains in sample droplet 218 on magnetically responsive beads 222 therein.

In an alternative embodiment, restriction endonuclease cleavage and fluorescence detection may be performed as a single step. For example, the washing protocol of FIGS. 2F and 2G may be eliminated to yield a merged sample/reagent droplet 218. A second magnet (not shown) may be positioned at a distance from droplet operations electrode 210D and merged sample/reagent droplet 218 to provide a sufficient magnet field to gently attract and aggregate magnetically responsive beads 222 to the edge of merged sample/reagent droplet 218 and away from detection spot 216. The strength of the magnetic field provided by the second magnet is such that magnetically responsive beads 222 do not form a tight aggregate and may be easily redistributed in subsequent droplet operations. If the fluorescent signal is associated with magnetically responsive beads 222, a drop in fluorescence signal will be detected as magnetically responsive beads 222 are pulled to the side of merged sample/reagent droplet 218. If the fluorescence signal is not associated with magnetically responsive beads 222 (i.e., removed by restriction enzyme digestion), the signal will remain constant. Measurement of the fluorescent signal may be repeated any number of times during the reaction process.

7.1.1.1 Invader Technology for SNP Genotyping

The INVADER® assay (available from Third Wave Technologies, Inc., Madison. WI) may be used to interrogate SNPs directly from genomic DNA without amplification of the target sequence.

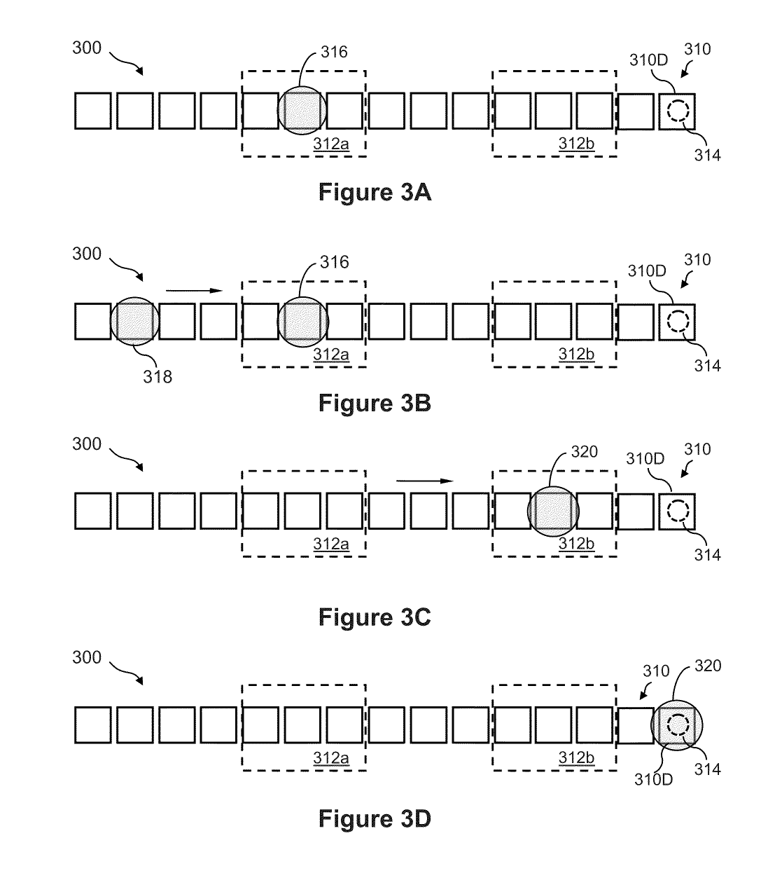

FIGS. 3A through 3D illustrate top views of an example of a portion of an electrode arrangement 300 of a droplet actuator (not shown) and show a process of performing an invader assay for SNP detection on a droplet actuator. Electrode arrangement 300 may include an arrangement of droplet operations electrodes 310 (e.g., electrowetting electrodes). Droplet operations are conducted atop droplet operations electrodes 310 on a droplet operations surface. Two temperature control zones 312, such as temperature control zone 312a and 312b, may be associated with electrode arrangement 300 for performing an invader assay for interrogation of a SNP region of interest. Thermal control elements (not shown) control the temperature of filler fluid (not shown) in the vicinity of temperature control zones 312a and 312b. For example, temperature control zone 312a may be heated to about 95° C. (melting temperature), which is a temperature sufficient for denaturation of DNA template. Temperature control zone 312b may, for example, be heated to about 63° C. to allow cycling of primary probes and INVADER® probes (available from Third Wave Technologies, Inc., Madison, Wis.). While two temperature control zones 312 are shown, any number of temperature control zones 312 may be associated with electrode arrangement 300. A detection spot 314 may be arranged in close proximity to droplet operations electrode 310D.

An example of a process of performing an invader assay for SNP detection on a droplet actuator may include, but is not limited to, the following steps:

In one step, FIG. 3A shows a sample droplet 316 that is positioned at a certain droplet operations electrode 310 within temperature control zone 312a. Sample droplet 316 may, for example, include genomic DNA for SNP interrogation. Because sample droplet 316 is within temperature control zone 312a, the nucleic acid is single-stranded (denatured).

In other steps, FIGS. 3B and 3C show an incubation process in which a reagent droplet 318 is merged using droplet operations with sample droplet 316 within temperature control zone 312a to yield a reaction droplet 320. Reagent droplet 318 includes two primary probes, an INVADER® probe, and two different fluorescent resonance energy transfer (FRET) probes. Reagent droplet 318 also includes an endonuclease (cleavase). A first primary probe is specific for one allele and a second primary probe is specific for the other allele. The alleles are interrogated using FRET probes that correspond to specific primary probes (e.g., FRET probe 1 and primary probe 1; FRET probe 2 and primary probe 2).

Reaction droplet 320 is transported using droplet operations to a certain droplet operations electrode 310 within temperature control zone 312b. Reaction droplet 320 is incubated in temperature control zone 312b for a period of time that is sufficient for probe hybridization and endonuclease cleavage. Cleavase endonuclease recognizes and cleaves the three-dimensional structure that is formed by hybridization of the two overlapping oligonucleotide probes (i.e., primary probe 1, allele 1 and INVADER® probe or primary probe 2, allele 2 and INVADER® probe) to the target sequence. No cleavage occurs in mismatched hybridizations (e.g., primary probe 1, allele 2, and INVADER® probe). Cleavage of the primary probe releases a fragment that anneals to the appropriate FRET probe and initiates a second cleavage reaction that releases the fluorescent dye. The reaction conditions allow cycling of the primary probes and invader probes producing multiple rounds of primary probe cleavage per DNA target.

In another step, FIG. 3D shows reaction droplet 320 transported using droplet operations to droplet operations electrode 310D, which is within the range of detection spot 314. An imaging device (e.g., fluorimeter, not shown), arranged in proximity of detection spot 314, is used to capture and quantitate the amount of fluorescence in reaction droplet 320 from the two different FRET probes. Multiple fluorescent signals are produced per target.

7.1.2 Preparation of Genomic DNA on a Droplet Actuator

In another embodiment, genomic DNA from a biological sample may be prepared on the droplet actuator. Genomic DNA, such as genomic DNA from blood cells, may be prepared using, for example, DYNABEADS® DNA direct (available from Dynal Bead Based Separations (Invitrogen Group), Carlsbad, Calif.). A droplet including lysis buffer and magnetically responsive DYNABEADS® beads may be combined using droplet operations with a blood sample to yield a lysed sample droplet in which released DNA is hound to the DYNABEADS® heads. The DNA capture droplet may be transported using droplet operations into the presence of a magnet and washed using a merge-and-split wash protocol to remove unbound material, yielding a washed bead-containing droplet substantially lacking in unbound material. A droplet including resuspension buffer may be merged with the washed bead-containing droplet, yielding a DNA/bead-containing droplet. The DINA/bead-containing droplet may be transported using droplet operations into a thermal zone to promote release of DNA from the DYNABEADS® beads, e.g., by heating to approximately 65° C. The eluted DNA contained in the droplet surrounding the DYNABEADS® beads may then be transported away from the DYNABEADS® heads for further processing, e.g., for execution of a droplet based PCR amplification protocol and restriction endonuclease detection of a SNP region of interest.

7.1.3 Examples of Restriction Endonuclease-Based SNP Genotyping

7.1.3.1 Medical Diagnostics and Pharmacogenetics

Because of the flexibility and programmability of the digital microfluidics platform, multiplexed assays for two or more genes and/or alleles may be readily performed, in addition, two or more different types of assays, such as PCR, restriction endonuclease cleavage, and allele-specific PCR may be readily performed sequentially and/or simultaneously on a droplet actuator.

Examples of PCR-RFLP analysis for a specific disease and/or risk are shown in Table 1. Examples of PCR-RFLP analysis for evaluation of risk for an adverse drug event are shown in Table 2.

TABLE 1
Examples of medical diagnostic applications
of PCR-RFLP analysis

| Disease | Gene | SNP | Enzyme |
|---|---|---|---|
| Sickle Cell Anemia | β-globin¹ | GAG > GTG | DdeI |
| Ischemic Heart | *Apolipoprotein E4 | | |
| Disease | allele (APOE)² | | |
| | E2 allele (position A) | CGC > TGC | HhaI |
| | E3 allele (position A) | CGC > TGC | HhaI |
| | E2 allele (position B) | CGC > TGC | HhaI |
| | APOE promoter region | −1254T > C | AluI |
| | | −1318A > T | DpnII |
| | | −1046G > T | DpnII |
| | Lipoprotein lipase | −93T > G | ApaI |
| | (LPL)^{3,4,5} | Asp9Asn | SalI |
| | | (G > A) | |
| | | Gly188Glu | AvaII |
| | | (GGG > GAG) | |
| Type II Diabetes | Apolipoprotein B | T71I | ApaLI |
| | (APOB)⁶ | A591V | AluI |
| | | L2712P | MvaI |
| | | R3611Q | MspI |
| | | E4154K | EcoRI |

*APOE genotyping has also been used to evaluate Alzheimer's disease
Note:
some restriction digests yield multiple fragments distinguishing different genotypes; appropriate PCR primer design may reduce the complexity of restriction fragments.

TABLE 2
SNP analysis of Thiopurine S-Methyltransferase gene⁷

| SNP | Enzyme |
|---|---|
| 238G > C | Bsl 1 |
| 460G > A | Mwo 1 |
| 719A > G | Acc I |

7.1.3.2 Methicillin Resistant Staphylococcus aureus (MRSA)

Methicillin-resistant Staphylococcus aureus (MRSA) is a significant cause of healthcare- and community-associated infections, and its prevalence continues to increase. High-level resistance to methicillin is caused by the mecA gene, which encodes an alternative penicillin-binding protein. PBP 2a which has low affinity for β-lactam antibiotics. The mecA regulon (mecA, mec1, and mecR1) is carried by a mobile genetic element designated staphylococcal cassette chromosome mec (SCC/mec)^{9, 10, 11}. SCCmec also includes the ccr gene complex (ccrA and ccrB, or ccrC) and J regions (junkyard, J1, J2, and J3). The structural organization of SCCmec is J1-ccr-J2-mec-J3. SCCmec genotypes are defined by the combination of the class of mec gene complex (3 classes) with the ccr allotype (four ccrAB allotypes, ccrC). Six SCCmec types (I-VI) have been identified in S. aureus. Variations in the J regions may be used for defining SCCmec subtypes¹¹.

SCCmec typing has been established as an important component in the characterization and identification of MRSA strains. Currently, increasing numbers of community-acquired MRSA (CA-MRSA) strains are appearing that area able to cause severe infections in otherwise healthy people. CA-MRSA strains are generally SCCmec type IV or type V. Multiplex PCR assays for rapid SCCmec typing have been developed based on sequence variations in the mecA complex and/or the ccr gene complex. In one example, PCR amplification of the ccrB gene and subsequent HinfI and BsmI restriction endonuclease digestion may be used to rapidly identify four SCCmec types (I-IV), especially type IV, based on different RFLP patterns¹⁰. In another example, a single multiplex PCR assay may be used for the rapid identification of all major subtypes of SCCmec type IV^{11, 12}.

7.1.3.3 Influenza A Viruses

Influenza A viruses circulate worldwide and cause annual epidemics of human respiratory illness. Influenza A viruses are classified by subtype on the basis of the two main surface glycoproteins hemagglutinin (HA) and neuraminidase (NA). Different subtypes (e.g., H1N1 and H3N2) may be in circulation among human populations at different times. Influenza A viruses are further characterized into strains. Because influenza viruses are dynamic and constantly evolving, new strains continually appear. In addition, new subtypes of influenza A may be introduced into the human population from animal sources (e.g., avian, swine) or by genetic reassortment i.e., mixing, of human and animal influenza A genes to create a new subtype.

PCR-RFLP methodologies for genotyping influenza A viruses may be based on polymorphisms in HA¹³ and/or NA coding regions or internal viral gene sequences¹⁴. In one example, two H3N2 influenza viral strains. A/LA/1/87 and A/Sydney/5/97 may be distinguished by HpaI digestion of a PCR amplicon¹³. In this example, the A/LA/1/87 PCR amplicon has a single HpaI restriction site that is absent in the A/Sydney/5/97 PCR amplicon.

In another example, a genotyping strategy may include RFLP analysis of one or more internal gene sequences of influenza A viruses. A genotyping strategy for distinguishing H1N1, H3N2, and H5N1 subtypes is shown in Table 3. In this example, conserved primer sites were identified for each of 6 internal influenza A virus genes¹⁴. The sequences of each PCR amplicon were then analyzed to identify a single, unique restriction endonuclease site for each viral subtype. For example, in the viral NS gene amplicon, a single Drat site is unique to the H1N1 viral subtype, a single XbaI site is unique to the H3N2 subtype, and a single BsrBI site is unique to the H5N1 subtype. This genotyping strategy may be readily updated (e.g., PCR primer sequences and/or restriction enzymes) to compensate for changes in viral subtypes in current circulation.

TABLE 3
Genotyping H1N1, H3N2, and H5N1 Influenza A viruses¹⁴

| | |
|---|---|
| Amplicon | Subtype-specific restriction enzyme |

| Gene | size (bp) | H1N1 | H3N2 | H5N1 |
|---|---|---|---|---|
| NS | 890 | DraI | XbaI | BsrBI |
| M | 847 | HindIII | ScaI | AvaII |
| NP | 1506 | HaeII | SacII | BamHI |
| PA | 773 | BbsI | EcoNI | NsiI |
| PB1 | 715 | ScaI | XmnI | BsrBI |
| PB2 | 1007 | EcoRV | BstZ171 | BglII |

7.1.3.4 SNP Database and SNP Analysis Tools

The Single Nucleotide Polymorphism database (dbSNP) is a public-domain archive for a broad collection of simple genetic polymorphisms (www.ncbi.nlm.nih.gov/projects/SNP/).

A comprehensive web-based application, SNP Cutter, has been created to simplify the PCR-RFLP assay design⁸ (http://bioinfo.bsd.nchicago.edu/SNP_cutter.htm). Starting from SNP sequence data preparation, SNP Cutter performs batch and automated assay design for PCR-RFLP, using pre-selected or customizable list of restriction enzymes.

7.2 Sample Preparation and Analysis on a Droplet Actuator

The invention provides a droplet actuator device and methods for integrated sample preparation and multiplexed detection of an infectious agent, such as HIV. Using digital microfluidics technology, the droplet actuator device and methods of the invention provide the ability to perform sample preparation (e.g., plasma from whole blood) and one or more molecular assays, such as multiplexed immunoassays and qRT-PCR from a single blood sample on the same droplet actuator. The droplet actuator device uses a small sample volume (e.g., about 100 to about 200 μL) and provides for rapid and accurate detection of antibodies against HIV proteins and viral RNA. The integrated method of the invention combines two independent test methods (i.e., immunoassays and qRT-PCR) and provides both screening/diagnosis and confirmatory testing using a single blood sample.

In another embodiment, the device and methods of the invention may be used to determine the stage of HIV infection (e.g., early acute, acute, chronic). Staging of HIV infection as acute or early acute at the time of diagnosis provides further confirmation value as to whether a test is a true positive.

In yet another embodiment, the device and methods of the invention may be used for both diagnostic and treatment response monitoring.

7.2.1 A Manipulation of Physiological Fluids on a Droplet Actuator

Physiological fluids (e.g., blood sample droplets) typically contain proteins. Proteins have a tendency to irreversibly adsorb to hydrophobic surfaces (i.e., top and/or bottom substrates of a droplet actuator) and contaminate them. In addition, protein adsorption may alter the hydrophobicity of the top and/or bottom substrates. Because efficient electrowetting is dependent on hydrophobic surfaces, droplet operations such as transport, mixing, and/or splinting may be adversely affected by adsorbed proteins. Different methods may be used to limit contact between a liquid droplet that contains proteins and the hydrophobic surfaces.

In one embodiment, contact between a liquid droplet that contains proteins and the hydrophobic surfaces of a droplet actuator may be controlled by use of an appropriate filler fluid. The filler fluid may be selected for compatible operation with a wide range of physiological fluids, such as whole blood, serum, plasma, and assay reagents (e.g., magnetic beads, secondary antibodies, enzymes, blocking proteins).

In another embodiment, contact between a liquid droplet that contains proteins and the hydrophobic surfaces of a droplet actuator may be controlled by the addition of certain surfactants to the oil phase (filler fluid) and/or the aqueous phase (e.g., sample droplets, reagent droplets).

7.2.2 Integration of Sample Preparation and Analysis

Two different methods may be used to separate plasma from whole blood on digital microfluidic cartridges (e.g., droplet actuators). In one embodiment, magnetically responsive immunocapture beads may be used to remove one or more populations of blood cells from a whole blood sample. In another embodiment, lateral flow plasma separation filters or vertical flow filters may be used to separate plasma from whole blood. Subsequent to preparation of a sample, molecular diagnostic assays, such as immunoassays and/or qRT-PCR, may be performed simultaneously on the same droplet actuator cartridge. For example, a first operation on-cartridge may be to prepare plasma from a whole blood sample. After preparation of plasma, droplets (e.g., 6 droplets) may be dispensed on-chip for multiple immunoassays (e.g., 6 different immunoassays). The remaining plasma sample may be dispensed for purification of RNA (e.g., viral RNA) followed by qRT-PCR.

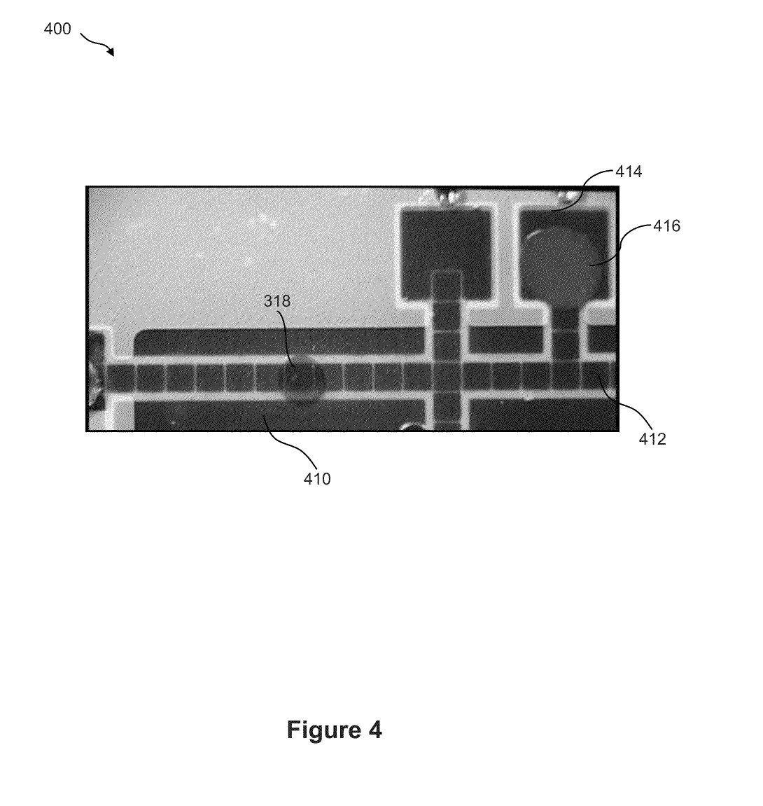

FIG. 4 illustrates a top view of an example of a portion of an electrode arrangement 400 of a droplet actuator (not shown) and shows a process of dispensing and transporting a droplet of whole blood. Electrode arrangement 400 may be disposed on a bottom substrate 410. Electrode arrangement 400 includes a path or array of droplet operations electrodes 412 and a sample reservoir 414 arranged on bottom substrate 410. Sample reservoir 414 may contain a quantity of sample fluid 416, such as whole blood, for dispensing and preparation on electrode arrangement 400. For example, a droplet 418 of whole blood may be dispensed from sample reservoir 414 using droplet operations and transported via electrowetting along droplet operations electrodes 410 for further processing and analysis.

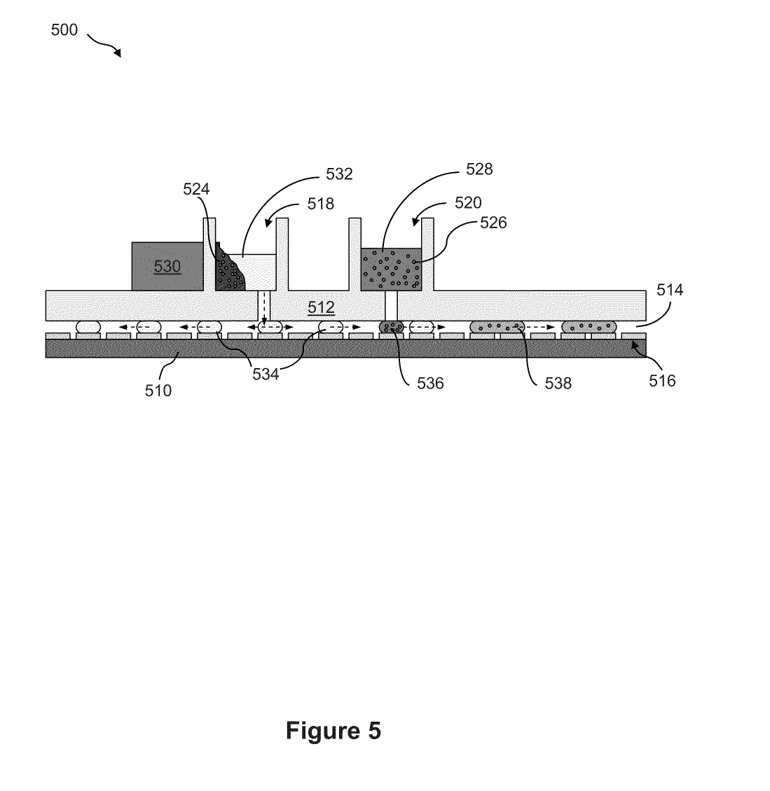

FIG. 5 illustrates a side view of an example of a portion of a droplet actuator 500 and shows a process of sample preparation from whole blood. The method of the invention of FIG. 5 is an example of integrating on-cartridge processing of whole blood samples with one or more molecular assays to provide low complexity sample-to-result analysis of a biological sample. In one embodiment, the method of the invention may be used for analysis of HIV RNA by qRT-PCR (i.e., determination of viral load) and immunoassay quantitation of antibodies directed against HIV proteins. The method of the invention uses three magnetic bead separation steps to generate purified HIV RNA free of any PCR inhibitors and remove any material that may interfere with or reduce the signal output for immunoassays.

Droplet actuator 500 may include a bottom substrate 510 and a top substrate 512 that are separated by a gap 514. Bottom substrate 510 may include a path or array of droplet operations electrodes 516 (e.g., electrowetting electrodes). Droplet operations are conducted atop droplet operations electrodes 516 on a droplet operations surface. A sample reservoir 518 and a reagent reservoir 520 may be provided in top substrate 512. Fluid paths are provided from sample reservoir 518 and reagent reservoir 520 into gap 514 such that liquid flowed from sample reservoir 518 and reagent reservoir 520 may interact with droplet operations electrodes 516. Sample reservoir 518 and reagent reservoir 520 may be of sufficient size to contain, for example, about 10-500 μL of fluid. Sample reservoir 518 may contain a quantity of magnetically responsive capture beads 524. Capture beads 524 may, for example, be DYNABEADS® beads that are coated with anti-human blood cell IgG, (available from Abcam Inc., Cambridge Cambridgeshire, UK). In another example, capture beads 524 may be Protein A coated DYNABEADS® beads (2.8 μM) that are coated with anti-human red blood cell (RBC) IgG. In yet another example, appropriate magnetic immunocapture beads may be used to remove one or more different sub-populations of cells and/or all cells from whole blood. Reagent reservoir 520 may contain a quantity of another type of capture beads 526 that are nucleic acid capture beads suspended in a lysis buffer solution 528.

A magnet 530 may be associated with droplet actuator 500. Magnet 530 may, for example, be a permanent magnet or an electromagnet. In one example, magnet 530 is a permanent magnet whose position is adjustable. For example, magnet 530 may be moved into and out of proximity with sample reservoir 518. When in close proximity to sample reservoir 518, magnet 530 may, for example, be used to attract and/or immobilize magnetically responsive capture beads 524 to one side of sample reservoir 518. In operation, magnet 530 may be used to assist in a process of removing blood cells from a whole blood sample.

An example of a process of integrating sample preparation (e.g., from whole blood) with molecular diagnostic assays (e.g., qRT-PCR, immunoassays) may include, but is not limited to, the following steps.

In a first step, a quantity of whole blood sample 532 is loaded into sample reservoir 518 that contains magnetically responsive capture beads 524 and incubated (with mixing) for a sufficient period of time to allow for the formation of capture antibody-blood cell complexes.

In a second step, after substantially all blood cells have bound to capture beads 524, magnet 530 is moved into proximity of sample reservoir 518, such that the bead-blood cell complexes are pulled to one side of sample reservoir 518 effectively separating blood cells from plasma.

In a third step, in order to integrate sample preparation with sample analysis on the same droplet actuator cartridge, whole blood sample 532, now devoid of blood cells (i.e., plasma), may be dispensed in, for example, 1 μL plasma droplets 534. For example, 6 substantially identical 1 μL plasma droplets 534 may be dispensed from sample reservoir 518 onto droplet operations electrodes 516 for immunoassays and about 100 lμl plasma droplets 534 for qRT-PCR.

Six immunoassays may be performed simultaneously on separate lanes not shown) of droplet actuator 500. For example, a plasma droplet (e.g., a 1 μL plasma droplet) may be combined with one droplet of immune-capture beads and secondary reporter antibody. After an incubation period, the bead-immune complex may be anchored in place by a magnetic field and then washed extensively to remove all materials that may interfere with signal formation or detection. The fluorescent signal from the immune complex is then measured.

Nucleic acid, such as HIV RNA, may be purified from plasma droplets 534 using the nucleic acid capture beads 526 that are suspended in lysis buffer solution 528 in reagent reservoir 520. A lysis/bead reagent droplet 536 is dispensed from reagent reservoir 520. Plasma droplets 534 are then mixed with lysis/bead reagent droplet 536 in a ratio of four plasma droplets 534 to one lysis/bead reagent droplet 536 to form a reaction droplet 538. Reaction droplet 538 is then incubated on-cartridge. Capture heads 526 with the bound HIV RNA are held in place with a magnetic field (not shown) and then washed extensively to remove all unbound material. Purified HIV RNA is then eluted from the beads with 10 mM Tris HCl, pH 8.0. This purification method produces a sample which is free of all material that may inhibit or interfere with qRT-PCR. The purified HIV RNA is now merged on-cartridge with reagents qRT-PCR and reverse transcription initiated. Amplified DNA is measured on-cartridge by fluorescence using a method, such as TAQMAN® probes (available from Life Technologies Corporation, Carlsbad, Calif.), or generic DNA intercalators, such as EVAGREEN® dye (available from Biotium, Hayward, Calif.), to determine the viral load.

In another embodiment, separate sample wells (e.g., two sample wells) may be used for preparing and dispensing plasma for molecular assays. For example, one well may be used to dispense plasma for PCR assays and a second well may be used to dispense plasma for immunoassays. In one example, whole blood samples may be loaded into each well separately. In another example, a single sample of whole blood may be directed into two (or more) separate compartments during loading.

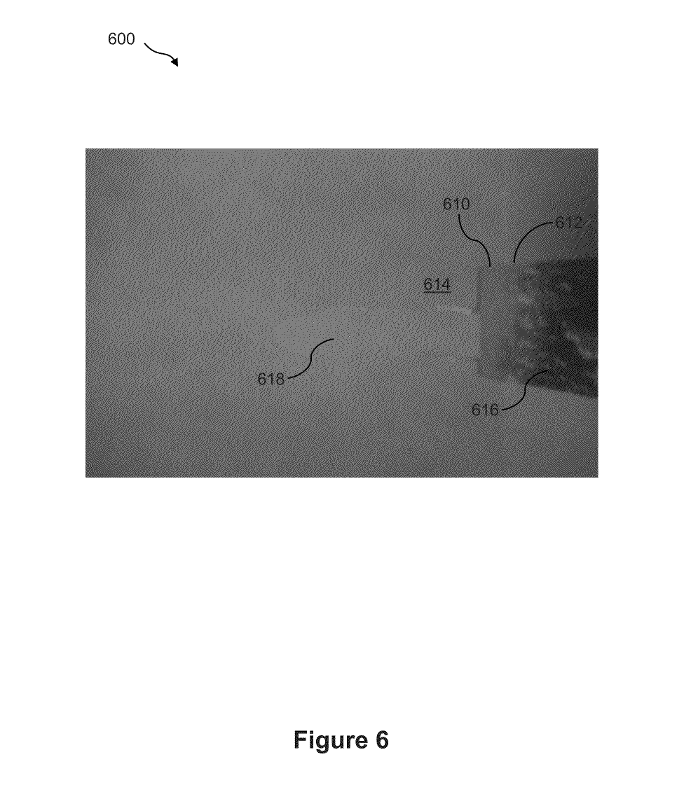

In yet another embodiment, a lateral flow plasma separation filter (e.g., a Whatman filter) and/or a vertical flow filter (e.g., a Pall filter) may be used to separate plasma from whole blood. FIG. 6 illustrates a top view of an example of a portion of an electrode arrangement 600 of a droplet actuator (not shown) and shows a process of extracting and loading plasma onto a droplet actuator using a Whatman separation filter. A filter 610 is positioned between a sample well 612 and an interior sample reservoir 614 of electrode arrangement 600. A whole blood sample 616 is loaded into the sample well. The volume of whole blood sample is sufficiently larger than the capacity of the filter paper which causes a plasma fluid 618 to flow by capillary action into interior sample reservoir 614. In one example, 1 μL of plasma fluid 618 may be extracted from 15 μL of whole blood sample 616 in about 60 seconds. The speed of extraction is dependent on the volume of excess whole blood and the capillarity of the extraction device.

In yet another embodiment, a plasma sample and a lysis solution that contains a quantity of nucleic capture beads may be combined in a single sample well and subsequently concentrated and processed on a droplet actuator.

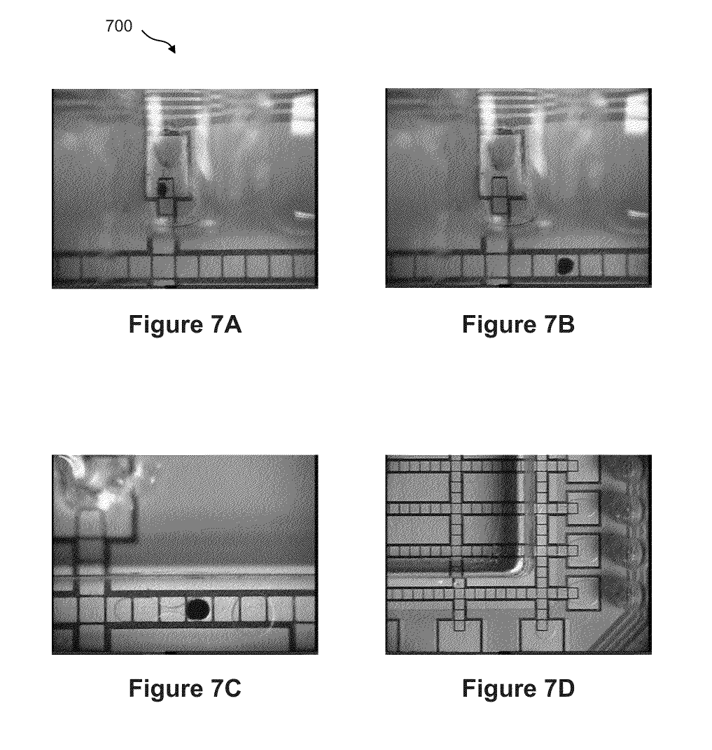

FIGS. 7A through 7D illustrate top views of an example of a portion of an electrode arrangement 700 of a droplet actuator (not shown) and show a process of detecting methicillin-resistant Staphylococcus aureus (MRSA) using digital microfluidics PCR MRSA DNA is added to several milliliters of a cell lysis solution that contains a quantity of charge-switch DNA-capture beads. The beads are then concentrated off-chip and transferred in 15 μL of solution to the sample well of a droplet actuator. Referring to FIG. 7A, a permanent magnet (not shown) in proximity of the sample well is used to collect the DNA capture beads at the bottom of the sample well. Referring to FIG. 7B, a single 300 nL droplet that contains virtually all the beads from the original sample are then dispensed from the reservoir, effectively concentrating the beads by a factor of about 50 or more. Referring to FIG. 7C, the bead-containing droplet are transported away from the sample reservoir and washed with 8 droplets of TE buffer (pH 7.0) and then eluted with 12 droplets of TE buffer (pH 8.5) into a reservoir. Referring to FIG. 7D, droplets of purified DNA are then dispensed and mixed in a 1:1 ratio with a commercial real-time PCR mix. A real-time PCR reaction is then performed and the target DNA may be detected. Because digital microfluidics is used, sensitivity of a PCR reaction is preserved while operating on sub-microliter volumes.

7.2.2.1 Sample Concentration and Dilution

The flexibility and programmability (e.g., independent control of droplet operations electrodes) of a droplet actuator provides for optimization of reaction conditions specific for a molecular assay. For example, assay parameters, such as incubation time, temperature, and number of washes, may be readily optimized for each assay type. In addition, advanced operations, such as auto-dilution and/or sample concentration, may be readily implemented on a droplet actuator. For example, when an assay reports a value above the linear range of the assay, the sample may be automatically mixed with a diluent and reanalyzed. Similarly, when the assay reports a value below the linear range of the assay, multiple sample droplets may be combined using droplet operations with a capture bead-containing droplet to increase capture of an analyte and thereby improve the limit of detection.

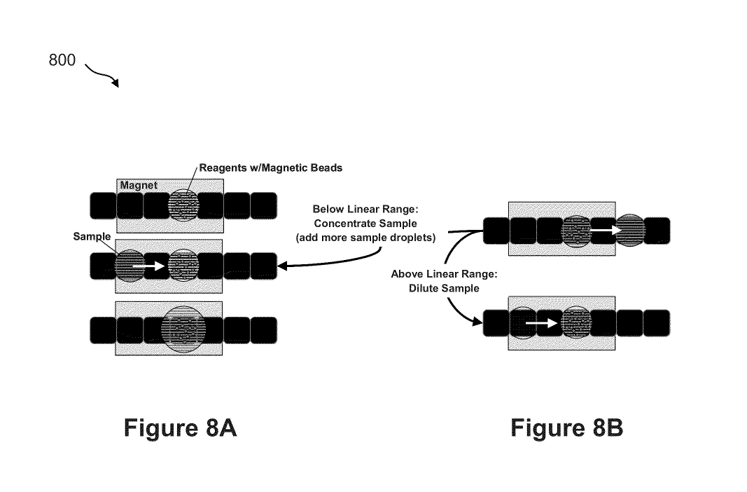

FIGS. 8A and 8B illustrate top views of an example of a portion of an electrode arrangement 800 of a droplet actuator (not shown) and show a process of concentrating or auto-diluting a sample in an immunoassay. Referring to FIG. 8A, multiple sample droplets may be dispensed and successively incubated with a single reaction droplet that contains magnetically responsive capture beads, blocking proteins, and secondary reporter antibody to concentrate the analyte within a single droplet. Because the signal output in an immunoassay is proportional to the amount of analyte captured, the sensitivity of the assay may be increased by increasing the number of sample droplets that are incubated with the magnetic beads. Referring to FIG. 8B, when a sample is above the linear range of the assay, one or more diluent droplets may be combined with the sample droplet to yield a diluted sample ready for reanalysis.

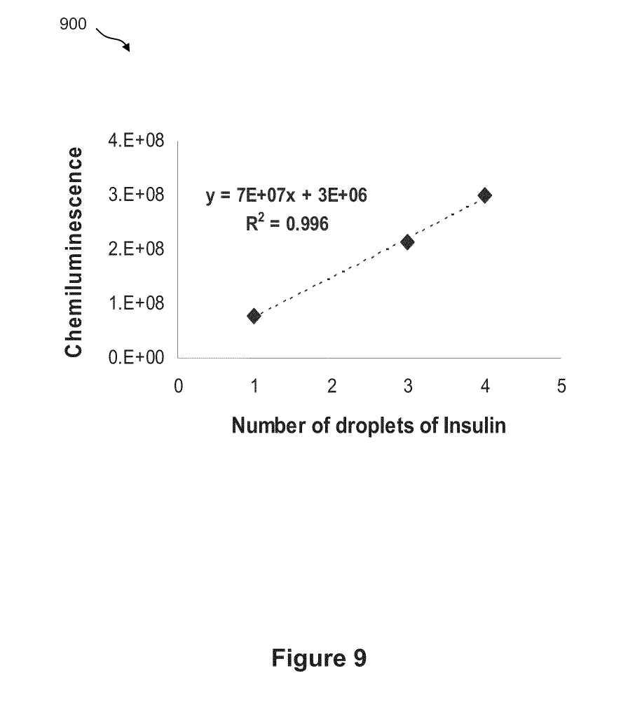

FIG. 9 shows an example of a plot 900 of chemiluminescence data for signal improvement by sample concentration in an immunoassay. Plot 900 shows an example of the results of performing an insulin immunoassay in which one, three, or four 200 nL droplets of a 7 pmol/L insulin standard solution are combined with a single magnetic bead droplet in different experiments. An increase in the number of sample droplets combined with the magnetic head droplet results in a proportional increase in signal output. By repeating the same experiment using a control solution (i.e., no insulin standard), it has been demonstrated that increasing the number of droplets that are combined with the magnetic bead droplet results in a minimal increase in background signal (data not shown). These results demonstrate that the limit of detection or the dynamic range of the assay may be improved at the lower end of sample detection by concentrating the sample.

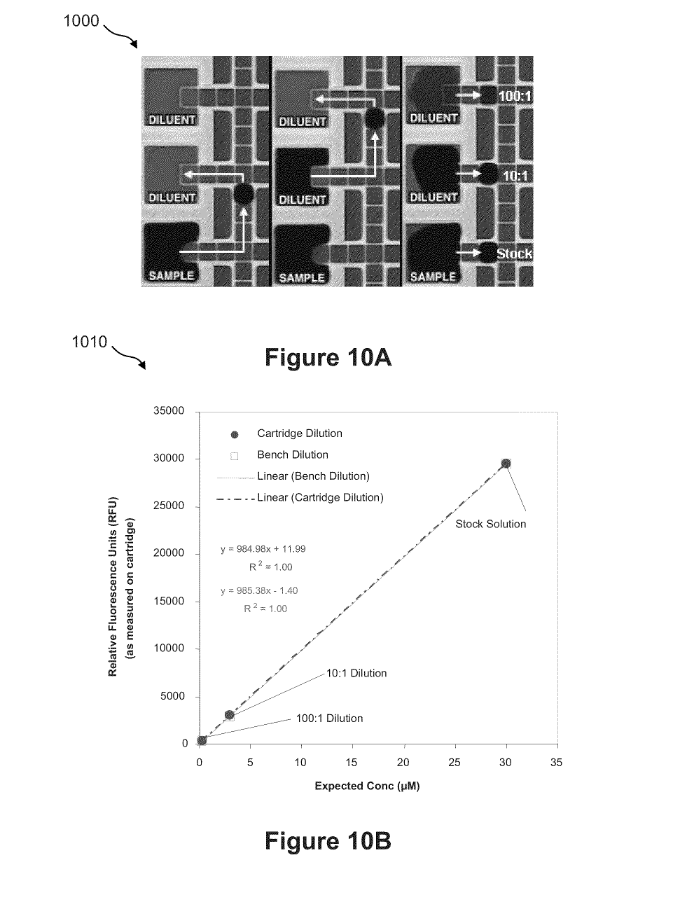

FIG. 10A illustrates a top view of an example of a portion of an electrode arrangement 1000 of a droplet actuator (not shown) and shows a process of performing an on-chip dilution protocol.

FIG. 10B illustrates an example of a plot 1010 of the results of the on-chip dilution protocol. Referring to FIG. 10A, a solution of 30 μM methylumbelliferone (fluorescence excitation 360 nM and emission 440 nM) in 0.1 M Na₂CO₃ may be diluted with 0.1 M Na₂CO₃ (diluent) by mixing 10 droplets of diluent with one droplet of solution to yield a 10:1 dilution. Subsequently, one droplet of the 10:1 dilution may be mixed with 10 droplets of diluent to form a 100:1 dilution. Mixing may be performed in an on-chip reservoir to accommodate the required 10× volume. Droplets of the stock solution, the 10:1 dilution and the 100:1 dilution are then transported to a fluorimeter and the fluorescent signal measured. Referring to plot 1010 of FIG. 10B, the results of this dilution protocol show that the methylumbelliferone concentration of the on-chip dilutions substantially matches those of comparable solutions that have been mixed manually on the bench and demonstrate the accuracy of the on-chip dilution protocol.

7.2.3 Multiplexed Analysis of HIV

Multiplexed analysis for HIV detection includes multiple immunoassays to determine the antibody response against a group of HIV proteins (e.g., gp41, gp120, Gag, RT, Tat, and Nef) and qRT-PCR to determine HIV viral load. All of the assays may be performed on a single 100-200 μL sample of blood. The sensitivity requirement for very early detection of antibodies directed against HIV proteins is 10 ng/mL blood; although concentrations of these antibodies may reach into the 10's ug/mL range. The limit of detection requirement for HIV is 200 copies viral RNA/mL blood. A relatively small fraction of the entire plasma (e.g., about 6 μL) is required for performing the multiplexed immunoassays for antibodies to HIV proteins to achieve the required sensitivity of detection. The remainder of the plasma sample may be used for HIV viral RNA detection to maximize the sensitivity of the qRT-PCR assay. On-cartridge detection for both immunoassays and qRT-PCR may, for example, be a fluorescence-based detection method.

7.2.3.1 Immunoassays for Antibodies to HIV Proteins

The immunoassay format for antibodies against six HIV proteins, gp41, gp120, Gag, RT, Tat and Nef, are substantially the same. Each assay may be validated on-bench prior to implementation on-cartridge. Recombinant HIV proteins and/or synthetic peptides representative of each of the six target antigens may be conjugated to magnetic capture beads (e.g., 2.8 μM Dynal magnetic capture beads). The assay detection method may be fluorescence-based. The reporter antibody may, for example, be goat anti-human IgG or IgM conjugated to fluorescein, phycoerythrin, or alkaline phosphatase. The limit of detection for each of the fluorescent labels may be experimentally determined. Commercially available antibodies specific for each of the six HIV target proteins may be used as test standards and used to generate standard curves.

For each immunoassay, a plasma droplet (e.g., 1 μL droplet) may be combined using droplet operations with a droplet that contains a quantity of specific capture beads. After an incubation time of about 1 to 2 minutes, one droplet of reporter antibody may be merged with the bead/sample droplet. After an additional incubation period of about 1 to 2 minutes, the beads may be washed to remove unbound material and then transported to a detector to measure the fluorescent signal. For each immunoassay, the incubation times, concentration of reporter antibody, number of washes and composition of blocking agents may be optimized to achieve minimal non-specific binding and maximum sensitivity.

7.2.3.2 qRT-PCR to Determine Viral Load

HIV testing (i.e., determination of viral load) may be performed on purified HIV RN using GIRT-PCR. Typically, the limit of detection requirement for HIV is about 200 copies/mL blood which is equivalent to 20 and 40 copies, respectively, per 100 μL and 200 μL of blood. After samples for the immunoassays have been dispensed, the entire remaining plasma sample, about from a 100 μL blood sample, may be dispensed for qRT-PCR.

To determine viral load, HIV RNA is purified on-cartridge using, for example, DYNABEADS® silane viral NA (available from Dynal Bead Based Separations (invitrogen Group), Carlsbad, Calif.). DYNABEADS® Silane viral NA beads are optimized for the purification of viral nucleic acids from human serum or plasma samples with very low numbers of viral infectious units/mL. The magnetic DYNABEADS® Silane and lysis/binding buffer may be stored on cartridge in a reagent reservoir (refer to FIG. 5). The remaining volume of a plasma sample may be dispensed onto the cartridge from the sample reservoir in 1 μL droplet volumes. A lysis/bead reagent droplet may be dispensed from the reagent reservoir. The plasma droplets may be mixed with a lysis/bead droplet in a ratio of four plasma droplets to one lysis/bead droplet and incubated on-cartridge. Capture beads with the bound HIV RNA may be held in place with a magnetic field and then washed extensively to remove all unbound material. Purified HIV RNA may then be eluted from the beads with 10 mM Tris HCl, pH 8.0. This purification method produces a sample which is free of all material which may inhibit or interfere with qRT-PCR. The purified HIV RNA may then be merged on-cartridge with reagents for qRT-PCR and reverse transcription initiated. Amplified DNA may be measured on-cartridge by fluorescence using a method, such as TAQMAN® probes, TAQMAN® or generic DNA intercalators, such as EVAGREEN® dye, to determine the viral load.

On-cartridge qRT-PCR may be further optimized for quantitation of HIV load. For example, a specific HIV gene target may be used for amplification. Additional primer sequences for optimal reverse transcription and PCR may be designed. qPCR detection methods, such as TAQMAN® probes, TAQMAN® or a generic dye, such as EVAGREEN® dye, may be evaluated for optimal detection. Amplification of an internal standard may be used to normalize samples in a case of sample interferents.

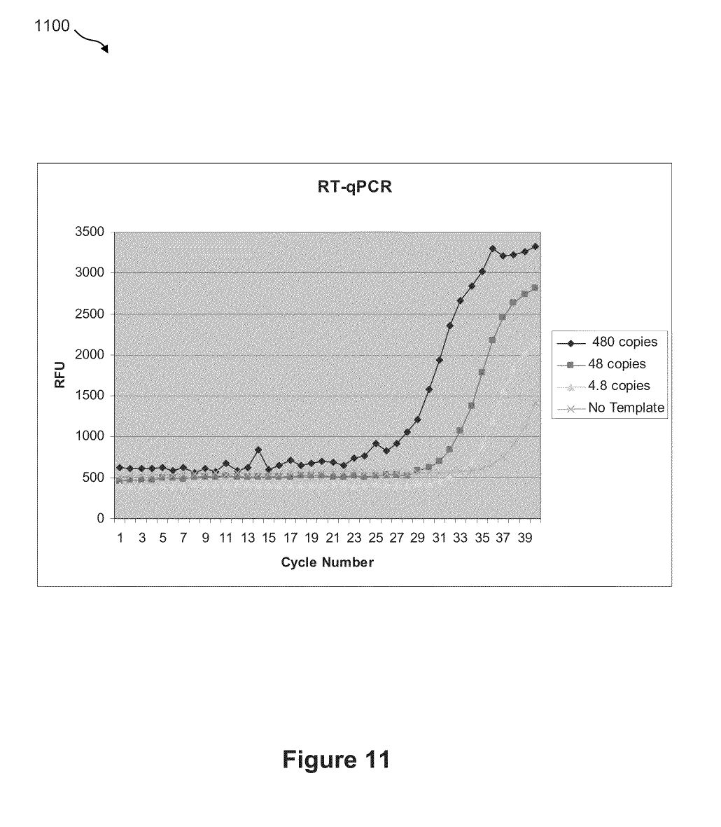

FIG. 11 shows an example of a plot 1100 of qRT-PCR data for detection of a RNA transcript by use of digital microfluidics. A dilution series of a synthetic RNA transcript, Xeno™ RNA Control (available from Life Technologies Corporation. Carlsbad, Calif.), was used in a reverse transcription reaction to synthesize DNA for qPCR. The amplified DNA was measured after each amplification cycle by fluorescence measurement of EvaGreen. The number of Xeno™ RNA molecules in each droplet was 480, 48, 4.8 copies or zero for the no template control sample. The results shown in plot 1100 demonstrate a conversion of the Xeno™ RNA to cDNA and amplification of the cDNA in a concentration-dependent manner. The amplification curves shown in plot 1100 have calculated C_{T} values of 27, 31 and 33 for the RNA samples containing 480, 48 and 4.8 copies, respectively. Although the no template control sample generated a late occurring signal in this experiment, the PCR droplet containing only 4.8 copies of the RNA was easily distinguishable from the no template control. This experiment demonstrates that if the capture and purification of HIV RNA from plasma is efficient, there is sufficient sensitivity on-cartridge in the qRT-PCR step to detect HIV at a LOD of 200 copies/mL of blood.

7.2.4 Blood Sample Collection

Several approaches may be used for sample collection including venipuncture and finger stick. Typically, these methods include the use of an anticoagulant, such as anticoagulant citrate dextrose (ACD) or ethylene diamine tetra-acetic acid (EDTA)., in the collection tube. For integrated sample preparation and analysis using digital microfluidics, a small sample volume of about 200 μL is required. This sample volume may be collected via a finger stick using a commercially available finger stick collection device (e.g. BD MICROTAINER® tubes, available from Becton, Dickinson and Company, Franklin Lakes, N.J.). Volumes of 200 μL may be required to obtain HIV viral load copy sensitivity down to 50 copies/mL. However, for a higher cut off of 500 or 1000 copies/mL, the overall blood draw requirements may be reduced to as low as 50 μL of blood. Current guidelines for ART use and monitoring sets the lower limit of detection of 1000 copies/mL as the global standard (2008 UNAIDS/WHO consensus meeting).

7.3 Additional Molecular Techniques for Digital Microfluidic

The invention provides droplet actuator devices and techniques for PCR amplification and detection of specific nucleic acid sequences using digital microfluidics. The methods of the invention generally involve combining the necessary reactants to form a PCR-ready droplet and thermal cycling the droplet at temperatures sufficient to result in amplification of a target nucleic acid. A droplet including the amplified target nucleic acid may then be transported into a subsequent process, such as a detection process. The droplet actuator device uses a small sample volume and provides for rapid and accurate amplification and detection of target nucleic acid sequences. In various embodiments, the invention also provides for droplet actuator-based sample preparation and target nucleic acid analysis. Combining amplification and detection steps on a droplet actuator provides for rapid and flexible investigation of DNA sequences.

In one embodiment, the invention provides methods for performing hot-start PCR on a droplet actuator. Hot-start PCR is typically used to reduce non-specific amplification during the initial set up stages of a PCR assay. In one example, methods of the invention include physical separation of the amplification enzyme (e.g., DNA polymerase) from the reaction mixture until a sufficient temperature (e.g., DNA melting temperature) is achieved. In another example, DNA polymerase may be maintained in an inactive state until a sufficient temperature is achieved.

In another embodiment, the method of the invention combines PCR amplification with various sequence specific detection technologies for amplified DNA. In one example, one or more PCR primers may be labeled. The label may be selected to provide a signal, such as a fluorescent signal, and/or selected to facilitate isolation/immobilization of the amplified product. In another example, labeled oligonucleotide probes, e.g., fluorescently labeled probes may be used for hybridization-based detection. Fluorescence-based detection techniques may be used for end-point or real-time analysis of DNA amplification. For end-point analysis, the accumulation of a signal, e.g., a fluorescence signal, is measured after the amplification of the target sequence is complete. For real-time analysis, the signal is measured while the amplification reaction is in progress. In another example, pyrosequencing may be used to investigate specific sequences.

In another embodiment, the method of the invention combines PCR amplification with pyrosequencing to investigate specific sequences. In one example, protocols for DNA amplification (PCR) and DNA sequencing of a target sequence may be performed on a single droplet actuator. In another example, protocols for isolation of nucleic acid (e.g., genomic DNA) from a biological sample, DNA amplification and DNA sequencing may be performed on a single droplet actuator. Integration of sample preparation, DNA amplification and sequencing on a single digital microfluidic devices provides for rapid and reliable sample-to-result detection of target nucleic acid sequences.

In certain embodiments, the devices and methods of the invention may be used for amplification and detection of specific nucleotide sequences, such as pathogen nucleic acids (e.g., bacteria, virus, fungus or parasite). In other embodiments, the devices and methods of the invention may be used to distinguish SNP alleles. SNPs are the most common source of genetic variation in humans. SNPs are DNA sequence variations that occur when a single nucleotide (adenine (A), thymine (T), cytosine (C), or guanine (G)) in the genome sequence is altered. SNPs are the most common markers for both genes associated with disease (medical diagnostics) and drug response associations (pharmacogenetics). Effective implementation of diagnostic genotyping and/or pharmacogenetics in a clinical setting (point-of-care) requires testing of a patient at presentation to facilitate diagnosis and/or treatment decisions.

7.3.1 Hot-Start PCR

PCR applications typically use a heat-stable DNA polymerase, such as Taq polymerase, and thermal cycling (e.g. repeated cycles of denaturation and annealing/extension) to amplify one or more target genes of interest. One significant problem with PCR is the potential for the generation of nonspecific amplification products. These nonspecific amplification products are often a result of inappropriate oligonucleotide priming and subsequent primer extension by DNA polymerase prior to the actual thermocycling procedure. Nonspecific amplification may be minimized by limiting polymerase activity prior to PCR cycling. For example, nonspecific amplification may be minimized by incorporation of various hot-start techniques into the PCR protocol prior to the actual thermocycling procedure.

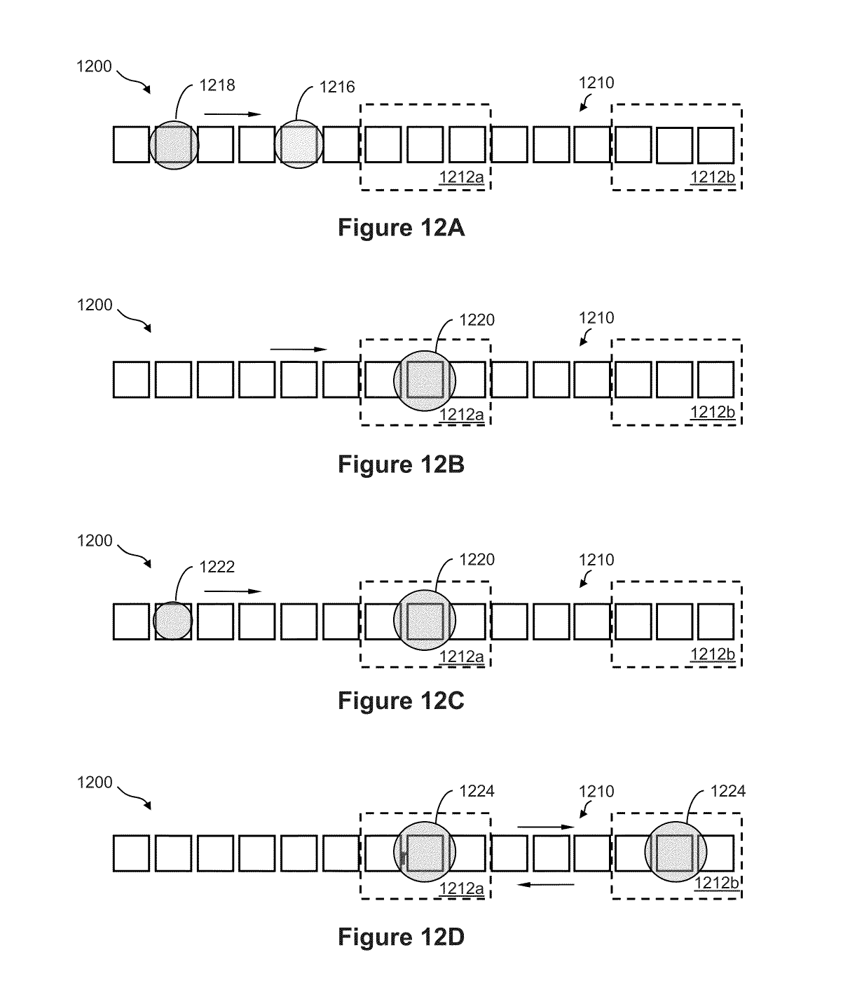

FIGS. 12A through 12D illustrate top views of an example of a portion of an electrode arrangement 1200 of a droplet actuator (not shown) and a process of performing a hot-start protocol. The method of the invention of FIGS. 12A through 12D is an example of a hot-start protocol in which a DNA sample and PCR reagents (e.g., primers, deoxynucleotides, buffers) are combined and incubated at an elevated temperature (e.g., about 95-100° C.) prior to addition of DNA polymerase.

Electrode arrangement 1200 may include an arrangement of droplet operations electrodes 1210 (e.g., electrowetting electrodes) that is configured for PCR analysis. Droplet operations are conducted atop droplet operations electrodes 1210 on a droplet operations surface. Two temperature control zones 1212, such as temperature control zone 1212a and 1212b may be associated with electrode arrangement 1200. Thermal control elements (not shown) control the temperature of filler fluid (not shown) in vicinity of temperature control zones 1212a and 12121. For example, temperature control zone 1212a may be heated to about 95° C. (melting temperature), which is a temperature sufficient for denaturation of DNA template and primers. Temperature control zone 1212b may, for example, be heated to about 60 to 72° C., which is a temperature sufficient for annealing of primer to the single-stranded DNA template and primer extension by DNA polymerase. While two temperature control zones 1212 are shown, any number of temperature control zones 1212 may be associated with electrode arrangement 1200.

A sample droplet 1216 may be transported using droplet operations along; droplet operations electrodes 1210. In one embodiment, sample droplet 1216 may contain purified DNA to be evaluated by PCR analysis. An example of a process of performing hot-start PCR on a droplet actuator may include, but is not limited to, the following steps.

In one step, FIG. 12A shows sample droplet 1216 that is positioned at a certain droplet operations electrode 1210. A reagent droplet 1218 is positioned in proximity of sample droplet 1216. In one example, reagent droplet 1218 includes PCR reagents, such as deoxynucleotides, primer pairs, magnesium salt and buffers.

In another step, FIG. 12B shows an incubation process in which reagent droplet 1218 is merged using droplet operations with sample droplet 1216 to form a reaction droplet 1220. Reaction droplet 1220 is transported using droplet operations to a certain droplet operations electrode 1210 within temperature control zone 1212a. Reaction droplet 1220 is incubated in temperature control zone 1212a for a period of time that is sufficient to dissociate the target DNA to free single stranded template and denature any primer-dimer pairs.

In another step, FIG. 12C shows a second reagent droplet 1222 that includes a quantity of an amplification enzyme, such as DNA polymerase, positioned at a certain droplet operations electrode 1210 in proximity to temperature control zone 1212a and reaction droplet 1220 therein.

In another step, FIG. 12D shows another incubation process in which reagent droplet 22 is merged using droplet operations with reaction droplet 1220 within temperature control zone 1212a to form a complete reaction droplet 1224. Reaction droplet 1224 now includes all the components required for PCR amplification of target DNA. Reaction droplet 1224 is transported using droplet operations to temperature control zone 1212b. Reaction droplet 1224 is incubated within temperature control zone 1212b for a period of time that is sufficient for annealing of primers to the target single stranded DNA template and extension of the annealed primers by DNA polymerase. Reaction droplet 1224 is then transported using droplet operations back to temperature control zone 1212a to initiate another round of DNA amplification. Reaction droplet 1224 may be repeatedly transported between temperature control zones 1212a and 1212b any number of times sufficient for a desired level of DNA amplification.

In another embodiment, at the step shown in FIG. 12C, reagent droplet 1222 (containing DNA polymerase) may be positioned at a certain droplet operations electrode 1210 within temperature control zone 1212b. Then at the step shown in FIG. 12D, reaction droplet 1220 may be transported from temperature control zone 1212a and merged with reagent droplet 1222 within temperature control zone 1212b to form merged reaction droplet 1224.

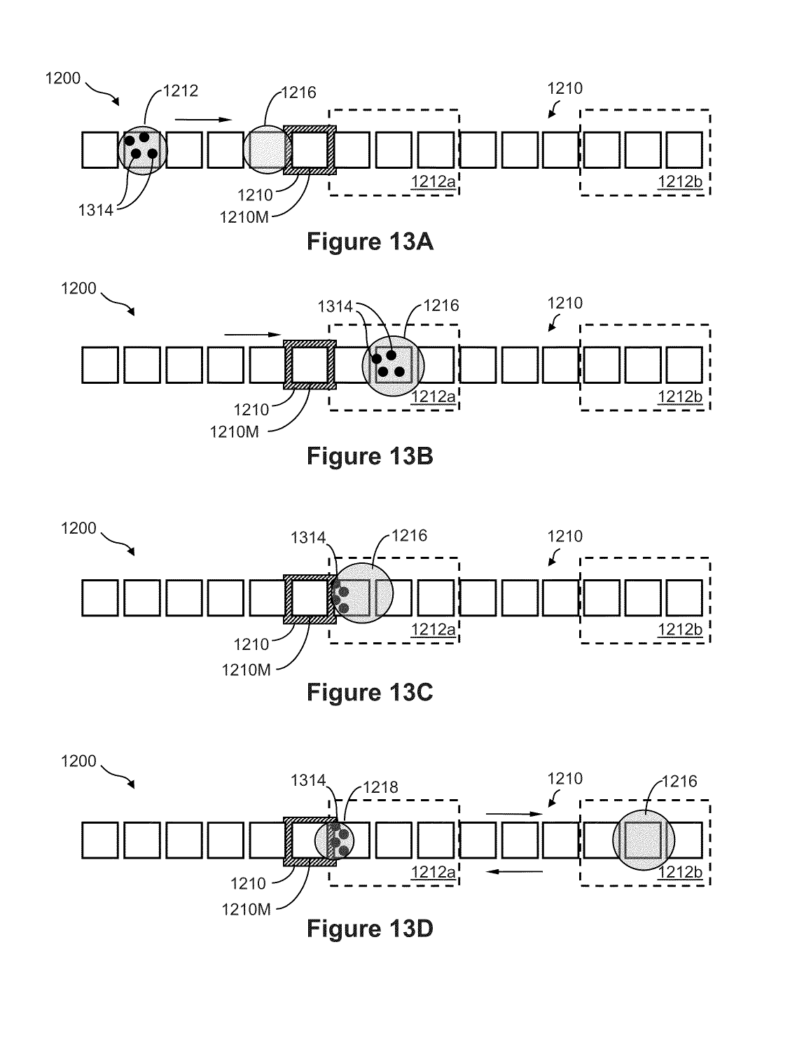

FIGS. 13A through 13D again illustrate top views of the electrode arrangement 1200 of FIGS. 12A through 12D and show a process of performing a hot-start protocol that uses DNA polymerase immobilized on magnetically responsive beads. In this embodiment, a magnet 1310 is provided in proximity to temperature control zone 1212a for retaining a quantity of magnetically responsive beads 1314. In particular, magnet 1310 is arranged such that a certain droplet operations electrode 1210 (e.g., droplet operations electrode 1210M) is within the magnetic field thereof. Magnet 1310 may, for example be a permanent magnet or an electromagnet. In this embodiment, instead of providing DNA polymerase as a separate reagent droplet, the DNA polymerase is immobilized on magnetically responsive beads and included in a single PCR reagent droplet. Because the DNA polymerase is immobilized on the beads, it is inactive. An example of a process of performing a hot-start protocol that uses DNA polymerase immobilized on magnetically responsive beads may include, but is not limited to, the following steps.

In one step, FIG. 13A shows sample droplet 1216 that is positioned at a certain droplet operations electrode 1210. A reagent droplet 1312 that includes a quantity of magnetically responsive beads 1314 with DNA, polymerase immobilized thereon is positioned in proximity of sample droplet 1216.

In another step, FIG. 13B shows an incubation process in which reagent droplet 1312 that includes a quantity of magnetically responsive beads 1314 with DNA polymerase immobilized thereon is merged using droplet operations with sample droplet 1216 to form a complete reaction droplet 1316. In one example, magnetically responsive beads 1314 may be coated with an antibody that specifically binds DNA polymerase at ambient temperature. Reaction droplet 1316 is transported using droplet operations to a certain droplet operations electrode 1210 within temperature control zone 1212a. Reaction droplet 1316 is incubated in temperature control zone 1212a for a period of time that is sufficient to denature target DNA and any primer-duper pairs. At the elevated temperature within temperature control zone 1212a. DNA polymerase is released from magnetically responsive beads 1314 into the reaction mixture and is active.

In other steps, FIGS. 13C and 13D show a splitting process in which magnetically responsive beads 1314 may be removed from reaction droplet 1316 after DNA polymerase is released from magnetically responsive beads 1314 by heating. Reaction droplet 1316 with magnetically responsive beads 1314 therein is transported into the magnetic field of magnet 1310 such that magnetically responsive beads 1314 are attracted to and held by the magnetic field. Reaction droplet 1316 is then transported using droplet operations (e.g., droplet operations mediated by electrowetting) away from droplet operations electrode 1210M (i.e., away from the magnetic field of magnet 1310) to temperature control zone 1212b. As reaction droplet 1316 moves away from the magnetic field, a small droplet 1318 that includes a concentration of magnetically responsive beads 1314 is formed. The small droplet 1318 that has magnetically responsive beads 1314 therein may be discarded. Reaction droplet 1316, which is now devoid of magnetically responsive beads 1314, may be cycled for PCR amplification. Because magnetically responsive beads 1314 are removed from the reaction droplet, immobilization of DNA polymerase during subsequent amplification reactions is avoided.

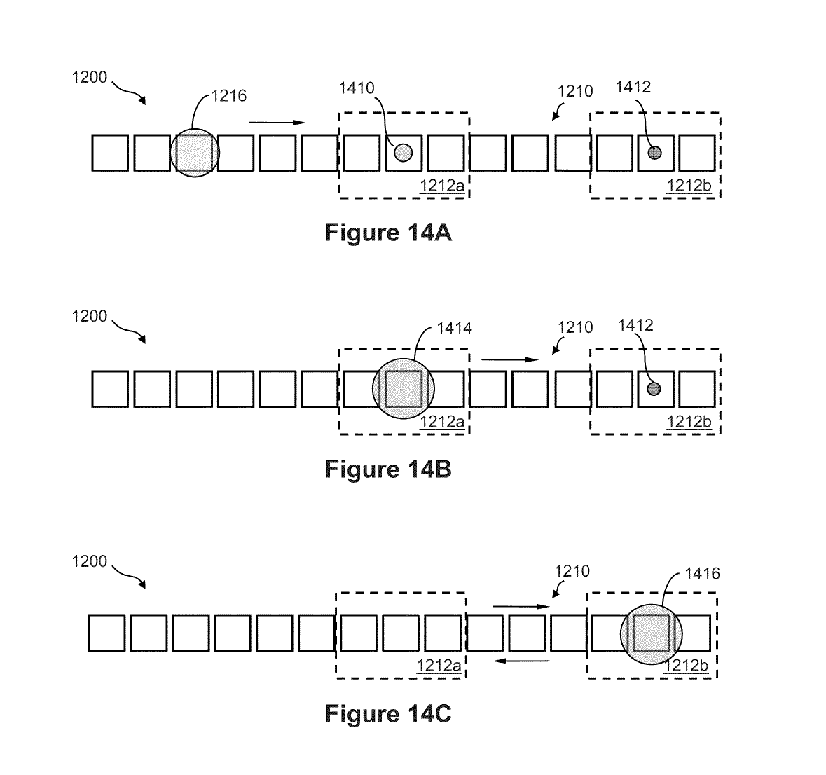

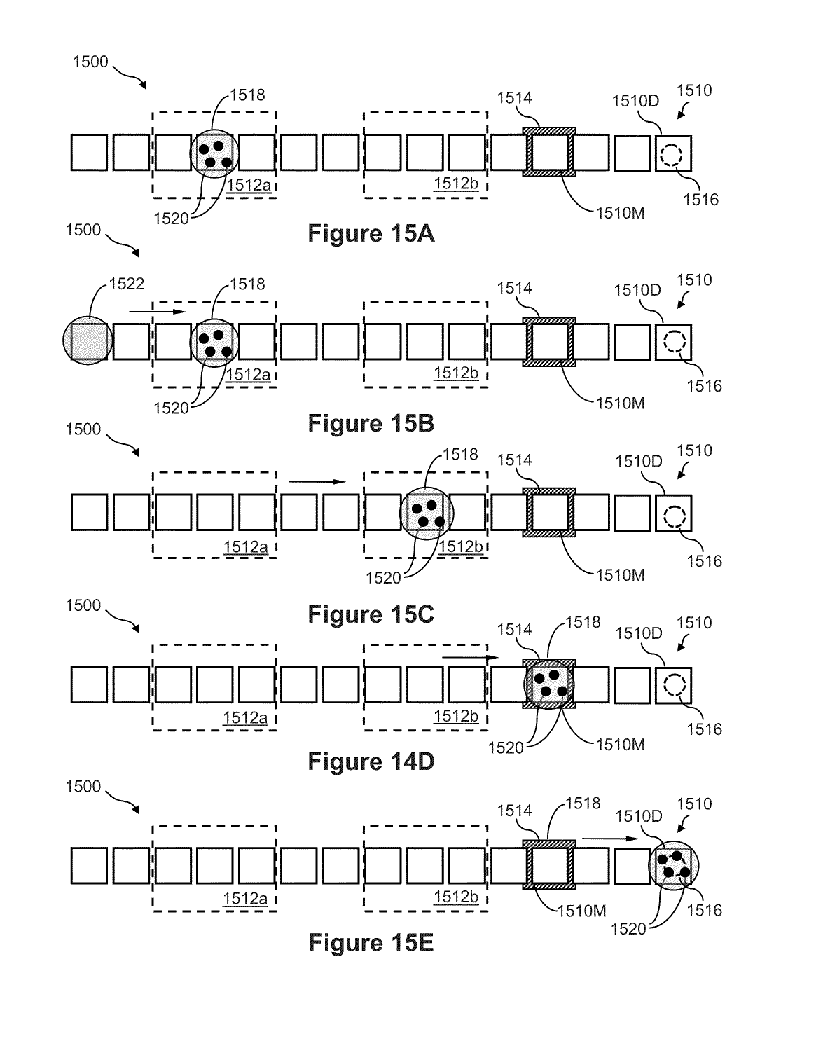

FIGS. 14A through 14C again illustrate top views of the electrode arrangement 1200 of FIGS. 12A through 12D and show a process of performing a hot-start protocol that includes reconstituting dehydrated PCR reagents. In this example, PCR reagents (e.g., primers, deoxynucleotides, buffers, DNA polymerase) are provided as dehydrated reagents deposited on certain droplet operations electrodes 1210. An example of a process of performing a hot-start protocol that includes reconstituting dehydrated PCR reagents may include, but is not limited to, the following steps.

In one step, FIG. 4A shows sample droplet 1216 that is positioned at a certain droplet operations electrode 1210. A concentrated reagent droplet 1410 is present at a certain droplet operations electrode 1210 within temperature control zone 1212a and is ready for reconstitution. Reagent droplet 1410 includes PCR reagents, such as primer pairs, deoxynucleotides, and magnesium salt that are sufficient for PCR amplification. Further, a second reagent droplet 1412 is present at a certain droplet operations electrode 1210 within temperature control zone 1212b and is ready for reconstitution. Reagent droplet 1412 includes an amplification enzyme, such as DNA polymerase. Reagent droplets 1410 and 1412 may, for example, be dried in place by manual spotting or by an automated printing device.

In other steps, FIGS. 14B and 14C show an incubation process in which sample droplet 1216 is transported using droplet operations to temperature control zone 1212a and reconstitutes reagent droplet 1410 to form a reaction droplet 1414. After an incubation period that is sufficient to dissociate the target DNA to free single stranded template and denature any primer-dimer pairs, reaction droplet 1414 is transported to temperature control zone 1212b and reconstitutes reagent droplet 1412 to yield a complete reaction droplet 1416. As target. DNA and primers anneal and the DNA polymerase in reagent droplet 1412 is rehydrated, primer extension begins. Reaction droplet 1416 may be repeatedly transported between temperature control zones 1212a and 1212b any number of times sufficient for a desired level of DNA amplification.

In another embodiment, reagent droplets 1410 and 1412 may be combined and provided as a single dehydrated reagent droplet loaded onto a certain droplet operations electrode 1210 within temperature control zone 1212a. In one example, elution buffer may be used to reconstitute the reagent droplet prior to mixing and incubation with a sample droplet. In another example, a sample droplet may be used to reconstitute the dehydrated reagent droplet.

7.3.2 Probe Hybridization-Based Detection

Probe hybridization is a general detection method which measures the hybridization of a labeled probe, e.g., a fluorescently-labeled probe, to a specific amplified DNA sequence (e.g., pathogen or SNP). Typically, thermal conditions are set up such that the probe will only hybridize to a DNA sequence that is a perfect match thereby generating a signal. Any mismatches in the target DNA will disrupt the probe hybridization resulting in no fluorescent signal. In one embodiment, the amplified nucleic acid target is anchored to a solid-support, such as a bead, to allow the excess, unbound fluorescent probe to be washed away before measuring the signal. In another embodiment, the amplified nucleic acid target is unanchored (i.e., homogeneous solution) and generation of a fluorescent signal is determined by the configuration of the probe upon hybridization to the target sequences.

FIGS. 15A through 15E illustrate top views of an example of a portion of an electrode arrangement 1500 of a droplet actuator (not shown) and show a process detecting an immobilized target sequence on a droplet actuator. The method of the invention of FIGS. 15A through 15E is an example of an end-point detection protocol in which amplified target nucleic acids (e.g., pathogen and/or SNP region) are immobilized on magnetically responsive beads prior to incubation with a fluorescently labeled probe specific for the amplified DNA sequence.

Electrode arrangement 1500 may include an arrangement of droplet operations electrodes 1510 that is configured for hybridization of nucleic acid sequences. Droplet operations are conducted atop droplet operations electrodes 1510 on a droplet operations surface. Two temperature control zones 1512, such as temperature control zone 1512a and 1512b, may be associated with electrode arrangement 1500. Thermal control elements (not shown) control the temperature of filler fluid (not shown) in the vicinity of temperature control zones 1512a and 1512b. For example, temperature control zone 1512a may be heated to about 95° C. (melting temperature), which is a temperature sufficient for denaturation of double-stranded DNA. Temperature control zone 1512b may, for example, be heated to about 55° C., which is a temperature sufficient for specific hybridization of an oligonucleotide probe to a single-stranded DNA target. In one example; temperature control zones 1512a and 1512b may be the same temperature control zones used for PCR cycling as described in reference to electrode arrangement 1200 of FIG. 12A. In another example, thermal conditions in temperature control zone 15121 may be adjusted for a given hybridization protocol such that the oligonucleotide probe will only hybridize to a DNA sequence that is a perfect match. While two temperature control zones 1512 are shown, any number of temperature control zones 1512 may be associated with electrode arrangement 1510.

A magnet 1514 is provided in proximity to temperature control zone 1512b for retaining a quantity of magnetically responsive beads. In particular, magnet 1514 is arranged such that a certain droplet operations electrode 1510 (e.g., droplet operations electrode 1510M) is within the magnetic field thereof. Magnet 1514 may, for example, be a permanent magnet or an electromagnet. A detection spot 1516 may be arranged in close proximity to droplet operations electrode 1510D.

An example of a process of probe hybridization to an anchored target sequence may include, but is not limited to, the following steps.

In one step, FIG. 15A shows a sample droplet 1518 that is positioned at a certain droplet operations electrode 1510 within temperature control zone 1512a. Sample droplet 1518 may include a quantity of magnetically responsive beads 1520 that is coated with streptavidin. In one example, the PCR amplicons may be a specific amplified DNA sequence for a pathogen or a SNP. The PCR amplicons may, for example, be formed using an amplification protocol that includes a biotinylated primer (e.g., biotinylated forward primer). The biotinylated PCR amplicon may be immobilized on magnetically responsive beads 1520 through formation of a biotin-streptavidin complex. Because sample droplet 1518 is within temperature control zone 1512a, the immobilized PCR amplicons are single-stranded (denatured).

In other steps, FIGS. 15B and 15C show an incubation process in which a reagent droplet 1522 is merged using droplet operations with sample droplet 1518 within temperature control zone 1512a. Reagent droplet 1522 includes a fluorescently oligonucleotide probe that is specific for the amplified target DNA immobilized on magnetically responsive beads 1520. The oligonucleotide probe may be labeled with a fluorophore that provides a fluorescent signal in the presence or absence of specific binding to the amplified target sequence. Merged sample droplet 1518 with magnetically responsive beads 1520 therein is transported using droplet operations to a certain droplet operations electrode 1510 within temperature control zone 1512b. Merged sample droplet 1518 is incubated in temperature control zone 1512b for a period of time that is sufficient for hybridization of the fluorescently labeled probe to single stranded target DNA sequences. Because the labeled probe provides a fluorescent signal in the presence or absence of binding to target sequences, excess unbound probe must be removed prior to detection of a specific hybridization signal.

In another step, FIG. 15D shows merged sample droplet 1518 that has magnetically responsive beads 1520 therein transported using droplet operations to droplet operations electrode 1510M (i.e., into the magnetic field of magnet 1514). A bead washing protocol, such as the bead washing protocol described in reference to FIGS. 2F and 2G may be used to remove excess unbound fluorescent probe.

In another step, FIG. 15E shows merged sample droplet 1518 that has magnetically responsive beads 1520 therein transported using droplet operations to droplet operations electrode 1510D, which is within the range of detection spot 1516. An imaging device (e.g., fluorimeter, not shown), arranged in proximity of detection spot 1516, is used to capture and quantitate the amount of fluorescence anchored on magnetically responsive beads 1520 in merged sample droplet 1518.

In another embodiment, amplification and detection protocols may include detection technologies wherein excess probe does not need to be removed prior to detection. In this example, non-biotinylated primers may be used to amplify target DNA sequences and immobilization of PCR amplicons on a solid support (i.e., magnetically responsive beads) and associated bead washing processes may be omitted.

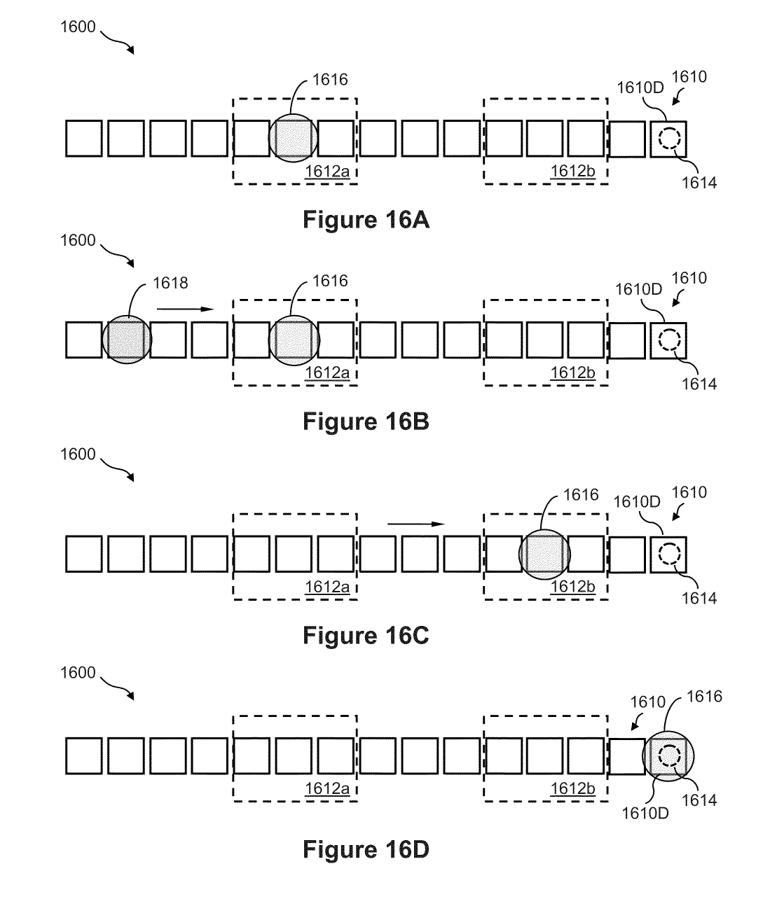

FIGS. 16A through 16D illustrate top views of an example of a portion of an electrode arrangement 1600 of a droplet actuator (not shown) and show a process of detecting an unanchored amplified target sequence on a droplet actuator. The method of the invention of FIGS. 16A through 16D is an example of a detection protocol in which unanchored nucleic acid template (e.g., pathogen and/or SNP region of interest) is amplified and hybridized to one or more fluorescently labeled oligonucleotide probes. In various embodiments, the specificity of the fluorescent signal is determined by the configuration of the probe upon hybridization to the target sequences. Because a fluorescent signal is only generated by specific binding of the probe to a target sequence, removal of excess unbound probe is not required. Amplified product may be detected and/or quantified in real-time or end-point analysis. For end-point analysis, the accumulation of a signal, e.g., a fluorescence signal, is measured after the amplification of the target sequence is complete. For real-time analysis, the signal is measured while the amplification reaction is in progress.

Electrode arrangement 1600 may include an arrangement of droplet operations electrodes 1610 that is configured for hybridization of oligonucleotide probes and target sequences. Droplet operations are conducted atop droplet operations electrodes 1610 on a droplet operations surface. Two temperature control zones 1612, such as temperature control zone 1612a and 1612b, may be associated with electrode arrangement 1600. Thermal control elements (not shown) control the temperature of filler fluid (not shown) in the vicinity of temperature control zones 1612a and 1612b. For example, temperature control zone 1612a may be heated to about 95° C. (melting temperature) which is a temperature sufficient for denaturation of double-stranded DNA. Temperature control zone 1612b may, for example, be heated to about 55° C., which is a temperature sufficient for primer annealing and extension and for specific hybridization of one or more oligonucleotide probes to a single-stranded DNA target. In one example, temperature control zones 1612a and 1612h may be the same temperature control zones used for PCR cycling as described in reference to electrode arrangement 1200 of FIG. 12A, another example, thermal conditions in temperature control zone 1612b may be adjusted for a certain amplification and hybridization protocol such that the oligonucleotide probe will only hybridize to a DNA sequence that is a perfect match. While two temperature control zones 1612 are shown, any number of temperature control zones 1612 may be associated with electrode arrangement 1610. A detection spot 1614 may be arranged in close proximity to droplet operations electrode 1610D.

An example of a general process of amplification and probe hybridization to an unanchored target sequence may include, but is not limited to, the following steps.

In one step, FIG. 16A shows a sample droplet 1616 that is positioned at a certain droplet operations electrode 1610 within temperature control zone 1612a. Sample droplet 1616 may, for example, include nucleic acid template (DNA target) for amplification. In one example, the nucleic acid template may be nucleic acid isolated from a pathogen. In another example, the nucleic acid template may include a SNP region of interest. Because sample droplet 1616 is with in temperature control zone 1612a, the nucleic acid template is single-stranded (denatured).

In other steps, FIGS. 16B and 16C show an incubation process in which a reagent droplet 1618 is merged using droplet operations with sample droplet 1616 within temperature control zone 1612a. Reagent droplet 1618 may include primers and PCR reagents (e.g., dNTPs, buffers, DNA polymerase) for target amplification. Reagent droplet 1618 may also include one or more fluorescently labeled oligonucleotide probes that are specific for the DNA target. Merged sample droplet 1616 is transported using droplet operations to a certain droplet operations electrode 1610 within temperature control zone 1612b. Merged sample droplet 1616 is incubated in temperature control zone 1612b for a period of time that is sufficient for primer annealing/extension and hybridization of the fluorescently labeled probe to single stranded target DNA sequences. Merged sample droplet 1616 may be repeatedly transported back and forth using droplet operations between temperature control zones 1612b and 1612a for PCR amplification of target DNA.

In another step, FIG. 16D shows merged sample droplet 1618 transported using droplet operations to droplet operations electrode 1610D, which is within the range of detection spot 1614. An imaging device (e.g., fluorimeter, not shown), arranged in proximity of detection spot 1614, is used to capture and quantitate the amount of fluorescence in merged sample droplet 1616. Amplified nucleic acid may be detected after any number of amplification cycles (i.e., real-time or end-point). In some embodiments, a droplet containing amplified nucleic acid may be transported for further processing, e.g., RFLP SNP genotyping or pyrosequencing.

Several different fluorescent probe-based detection technologies may be used in the process of detecting an unanchored amplified target sequence on a droplet actuator. In one example, reagent droplet 1618 may include a fluorescently labeled single stranded probe, such as a HYBEACON® probe (available from Evogen, Inc., Kansas City, Mo.). HYBEACON® probes contain two fluorophores that display a large increase in fluorescence upon hybridization to a target DNA sequence. Because unbound HYBEACON® probes display minimal fluorescence, they may be used in hybridization reactions where amplified DNA targets are not immobilized on magnetically responsive beads. HYBEACON® probes may be used for real-time or endpoint PCR analysis.

In another example, reagent droplet 1618 may include two fluorescently labeled probes for a two probe FRET detection protocol. One fluorescently labeled probe is an anchor probe and the other fluorescently labeled probe is a detection probe. The anchor probe and the detection probe hybridize to the target sequence immediately contiguous to one another. When both probes are hybridized to the target sequence in this configuration, the fluorescence emission energy of the anchor probe is transferred to the detection probe which produces a fluorescent signal. A FRET type detection protocol may be used for real-time or endpoint PCR analysis.

In another example, reagent droplet 1618 may include a single oligonucleotide probe that is labeled with a fluorescent reporter molecule and a quencher molecule (i.e., internal FRET probe). The internal FRET probe may, for example, be a TAQMAN® probe. TAQMAN® probes include a fluorophore covalently attached to the 5′-end of the oligonucleotide probe and a quencher at the 3′-end. As long as the fluorophore and the quencher are in proximity, quenching inhibits any fluorescence signals. The exonuclease activity (5′ to 3′) of Taq polymerase is used to degrade the internal FRET probe bound to a specific target sequence. Upon separation of the fluorescent reporter molecule from the quencher molecule, a fluorescent signal is generated that is proportional to the concentration of the target DNA. TAQMAN® probes may be used for real-time or endpoint PCR analysis.

Internal FRET probes, such as TAQMAN® probes, may also be used for the allele-specific detection of a SNP region of interest. In this example, two different internal FRET probes which hybridize to each complementary allele are used. For example, probe is specific for allele 1. Probe 2 is specific for the complementary allele 2. Single base mismatched probes (e.g., probe 1 and allele 2; probe 2 and allele 1) do not hybridize at the annealing temperature and consequently are not digested by the exonuclease activity of Taq polymerase. Because the mismatched probe is not digested, a fluorescent signal is not produced.

In another example, reagent droplet 1618 may include an internal FRET probe such as a molecular beacon probe. Molecular beacon probes are hairpin shaped molecules with an internally quenched fluorophore. Fluorescence is restored when the molecular beacon probe binds to a target nucleic acid sequence. Molecular beacons may be used for real-time or end-point PCR analysis.

In another example, reagent droplet 1618 may include a primer that is covalently bound to a probe, such as a Scorpion probe that also contains a fluorophore and a quencher. During PCR amplification, the primer hybridizes to the target and is extended by DNA polymerase. As the primer is extended, the fluorophore and the quencher are separated and a fluorescent signal is produced. Scorpion probes may be used for real-time PCR analysis.

In another example, reagent droplet 1618 may include primers, 3′-blocked sequence specific probe and a double stranded DNA binding dye (e.g., LCGreen dye, such as LCGREEN® Plus available from Idaho Technology Inc., Salt. Lake City, Utah) for DNA high resolution melting point analysis of amplified DNA sequences (e.g., pathogen DNA, SNP region of interest). In this example, forward and reverse primers are selected to generate a short PCR amplicon, e.g., about 50-100 bp. The concentration of the primers in reagent droplet 1618 may, for example, be provided at a ratio of about 1:5 to 1:10 for asymmetric PCR. LCGreen dye produces a fluorescent signal when bound to double-stranded DNA. The 3′-blocked probe is specific for amplified sequences and is not extended during PCR cycling. After a sufficient number of amplification cycles, a melting curve analysis is performed. The temperature in temperature control zone 1612b may be adjusted for melting curve analysis. For example, the temperature in temperature control zone 1612b may be adjusted by increasing the temperature at a rate of about 0.3° C./second in a range of about 50° C. to 95° C. As the temperature is increased, the amount of fluorescence in merged sample droplet 1616 is determined (i.e., melting is associated with a decrease in fluorescence signal). Melting point analysis may be used for end-point detection.

7.3.3 Allele Specific Primer Extension

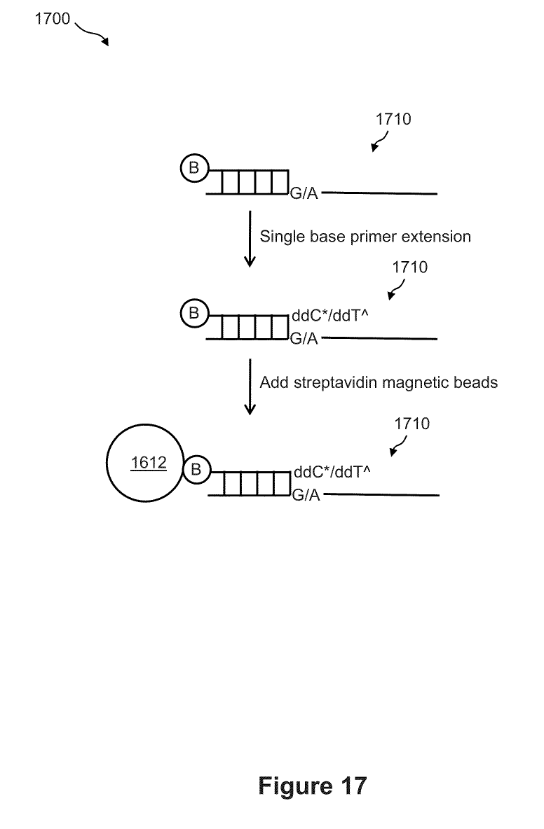

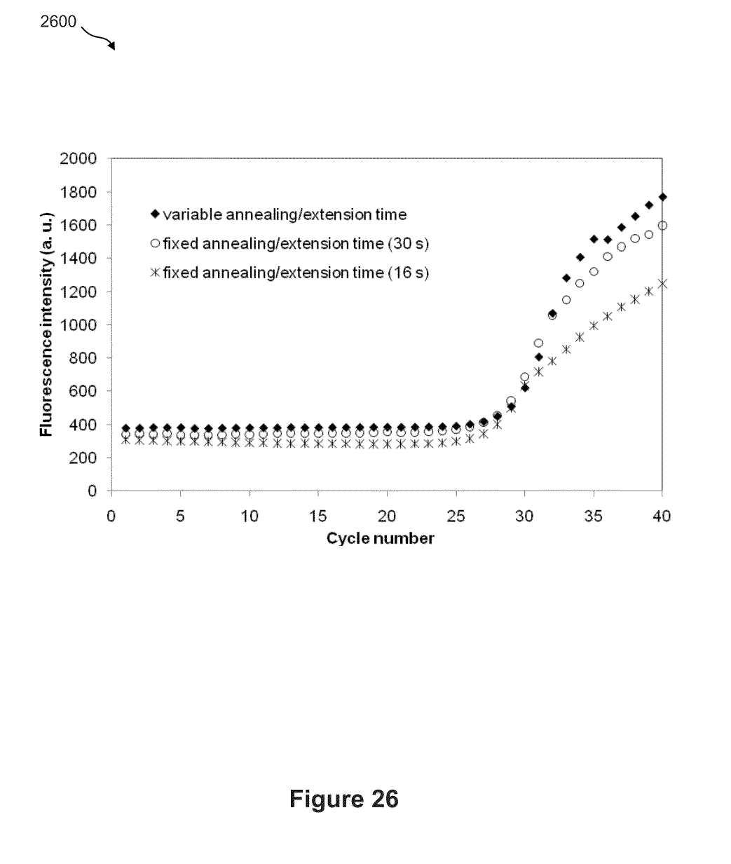

Allele specific primer extension may also be used for detection of SNPs and pathogen nucleic acid. FIG. 17 illustrates an example of a process 1700 of allele specific primer extension. In this embodiment, one or more fluorescently labeled nucleotides are incorporated during primer extension of a nucleic acid target. In one example, a SNP region 1710 (G/A) may be interrogated using a biotinylated primer (B) that is complementary to the target sequence and single base primer extension. A fluorescent tag is incorporated into the extended DNA. In this example, one allele is distinguished by incorporation of ddC* that is labeled with a first fluorophore (*) and the other allele is distinguished by incorporation of ddT̂ that is labeled with a second fluorophore (̂). To facilitate detection of the fluorescent signals, the labeled target sequences may be bound to streptavidin coated magnetically responsive beads 1712. A head washing protocol, such as the bead washing protocol described in reference to FIGS. 2F and 26, may be used to remove excess unincorporated fluorescent ddC* and ddT̂. An imaging device (e.g., fluorimeter, not shown), may be used to capture and quantitate the amount of fluorescence from a fluorophore (ddC*) and the amount of fluorescence from a second fluorophore (ddT̂) anchored on beads 1712.

7.3.4 Examples of Restriction Endonuclease-Based SNP Genotyping

7.3.4.1 Medical Diagnostics and Pharmacogenetics

Because of the flexibility and programmability of the digital microfluidics platform, multiplexed assays for two or more genes and/or alleles may be readily performed, in addition, two or more different types of assays, such as PCR, restriction endonuclease cleavage, and allele-specific PCR may be readily performed sequentially and/or simultaneously on a droplet actuator.

Examples of PCR-RFLP analysis for a specific disease and/or risk are shown in Table 4. Examples of PCR-RFLP analysis for evaluation of risk for an adverse drug event are shown in Table 5.

TABLE 4
Examples of medical diagnostic applications
of PCR-RFLP analysis

| Disease | Gene | SNP | Enzyme |
|---|---|---|---|
| Sickle Cell Anemia | β-globin¹⁵ | GAG > GTG | DdeI |
| Ischemic Heart | *Apolipoprotein E4 | | |
| Disease | allele (APOE)¹⁶ | | |
| | E2 allele (position A) | CGC > TGC | HhaI |
| | E3 allele (position A) | CGC > TGC | HhaI |
| | E2 allele (position B) | CGC > TGC | HhaI |
| | APOE promoter region | −1254T > C | AluI |
| | | −1318A > T | DpnII |
| | | −1046G > T | DpnII |
| | Lipoprotein lipase | −93T > G | ApaI |
| | (LPL)^{17,18,19} | Asp9Asn | SalI |
| | | (G > A) | |
| | | Gly188Glu | AvaII |
| | | (GGG > GAG) | |
| Type II Diabetes | Apolipoprotein B | T71I | ApaLI |
| | (APOB)²⁰ | A591V | AluI |
| | | L2712P | MvaI |
| | | R3611Q | MspI |
| | | E4154K | EcoRI |

TABLE 5
SNP analysis of Thiopurine S-Methyltransferase gene²¹

| SNP | Enzyme |
|---|---|
| 238G > C | Bsl 1 |
| 460G > A | Mwo 1 |
| 719A > G | Acc 1 |

7.3.4.2 Methicillin Resistant Staphylococcus aureus (MRSA)

Methicillin-resistant Staphylococcus aureus (MRSA) is a significant cause of healthcare- and community-associated infections, and its prevalence continues to increase. High-level resistance to methicillin is caused by the mecA gene, which encodes an alternative penicillin-binding protein, PBP 2a which has low affinity for β-lactam, antibiotics. The mecA regulon (mecA, mec1, and mecR1) is carried by a mobile genetic element designated staphylococcal cassette chromosome mec (SCCmec)^{23, 24, 25}. SCCmec also includes the ccr gene complex (ccrA and ccrB, or ccrC) and J regions (junkyard, J1, J2, and J3). The structural organization of SCCmec is J1-ccr-J2-mec-J3. SCCmec genotypes are defined by the combination of the class of mec gene complex (3 classes) with the ccr allotype (four ccrAB allotypes, ccrC). Six SCCmec types (I-VI) have been identified in S. aureus. Variations in the J regions may be used for defining SCCmec subtypes²⁵.

SCCmec typing has been established as an important component in the characterization and identification of MRSA strains. Currently, increasing numbers of community-acquired MRSA (CA-MRSA) strains are appearing that area able to cause severe infections in otherwise healthy people. CA-MRSA strains are generally SCCmec type IV or type V. Multiplex PCR assays for rapid SCCmec typing have been developed based on sequence variations in the mecA complex and/or the ccr gene complex. In one example, PCR amplification of the ccrB gene and subsequent HinfI and BsmI restriction endonuclease digestion may be used to rapidly identify four SCCmec types (I-IV), especially type IV, based on different RFLP patterns²⁴. In another example, a single multiplex PCR assay may be used for the rapid identification of all major subtypes of SCCmec type IV^{25,26}.

7.3.4.3 Influenza A Viruses

Influenza A viruses circulate worldwide and cause annual epidemics of human respiratory illness. Influenza A viruses are classified by subtype on the basis of the two main surface glycoproteins hemagglutinin (HA) and neuraminidase (NA). Different subtypes (e.g., H1N1 and H3N2) may be in circulation among human populations at different times. Influenza A viruses are further characterized into strains. Because influenza Viruses are dynamic and constantly evolving, new strains continually appear. In addition, new subtypes of influenza A may be introduced into the human population from animal sources (e.g., avian, swine) or by genetic reassortment i.e., mixing, of human and animal influenza A genes to create a new subtype.

PCR-RFLP methodologies for genotyping influenza A viruses may be based on polymorphisms in HA²⁷ and/or NA coding regions or internal viral gene sequences²⁸. In one example, two H3N2 influenza viral strains, A/LA/1/87 and A/Sydney/5/97 may be distinguished by HpaI digestion of a PCR amplicon²⁷. In this example, the A/LA/1/87 PCR amplicon has a single HpaI restriction site that is absent in the A/Sydney/5/97 PCR amplicon.

In another example, a genotyping strategy may include RFLP analysis of one or more internal gene sequences of influenza A viruses. A genotyping strategy for distinguishing H1N1, H3N2, and H5N1 subtypes is shown in Table 6. In this example, conserved primer sites were identified for each of 6 internal influenza A virus genes²⁸. The sequences of each PCR amplicon were then analyzed to identify a single, unique restriction endonuclease site for each viral subtype. For example, in the viral NS gene amplicon, a single DraI site is unique to the H1N1 viral subtype, a single XbaI site is unique to the H3N2 subtype, and a single BsrBI site is unique to the H5N1 subtype. This genotyping strategy may be readily updated (e.g., PCR primer sequences and/or restriction enzymes) to compensate for changes in viral subtypes in current circulation.

TABLE 6
Genotyping H1N1, H3N2, and H5N1 Influenza A viruses²⁸

| | |
|---|---|
| Amplicon | Subtype-specific restriction enzyme |

| Gene | size (bp) | H1N1 | H3N2 | H5N1 |
|---|---|---|---|---|
| NS | 890 | DraI | XbaI | BsrBI |
| M | 847 | HindIII | ScaI | AvaII |
| NP | 1506 | HaeII | SacII | BamHI |
| PA | 773 | BbsI | EcoNI | NsiI |
| PB1 | 715 | ScaI | XmnI | BsrBI |
| PB2 | 1007 | EcoRV | BstZ171 | BglII |

7.3.4.4 SNP Database and SNP Analysis Tools

The Single Nucleotide Polymorphism database (dbSNP) is a public-domain archive for a broad collection of simple genetic polymorphisms (www.ncbi.nlm.nih.gov/projects/SNP/).

A comprehensive web-based application, SNP Cutter, has been created to simplify the PCR-RFLP assay design²² (http://bioinfo.bsd.uchicago.edu/SNP_cutter.htm). Starting from SNP sequence data preparation, SNP Cutter performs batch and automated assay design for PCR-RFLP, using pre-selected or customizable list of restriction enzymes.

7.3.5 Preparation of Genomic DNA on a Droplet Actuator

In another embodiment, genomic DNA from a biological sample may be prepared on a droplet actuator. In one example, genomic DNA, such as genomic DNA from blood cells, may be prepared using, for example, DYNABEADS® beads. A droplet including lysis buffer and magnetically responsive DYNABEADS® beads may be combined using droplet operations with a blood sample to yield a lysed sample droplet in which released DNA is bound to the DYNABEADS® beads. The DNA capture droplet may be transported using droplet operations into the presence of a magnet and washed using a merge-and-split wash protocol to remove unbound material, yielding a washed bead-containing droplet substantially lacking in unbound material. A droplet including resuspension buffer may be merged with the washed bead-containing droplet, yielding a DNA/bead-containing droplet. The DNA/bead-containing droplet may be transported using droplet operations into a thermal zone to promote release of DNA from the Dynabeads, e.g., by heating to approximately 65° C. The eluted DNA contained in the droplet surrounding the DYNABEADS® beads may then be transported away from the DYNABEADS® beads for further processing on the droplet actuator, e.g., for execution of a droplet based PCR amplification protocol, restriction endonuclease detection of a SNP region of interest, and/or pyrosequencing.

In another example, genomic DNA may be prepared from a pathogenic organism, such as the fungal pathogen Trichophyton tonsurans (ringworm of the scalp). In this example, genomic DNA may be isolated on a droplet actuator directly from a scalp swab. The expected number of Trichophyton tonsurans on a scalp swab is about 100-500 organisms.

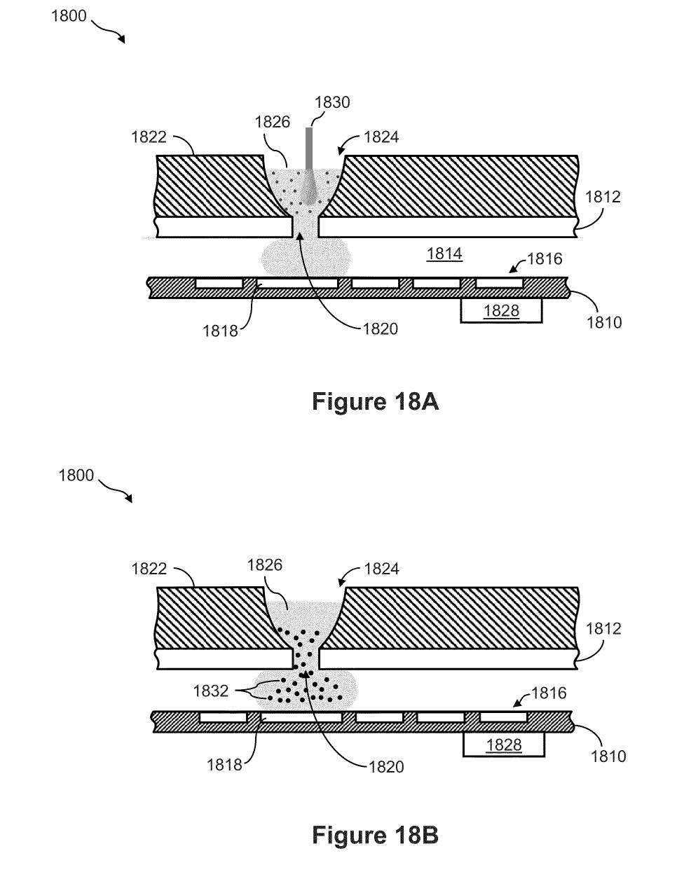

FIGS. 18A and 18B illustrate side views of an example of a portion of a droplet actuator 1800 and show a process of integrating sample preparation from a scalp swab on a droplet actuator. Droplet actuator 1800 may include a bottom substrate 1810 that is separated from a top substrate 1812 by a gap 1814. An arrangement of droplet operations electrodes 1816 (e.g., electrowetting electrodes) and a dispensing electrode 1818 may be disposed on bottom substrate 1810. Droplet operations are conducted atop droplet operations electrodes 1816 on a droplet operations surface. An opening 1820 may be provided within top substrate 1812. Opening 1820 is substantially aligned with dispensing electrode 1818. A substrate 1822 may be disposed atop top substrate 1812. Substrate 1822 may include a well 1824 which is suitable for delivering liquid through opening 1820 and into gap 1814. Well 1824 contains a quantity of fluid 1826. Fluid 1826 may, for example, be a lysis solution. A magnet 1828 is arranged in close proximity to droplet operations electrodes 1816, in particular, magnet 1828 is arranged such that a certain droplet operations electrodes 1816 (e.g., droplet operations electrode 1816M) is within the magnetic field thereof. Magnet 1828 may, for example, be a permanent magnet or an electromagnet.

An example of a process of preparing a DNA sample from a biological sample, such as a scalp swab may include, but is not limited to, the following steps.

In one step, FIG. 18A shows a sample collection and lysis protocol in which a swab 1830 is used to collect a sample, such as a fungal sample from the scalp of a subject. Swab 1830 is then placed into well 1824 that contains fluid 1826 in order to resuspend the sample and release the cells into the solution. One or more different lysing reagents may be added to fluid 1826 and incubated at one or more different temperatures to yield a lysed cell solution that contains released DNA. In a specific example, swab 1830 is incubated in 200 μL of fluid 1826 (0.05 M sodium hydroxide) and heated at 95° C. for 10 minutes. Spheroplasts are produced by adding 500 μL of a solution containing lyticase (10 U/mL), 50 mM Tris HCl (pH 7.5), 20 mM EDTA, 28 mM mercaptoethanol to fluid 1826 and incubating at 37° C. for 30 minutes. The spheroplasts are lysed by the addition of proteinase K to fluid 1826 and incubating at 55° C. for 15 minutes to release genomic DNA.

In another step, FIG. 18B shows a DNA recovery process in which a quantity of magnetically responsive beads 1832, such as DYNABEADS® beads, are added to the lysed cell solution. The lysed cell solution with magnetically responsive beads 1832 therein is incubated for a sufficient period of time for released DNA to bind magnetically responsive beads 1832. The lysed solution with magnetically responsive beads 1832 therein is then loaded onto dispensing electrode 1818. One or more DNA capture droplets (not shown) may be transported using droplet operations into the presence of magnet 1828 and washed using a merge-and-split wash protocol to remove unbound material, yielding a washed bead-containing droplet substantially lacking in unbound material (not shown). The purified DNA is then eluted from magnetically responsive beads 1832 with 10 mM Tris HCl, 1 mM EDTA, pH 7.4. The eluted DNA contained in the droplet surrounding the DYNABEADS® beads may then be transported away from the DYNABEADS® beads for further processing on the droplet actuator, e.g., for execution of a droplet based PCR amplification protocol and pyrosequencing.

7.3.6 Integrated Digital Microfluidic PCR and Pyrosequencing

Digital microfluidic pyrosequencing combines PCR amplification of target sequences and sequencing on a single droplet actuator. Flow-through PCR may be performed on the droplet actuator using established protocols that include optimum cycling parameters and concentration of reagents including Taq polymerase, buffers and primers. One example of a typical PCR protocol is to dispense one 450 nL droplet of sample and one 450 nL of PCR reaction mixture and merge the droplets using droplet operations. The merged droplet is then thermocycled by transporting the droplet between two thermal zones (e.g., 95° C. zone and a 55° C. zone). The centers of the two thermal zones may be separated by 16 electrodes. The transport rate of the droplet may be up to 25 Hz (i.e., electrodes per second).

PCR primer concentration may be selected to provide a primer:amplicon ratio of about 5:>1, or about 5:>1.5, or about 1:1, or about 1:2, or about 1:3. In one example, the PCR primers may be selected to amplify the ITS region of the ribosomal DNA gene which has been shown to be specific for the identification of the fungal pathogen, Trichophyton tonsurans. A quality control check may be incorporated to insure that a PCR product has been synthesized prior to initiation of a pyrosequencing protocol. In one example, the quality control check may be performed as an end-point assay or a real-time assay using a generic fluorescent indicator, such as EVAGREEN® dye. In another example, the quality control check may use a target specific probe, such as TAQMAN® probe.

To provide a platform for digital microfluidic pyrosequencing, the amplified DNA template is immobilized onto magnetically responsive beads. In one embodiment, PCR amplicons may be formed using primers covalently bound to magnetically responsive beads. In another embodiment, biotinylated PCR amplicons may be immobilized on magnetically responsive beads through formation of a biotin-streptavidin binding complex. In this example, one of the PCR primers may be a 5′-biotinylated primer to provide a ready method for anchoring the sequencing template DNA strand to magnetically responsive heads, such as streptavidin coated Dynal magnetic beads (2.8 μM diameter). The quantity of DNA template immobilized on the streptavidin coated beads may be maximized by limiting the concentration of biotinylated primer used for PCR amplification (i.e., substantially all primer is incorporated into the amplicon). In another example, PCR amplicons may be purified using magnetically responsive beads (e.g., DYNABEADS® beads) to remove excess biotinylated primers.

7.3.6.1 Template Preparation for Pyrosequencing

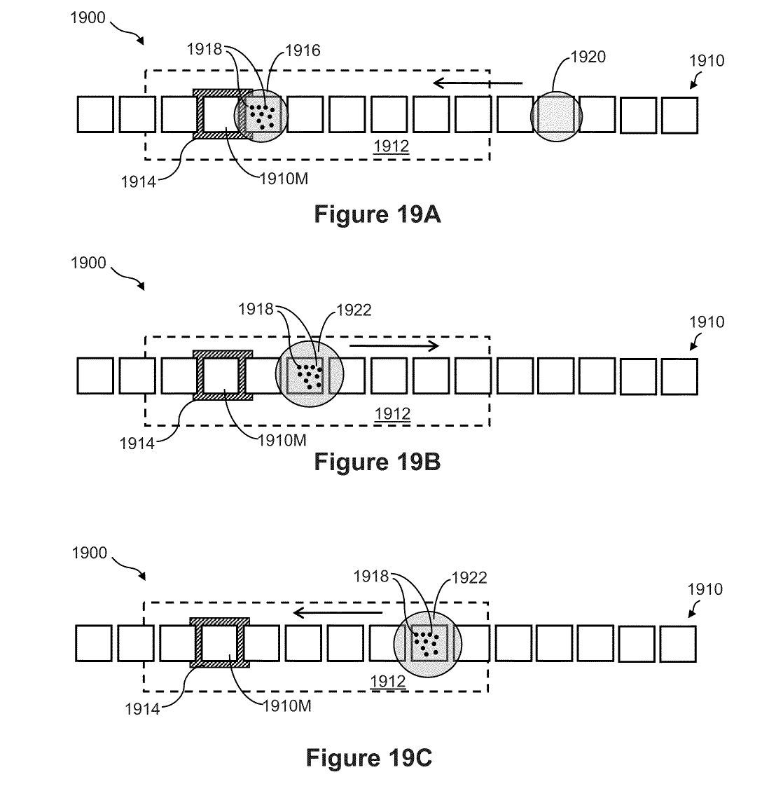

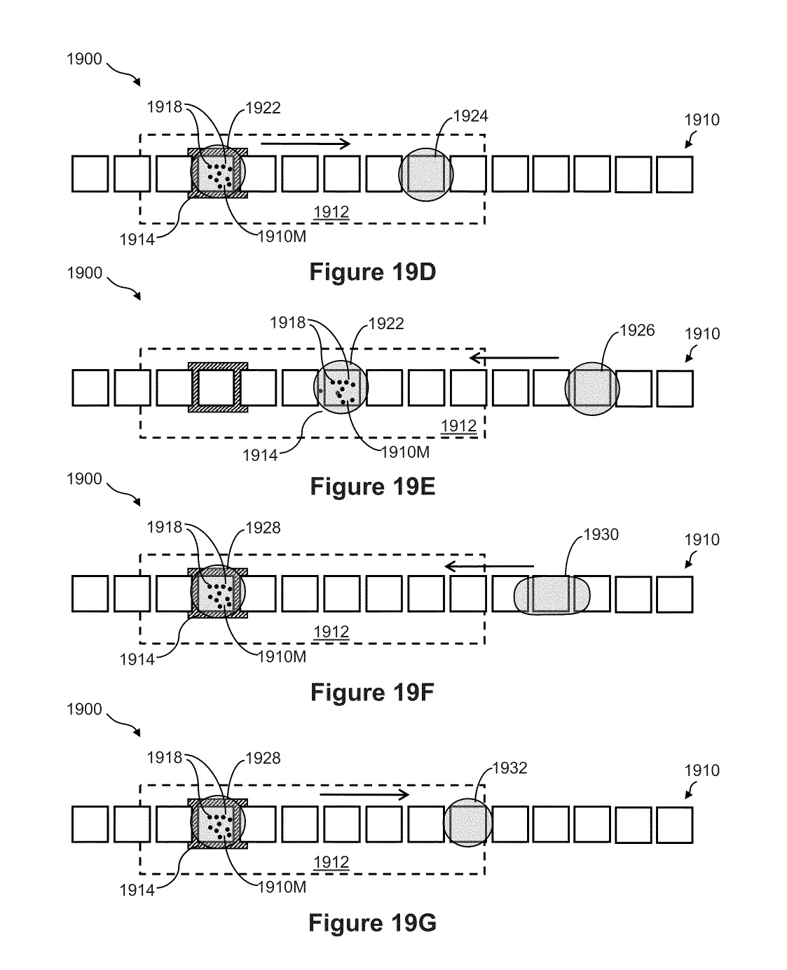

FIGS. 19A through 19G illustrate top views of an example of a portion of an electrode arrangement 1900 of a droplet actuator (not shown) and show a process of preparing a single stranded template for pyrosequencing on a droplet actuator. The method of the invention of FIGS. 19A through 19G is an example of a sample preparation protocol in which biotinylated amplified DNA sample is immobilized on streptavidin coated magnetically responsive beads and single stranded sequencing template prepared by alkali denaturation.

Electrode arrangement 1900 may include an arrangement of droplet operations electrodes 1910 (e.g., electrowetting electrodes) that is configured for preparation of a DNA template for sequencing. Droplet operations are conducted atop droplet operations electrodes 1910 on a droplet operations surface. A temperature control zone 1912 may be associated with electrode arrangement 1900. Thermal control elements (not shown) control the temperature of filler fluid (not shown) in vicinity of temperature control zone 1912. For example, temperature control zone 1912 may be heated to about 65° C. for a defined period of time and then adjusted to a different temperature for subsequent droplet operations.

A magnet 1914 is provided in proximity to temperature control zone 1912 for retaining a quantity of magnetically responsive beads. In particular, magnet 1914 is arranged such that a certain droplet operations electrode 1910 (e.g., droplet operations electrode 1910M) is within the magnetic field thereof. Magnet 1914 may, for example, be a permanent magnet or an electromagnet.

An example of a process of preparing a single stranded template for pyrosequencing on a droplet actuator may include, but is not limited to, the following steps.

In one step, FIG. 19A shows a 2× bead droplet 1916 positioned at a certain droplet operations electrode 1910M in proximity of the magnetic field of magnet 1914. “X” refers to the number of unit-sized droplets contained in the volume (e.g., X=350 nL). Bead droplet 1916 includes a binding buffer and a quantity of streptavidin-coated magnetically responsive beads 1918. In one example, 2× bead droplet 1916 may be formed by dispensing and combining using droplet operations two 1× bead droplets 1916 with magnetically responsive beads 1918 (e.g., 10 mg/mL) therein. A 2×DNA droplet 1920 is positioned at a certain droplet operations electrode 1910 in proximity of 2× bead droplet 1916. DNA droplet 1920 includes biotinylated amplified DNA. In one example, 2×DNA droplet 1920 may be formed by dispensing and combining using droplet operations two 1×DNA (e.g., about 100 ng/μL) droplets 1920.

DNA droplet 1920 is merged using droplet operations with bead droplet 1916 within temperature control zone 1912 to yield a 4× binding droplet 1922. Temperature control zone 1912 is heated to an incubation temperature of 65° C.

In other steps, FIGS. 19B and 19C show an incubation process, in which binding droplet 1922 is repeatedly transported back and forth (indirection indicated by arrows) using droplet operations within temperature control zone 1912 for a period of time (e.g., 15 minutes) sufficient for formation of biotin-streptavidin complexes. The biotinylated PCR amplicons are immobilized on magnetically responsive beads 1918 through formation of biotin-streptavidin complexes. After the incubation period, the temperature of temperature control zone 1912 is adjusted to ambient temperature.

In other steps, FIGS. 19D and 19E show a denaturation process, in which a 2× supernatant droplet 1924 is split off using droplet operations from binding droplet 1922. Binding droplet 1922 with magnetically responsive beads 1918 therein is now a 2× droplet. Supernatant droplet 1924 is transported away from temperature control zone 1912. Binding droplet 1922 is washed one time using a merge and split protocol with a 2× reagent droplet 1926 that contains a denaturing solution (e.g., 0.5 M sodium hydroxide (NaOH)). After washing, the 2× binding droplet is merged with a second 2× reagent droplet 1926 (0.5 M NaOH). The 4× merged droplet is incubated at ambient temperature as described in reference to FIGS. 19B and 19C. After a period of time sufficient to denature DNA (e.g., about 1 minute or about 45 seconds or about 30 seconds), the merged 4× droplet is split using droplet operations to yield a 2× single stranded DNA (ssDNA) droplet 1928.

In other steps, FIGS. 19F and 19G show a buffer exchange process, in which two wash cycles (i.e., bead washing protocols) are used to prepare ssDNA droplet 1928 for primer annealing. ssDNA droplet 1928 that has magnetically responsive beads 1918 therein is transported using droplet operations to droplet operations electrode 1910M (i.e., into the magnetic field of magnet 1914). A first bead washing protocol is used to exchange the denaturation solution in ssDNA droplet 1928 with a wash buffer that does not contain any precipitate forming cations. A 2× wash buffer droplet 1930 is transported along droplet operations electrodes 1910 and combined using droplet operations with ssDNA droplet 1928, which is retained at droplet operations electrode 1910M to form a merged droplet The merged droplet is divided using droplet operations into a 2× ssDNA droplet 1928 that has magnetically responsive beads 1918 therein and a 2× supernatant droplet 1932 without a substantial amount of beads. In one example, ssDNA droplet 1928 is washed twice using 2× wash buffer droplets. This step is used to prevent clumping of magnetically responsive beads 1918 and precipitation of metallic hydroxides during subsequent droplet operations.

A second washing protocol is used to exchange the wash buffer in ssDNA droplet 1928 with an annealing buffer. In one example, ssDNA droplet 1928 is washed twice using 2× annealing buffer droplets.

In another step, the 2× ssDNA droplet 1928 is combined with a 2× primer droplet (not shown) to yield a 4× ssDNA/primer droplet and incubated in a process as described in reference to FIGS. 19B and 19C for a period of time (e.g., about 1 minute) sufficient annealing of primer to ssDNA template. The 2× primer droplet contains sequencing primer diluted, for example, to 10 μM in water or in 0.01% TWEEN® 20 solution (available from Promega Corporation, Madison, Wis.). TWEEN® 20 is also known generically as Polysorbate 20. The temperature of temperature control zone 1912 is adjusted to about 80° C., which is a temperature sufficient for primer annealing. After the incubation period, the 4× ssDNA/primer droplet is split using droplet operations to yield a 2× ssDNA/primer droplet 1928.

In another step, a bead washing protocol, such as the bead washing protocol described in reference to FIGS. 19F and 19G is used to remove excess unbound primers from ssDNA/primer droplet 1928. In this step, wash buffer droplet 1930 contains a buffer suitable for pyrosequencing. In one example, ssDNA/primer droplet 1928 is washed twice using 2× pyrosequencing buffer droplets.

In another step, the 2× ssDNA/primer droplet (in pyrosequencing buffer) is combined with a 2× droplet that contains SSB protein (about 5× concentration) and incubated in a process as described in reference to FIGS. 19B and 19C for about 1 minute. The temperature of temperature control zone 1912 is adjusted ambient temperature. 2× ssDNA/primer droplet 1928 in pyrosequencing buffer is ready for sequencing.

In another embodiment, PCR amplicons may be formed using primers covalently bound to magnetically responsive beads, in this example, PCR amplicons are already bound to magnetically responsive beads and template preparation may begin with DNA denaturation as described in reference to FIGS. 19D and 19E.

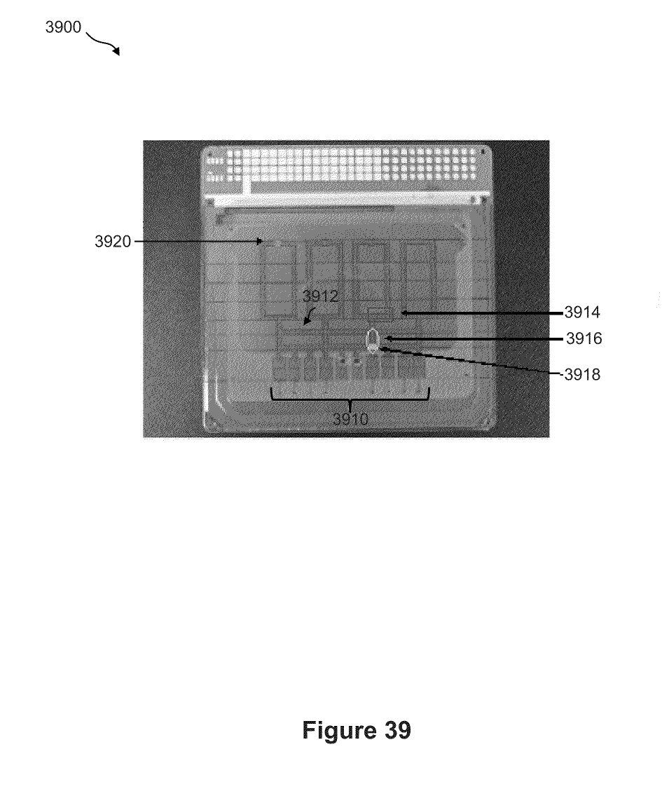

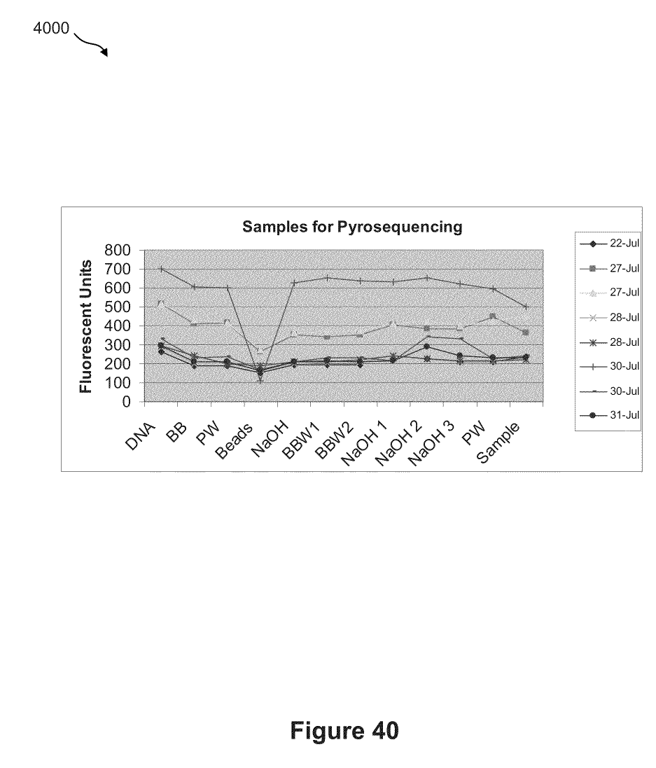

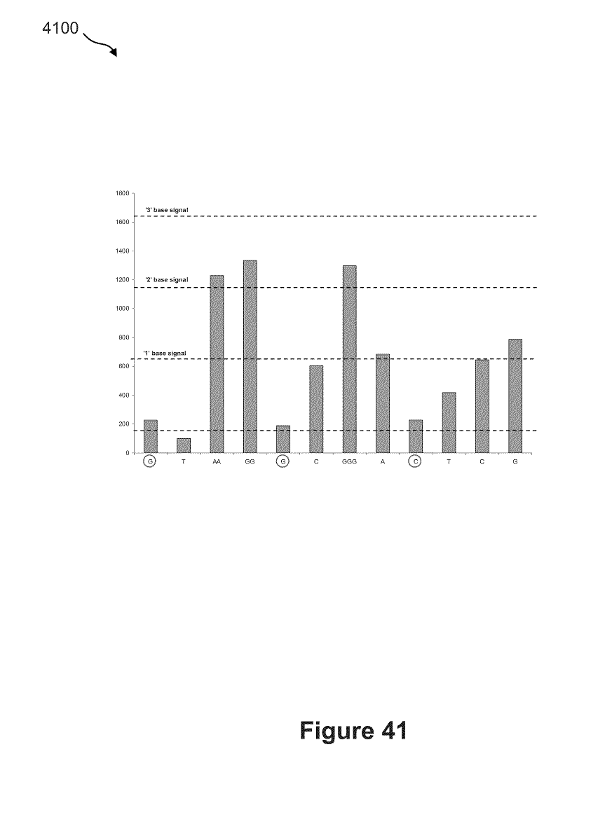

A specific example of translation of an on-bench protocol for template preparation for pyrosequencing on a droplet actuator is described in reference to FIGS. 39 and 40.

7.3.6.2 Pyrosequencing

Pyrosequencing may be performed on the droplet actuator using established sequencing protocols. Pyrosequencing may be optimized on the droplet actuator to provide sufficient read length and accuracy performance. Sequencing primers may be designed to generate DNA sequence over an appropriate region to achieve accurate identification of a target sequence fungal identification).

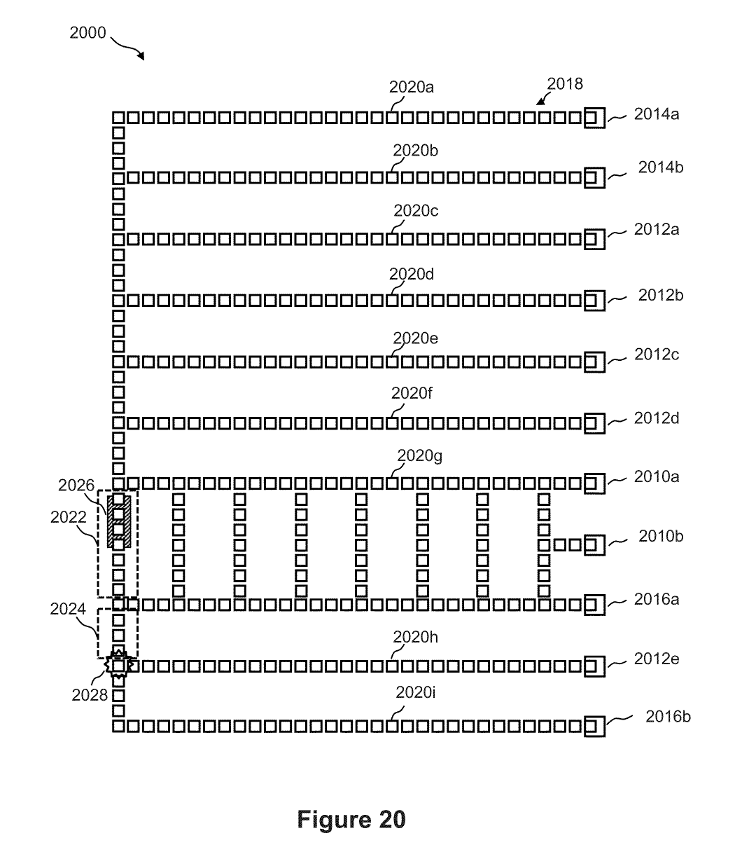

FIG. 20 illustrates a top view of an example of a portion of an electrode arrangement 2000 of a droplet actuator (not shown) that is configured for pyrosequencing on a droplet actuator. Pyrosequencing is a sequencing-by-synthesis method in which a primed DNA template strand is sequentially exposed to one of four nucleotides in the presence of DNA polymerase. If the added nucleotide is complementary to the next unpaired base, then it is incorporated by the polymerase and inorganic pyrophosphate is released. Real-time detection of pyrophosphate occurs through an enzymatic cascade in which pyrophosphate is converted by a second enzyme, sulfurylase, to ATP which provides energy for a third enzyme, luciferase, to oxidize luciferin and generate a light signal. The amount of light generated is proportional to the number of adjacent unpaired bases complementary to the added nucleotide.

Electrode arrangement 2000 includes multiple dispensing electrodes, which may, for example, be allocated as sample dispensing electrodes 2010a and 2010b for dispensing sample fluids (e.g., DNA immobilized on magnetically responsive beads); reagent dispensing electrodes 2012, i.e., reagent dispensing electrodes 2012a through 2012e, for dispensing different reagent fluids (e.g., dATPαs, dCTP, dGTP, dTTP, enzyme mix); wash buffer dispensing electrodes 2014a and 2014b for dispensing wash buffer fluids; and waste collection electrodes 2016a and 2016b for receiving spent reaction droplets. Sample dispensing electrodes 2010, reagent dispensing electrodes 2012, wash buffer dispensing electrodes 2014, and waste collection electrodes 2016 are interconnected through an arrangement, such as a path or array, of droplet operations electrodes 2018. A path of droplet operations electrodes 2018 extending from each dispensing and collection electrodes forms dedicated electrode lanes 2020, i.e., dedicated electrode lanes 2020a through 2020i. Dedicated electrode lanes provide transport of nucleotide base droplets to a reactor lane. The use of dedicated lanes for nucleotide base droplets minimizes cross-contamination among nucleotides. A dedicated electrode lane provides transport of enzyme mix directly onto the detection electrode. Using a dedicated electrode lane for enzyme mix reduces enzyme deposition on the wash lanes. Reduction of enzyme contamination permits the start of the sequencing reaction to be precisely controlled.

Electrode arrangement 2000 may include a washing zone 2022 and a reaction zone 2024. A magnet 2026 is located underneath wash lane 2022. Magnet 2026 may be embedded within the deck that holds the droplet actuator when the droplet actuator is mounted on the instrument (not shown). Magnet 2026 is positioned in a manner which ensures spatial immobilization of nucleic acid-attached beads during washing between the base additions. Mixing may be performed in reaction zone 2024 away from the magnet. The positioning of the wash buffer dispensing electrodes 2014 and waste collection electrodes 2016 improves washing efficiency and reduces time spent in washing. A detection spot 2028 is positioned in proximity of reaction zone 2024.

An example of a three-enzyme pyrosequencing protocol is as follows. A PCR amplified DNA template hybridized to a sequencing primer may be coupled to magnetically responsive beads. A droplet of the beads suspended in wash buffer may be combined with a droplet of one of the four nucleotides mixed with APS and luciferin in wash buffer. A droplet containing all three enzymes (DNA polymerase, ATP sulfurylase and luciferase) may be combined with the bead and nucleotide-containing droplet. The resulting droplet may be mixed and transported to the detector location. Incorporation of the nucleotide may be detected as a luminescent signal proportional to the number of adjacent bases incorporated into the strand being synthesized, or as a background signal for a non-incorporated (mismatch) nucleotide. After the reaction is complete, the beads may be transported to the magnet and washed. Washing may be accomplished by addition and removal of wash buffer while retaining substantially all beads in the droplet. This entire sequence constitutes one full pyrosequencing cycle which may be repeated multiple times with a user defined sequence of base additions.

In a specific example, a PCR amplified DNA template hybridized to a sequencing primer may be coupled to 2.8 μM diameter magnetically responsive beads. A 2× (800) nL droplet of the beads suspended in wash buffer may be combined with a 1× (400 nL) droplet of one of the four nucleotides mixed with APS and luciferin in wash buffer. A 1× (400 nL) droplet containing all three enzymes (DNA polymerase, ATP sulfurylase and luciferase) may be combined with the beads and nucleotides resulting in a 4× (1600 nL) reaction volume. The 4× droplet may be mixed and transported to the detector location. Incorporation of the nucleotide may be detected as a luminescent signal proportional to the number of adjacent bases incorporated into the strand being synthesized, or as a background signal for a non-incorporated (mismatch) nucleotide. After the reaction is complete the beads may be transported to the magnet and washed by addition and removal of wash buffer finally resulting in the 1600 nL of reaction mix being replaced by 800 nL of fresh wash buffer while essentially all of the beads may be retained in the droplet. This entire sequence constituted one full pyrosequencing cycle which may be repeated multiple times with a user defined sequence of base additions, in the above protocol, “X” refers to the number of unit-sized droplets contained in the volume. A unit droplet is approximately the smallest volume that can be handled based on the size of the individual electrodes.

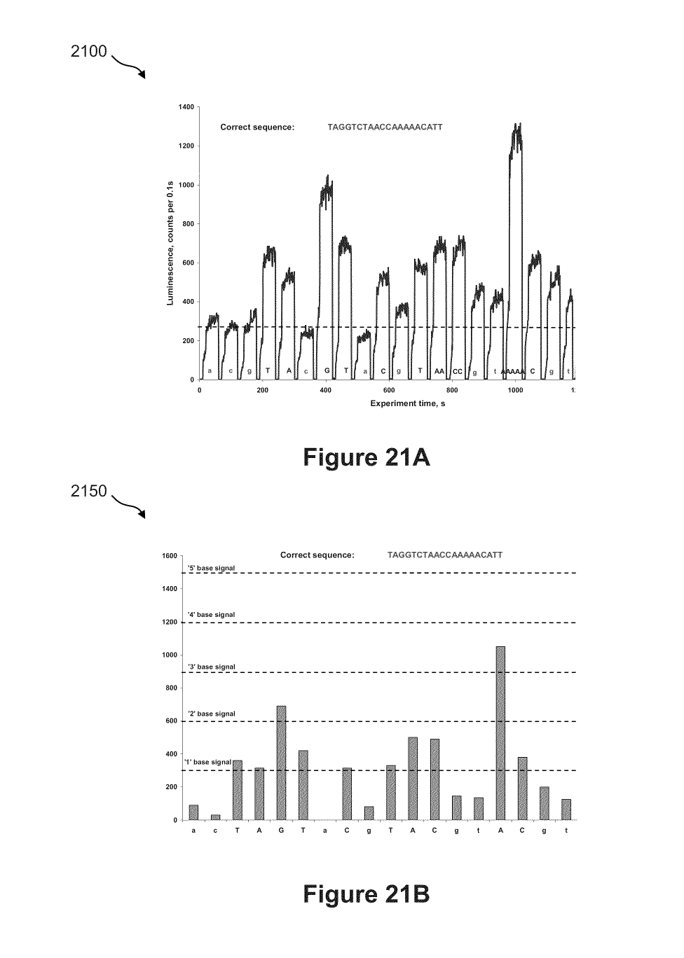

FIGS. 21A and 21B show an example of a pyrogram 2100 and a histogram 2150, respectively, of on-actuator pyrosequencing results of 17-bp sequenced on a 211-bp long C. albicans DNA template using cyclic nucleotide dispensing. FIG. 21A shows pyrogram 2100, which is the actual pyrogram output of the experiment showing each peak. A total detection time of 60 sec was used for each cycle alternating between 10 sec of mixing and 10 sec of detection. Non-detecting time intervals are removed from the figure for easy visualization. FIG. 21B shows histogram 2150 with the peak heights of the signal corresponding to different nucleotide additions.

Nucleic acids were sequenced using a eye nucleotide dispensing strategy (i.e., A,C,G,T repeated in that order). Alternatively, for the same template, up to 20 bases were sequenced with identical results using an ordered nucleotide dispensation strategy (i.e. in order according to a reference sequence; not shown).

7.3.7 Identification of Clinically Relevant Fungi

Integrated digital microfluidic technology that combines sample preparation, PCR amplification and pyrosequencing may be used to accurately and rapidly identify clinically relevant fungi. In one embodiment, pure fungal cultures of clinically relevant fungi may be used to establish identification criteria (e.g., amplicon length, primer pairs, and target region). For example, six or more common yeast species, such as Candida albicans, C. glabrata, C. Krusei, C. parapsilosis, C. tropicalis, and Cryptoccocus neoformans, may be evaluated. In addition, eight or more clinically relevant mould species, such as Alternaria alternate, Aspergillus flavus, A. fumigates, A. terreus, Fusarium oxysporum complex, F. solani complex, Rhizopus oryzae, and Scedosporium apiospermun, may be evaluated.

In this example, fungal genomic DNA may be extracted on-bench using either a phenol-chloroform or commercial technique. The extracted DNA may be measured for concentration and purity using, for example, a Nanodrop™ spectrophotometer and stored at −80° C. for subsequent PCR amplification. PCR primers may, for example, be selected to amplify the ITS2 region of the ribosomal DNA gene, which has been shown to be a useful region for determining fungal species identification. Amplicons may be verified by gel electrophoresis, processed for removal of post-PCR reagents, measured spectrophotometrically for concentration and purity, and prepared for nucleic acid sequencing. Pyrosequencing may be performed using digital microfluidic protocols. A second sequencing technique, such as the Biotage PyroMark™ sequencing platform, may be used for verification.

In another embodiment, patient specimens may be analyzed directly by PCR amplification and pyrosequencing to determine the nucleic acid sequence of the resulting amplicon. For example, direct PCR testing may be used for diagnosis of tinea capitis (ringworm of the scalp). Trichophyton tonsurans, a major etiologic agent of tinea capitis, may be identified by integrated digital microfluidic PCR amplification of the ITS region of the ribosomal DNA gene and pyrosequencing. In one example, tinea capitis specimens may be cultured from scalp swabs, genomic DNA prepared and subsequently analyzed by integrated digital microfluidic PCR amplification and pyrosequencing. In another example, genomic DNA may be isolated directly on a droplet actuator from scalp swabs and analyzed using integrated digital microfluidic PCR and pyrosequencing as described in reference to FIGS. 18A through 20.

7.3.8 Multiplexed Real-Time PCR

Because of the flexibility and programmability of the digital microfluidics platform, multiplexed PCR assays may be readily performed. Rapid PCR thermocycling is performed in a closed-loop flow-through format where for each cycle the reaction droplets are cyclically transported between different temperature zones within the oil filled droplet actuator. The droplet actuator may be fabricated using low-cost PCB technology and is intended to be a single-use disposable device.

Variable cycle times (“smart” cycle time management) combined with the rapid thermocycling provided by digital microfluidics significantly reduces the PCR assay time without compromising reaction yield. The multiplexed real-time PCR system includes magnetic bead handling capability which may, for example, be applied to analyze clinical samples prepared from whole blood using a magnetic bead capture protocol.

In one embodiment, the real-time PCR system may be used to accurately and reliably detect microbial DNA in clinical specimens for the rapid diagnosis of infectious diseases (e.g., Staphylococcus aureus (MRSA), Mycoplasma pneumoniae or Candida albicans). In another embodiment, the multiplexed real-time PCR system (e.g., parallel two-plex PCR amplification of multiple DNA samples) may be used for high-throughput multiplexed PCR applications.

The reproducibility and sensitivity of the digital microfluidic PCR system of the invention provides many advantages in terms of automation, cost and time-to-result in a PCR assay. The design of the droplet actuator is highly modular enabling it to be scaled-up for high throughput applications or combined with other modules to meet application-specific demands. The flexibility and breadth of digital microfluidics combined with thermocycling and bead manipulation capability enables the integration of PCR amplification with other pre-PCR or post-PCR processes for complete “sample to answer” automation.

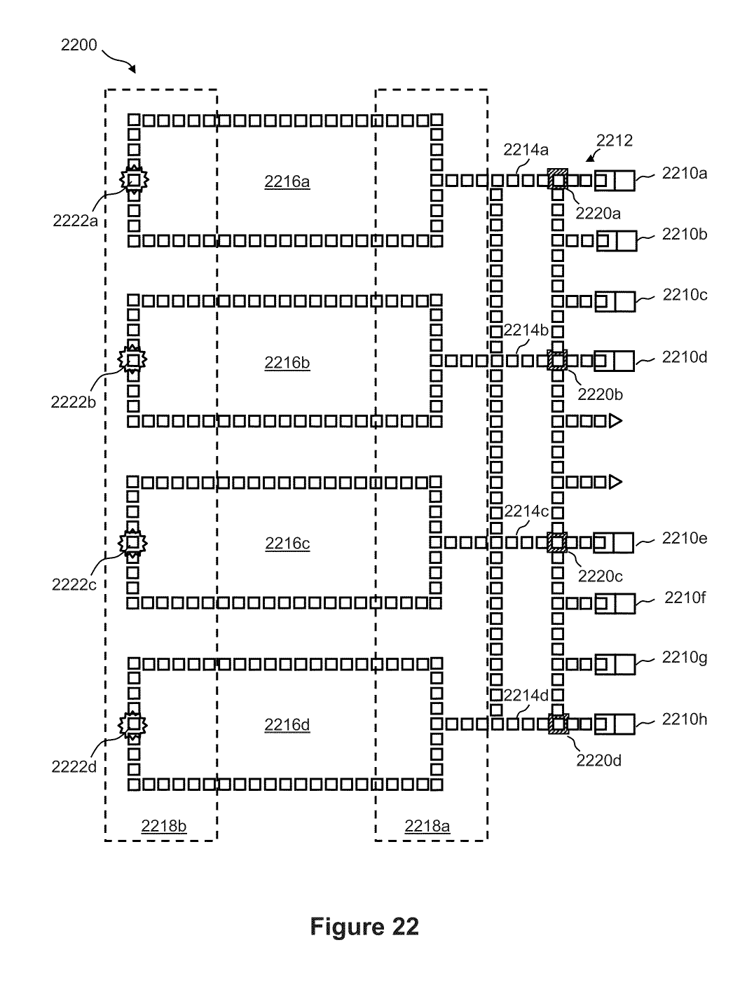

FIG. 22 illustrates a top view of an example of an electrode arrangement 2200 of a droplet actuator (not shown) that is configured for multiplexed real-time PCR Electrode arrangement 2200 includes multiple dispensing electrodes 2210, e.g., dispensing electrodes 2210a through 2210h, or dispensing different DNA sample fluids and reagent fluids (e.g., specific primer pairs, PCR buffer, dNTPs, DNA polymerase). Each dispensing electrode 2210 is aligned with a reservoir defined by the spacer of the droplet actuator. Dispensing electrodes 2210 are interconnected through an arrangement, such as a path or array, of droplet operations electrodes 2212 (e.g., electrowetting electrodes). Droplet operations are conducted atop droplet operations electrodes 2212 on a droplet operations surface. Droplet operations electrodes 2212 may, for example, have a unit electrode size of 1.1 mM×1.1 mM. The unit electrode size and gap height (e.g., about 275 μM) provide for consistent dispensing of either 300 nL droplets (i.e., 1× droplets) or 600 nL droplets (i.e., 2× droplets) in volume depending on whether the droplet is formed across one or two droplet operations electrodes 2212.

A path of droplet operations electrodes 2212 extending from dispensing electrodes 2210a, 2210d, 2210e, and 2210h form electrode lanes 2214, e.g., electrode lanes 2214a through 2214d. Electrode lanes 2214 provide transport of PCR reaction droplets to four independently controlled electrode thermocycling loops 2216, e.g., thermocycling loops 2216a through 2216d. Each thermocycling loop 2216 may circulate a single droplet or a droplet train between two temperature control zones 2218, e.g., temperature control zones 2218a and 2218h. Temperature control elements (not shown) control the temperature of filler fluid (not shown) in vicinity of temperature control zones 2218a and 2218b. For example, temperature control zone 2218a may be heated to about 95° C. (melting temperature), which is a temperature sufficient for denaturation of DNA template and primers. Temperature control zone 2218b may, for example, be heated to about 60 to 72° C., which is a temperature sufficient for annealing of primer to the single-stranded DNA template and primer extension by DNA polymerase. While two temperature control zones 2218 are shown, any number of temperature control zones 2218 may be associated with electrode arrangement 2200.

Magnets 2220 (e.g., 2220a, 2220b, 2220c, 2220d) are positioned in proximity to electrode lanes 2214 (e.g., electrode lanes 2214a, 2214b, 2214c, and 2214d), respectively, for retaining a quantity of magnetically responsive beads. Magnets 2220 may, for example, be permanent magnets or electromagnets. In one example, each magnet 2220 may be a cylindrical neodymium magnet (⅛″ D×⅛″ H, KJ Magnetics, PA).

A detection spot 2222 may be provided within each thermocycling loop 2216 of temperature control zone 2218b. For example, detection spots 2222a, 2222b, 2222c, and 2222d may be provided within thermocycling loops 2216a, 2216b, 2216c, and 2216d, respectively. The amount of amplified DNA in each thermocycling loop 2216 may be determined during each amplification cycle using a four channel fluorimeter (not shown). Each channel comprises a light emitting diode (e.g., the RL3-B2030 LED, available from Super Bright LEDs Inc. St. Louis, Mo.), a photodiode (e.g., the S2386-18K photodiode, available from Hamamatsu Corporation, Bridgewater, N.J.), and a fluorescein isothiocyanate (FITC) filter set (e.g., FITC filter sets available from Semrock, Inc., Rochester, N.Y.) along with a long-pass dichroic mirror. The fluorimeter module is mounted directly above the droplet actuator (not shown) facing temperature control zone 2218b. The excitation source (475 nM peak wavelength) produces an illumination spot that may be, for example, about 500 μM in diameter. The illumination spot is focused and centered within each detection spot 2222.

In one example of a PCR protocol, a 1×DNA sample droplet may be dispensed from dispensing electrode 2210a onto electrode lane 2214a. A 1× reagent droplet (e.g., PCR master mix) may be dispensed from dispensing electrode 2210b. The 1× reagent droplet may be combined with the 1×DNA sample droplet to yield a 2×PCR reaction droplet. The 2×PCR reaction droplet may then be transported from electrode lane 2214a using droplet operations into temperature control zone 2218a in thermocycling loop 2216a. The 2×PCR reaction droplet may then be thermocycled between temperature control zones 2218a and 2218b. In one example, an electrode switching rate from about 4 to 16 Hz may be used providing a revolution period of about 4 to 15 seconds for the 2× reaction droplet to travel through the entire thermocycling loop. At the end of each annealing/extension cycle, the 2×PCR reaction droplet may be transported to detection spot 2220a and the fluorescence of the droplet determined.

7.3.8.1 Thermal Analysis

Precise and uniform temperature control is one of the most critical requirements for a successful real-time PCR reaction. Because the PCB substrate material used in the PCR droplet actuator has relatively poor thermal conductivity, it may not be assumed that the temperature inside the droplet actuator is uniform. The steady-state temperature profile of an entire PCR droplet actuator may be simulated by finite element analysis using a commercial software package (e.g., Dassault Systèmes SolidWorks (DS SolidWorks) available from Dassault Systèmes SolidWorks Corp., Concord, Mass.). In one example, the thermal conductivity values of the PCB bottom substrate, hexadecane filler fluid, water droplet, and glass top substrate used in the thermal analysis were 0.3, 0.135, 0.64, 1.1 W·K⁻¹·m⁻¹, respectively, and the convective heat transfer coefficient (h) used for the cartridge surfaces was 10 W·K⁻¹·m⁻².

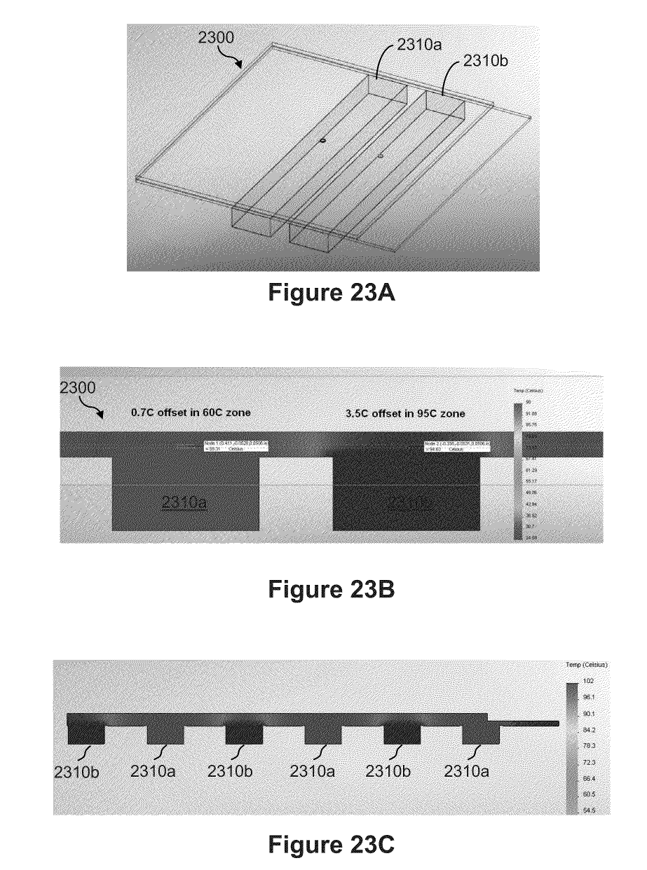

FIGS. 23A through 23C show an example of the simulation results of finite element thermal analysis of a PCR reaction. FIG. 23A shows a computer generated model (e.g., via DS SolidWorks) of an oil-filled PCR droplet actuator 2300 positioned in proximity of two temperature control elements 2310a and 2310b. Temperature control elements 2310a and 2310b control the temperature of filler fluid and define two temperature control zones used in PCR thermocycling. For example, temperature control element 2310a may be heated to about 95° C. (melting temperature), which is a temperature sufficient for denaturation of DNA template and primers. Temperature control element 2310b may, for example, be heated to about 60 to 72° C., which is a temperature sufficient for annealing of primer to the single-stranded DNA template and primer extension by DNA polymerase.

FIG. 23B shows the steady-state temperature profile of a portion of PCR droplet actuator 2300 and temperature control elements 2310. The simulation results shown in FIG. 23B indicate a steady-state temperature difference of 0.7° C. and 3.5° C. between the temperature control elements 2310a and 2310b and the center of the droplet in the 60° C. and 95° C. zones, respectively. This result was experimentally verified by inserting miniature thermocouples (e.g., thermocouples available from OMEGA Engineering, INC., Stamford, Conn.) into the droplet actuator and measuring the oil temperature in the gap between the two substrates. In all cases sufficient agreement was found between the simulations and measured values. Based on these experiments an offset was applied to the temperature control element set point during the PCR experiments to accurately control the temperature inside the droplet. According to the simulation result, the temperature differences between the top and bottom surfaces of the droplet were 0.2° C. and 0.4° C. in the 60° C. and 95° C. zones respectively, which is considered sufficient for PCR. In another embodiment, a more uniform distribution may be achieved by placing temperature control elements on both sides of the droplet actuator.

FIG. 23C shows a conceptual illustration of a high-throughput PCR system using multiple temperature control elements. Multiple temperature control elements 2310a and 2310b may be used to implement more reactions per unit of droplet actuator area. In this example, an arrangement of multiple temperature control element sets (e.g., temperature control elements 2310a and 2310b) that have smaller heater dimensions and reduced separations may be used in high-throughput applications. Based on the thermal modeling, a minimum separation required between two temperature control elements (dimensions: 6.35 mM [W]×3.18 mM [H]) to allow thermocycling between 60° C. and 95° C. in this arrangement is about 7.38 in mM. In another example, the density of temperature control elements 2310 may be further increased with the use of active cooling mechanisms and/or improvements to the thermal design.

7.3.8.2 Evaluation of Digital Microfluidic PCR

The following experimental conditions were used to evaluate the performance of the microfluidic PCR platform. The PCR mix contained 2×PCR buffer, 3 mM MgCl2, 0.2 mM each of the dNTPs, 1 μM each of the primers and 0.5 unit/ul platinum Taq polymerase (Invitrogen, CA). The mix also included either 2× EVAGREEN® dye or 1 μM TAQMAN® probe (Sigma-Aldrich, Mo.), depending on the target. The genomic DNA of methicillin-resistant Staphylococcus aureus (MRSA), Candida albicans and Mycoplasma pneumoniae was obtained from America Type Culture Collection (ATCC, MD) and prepared in biograde water with 0.1% TWEEN® 20. The sequences of the primer set and TAQMAN® probe as well as the amplicon size for each DNA template is shown in Table 7.

TABLE 7
PCR primer/probe sequences and product sizes

| | Staphylococcus aureus | Candida albicans | Mycoplasma pneumoniae |
|---|---|---|---|
| Forward | 5′-GTC AAA AAT CAT | 5′-CTG TTT GAG CGT | 5′-TTT GGT AGC TGG |
| primer | GAA CCT CAT TAC | CGT TTC-3′ | TTA CGG GAA T-3′ |
| | TTA TG-3′ | | |
| Reverse | 5′-GGA TCA AAC GGC | 5′-ATG CTT AAG TTC | 5′-GGT CGG CAC GAA |
| primer | CTG CAC A-3′ | AGC GGG TAG-3′ | TTT CAT ATA AG-3′ |
| TAQMAN ® | | 5′-FAM-6CTG GGT TTG | |
| probe | | GTG TTG AGC AAT | |
| | | ACG-BHQ1-3′ | |
| Amplicon | 278 | 211 | 89 |
| size | | | |
| (bp) | | | |

A six-parameter sigmoid equation with parameters determined by a Levenberg-Marquardt (LM) regression process was first obtained to provide the best-fit for a PCR dataset The threshold cycle number (Ct) was then calculated from select parameters of the fit. A modified sigmoid model was used in the fit to account for non-ideal issues such as baseline drift, inefficient amplification, and lack of saturation phase, providing a quality fit for most practical quantitative PCR conditions.

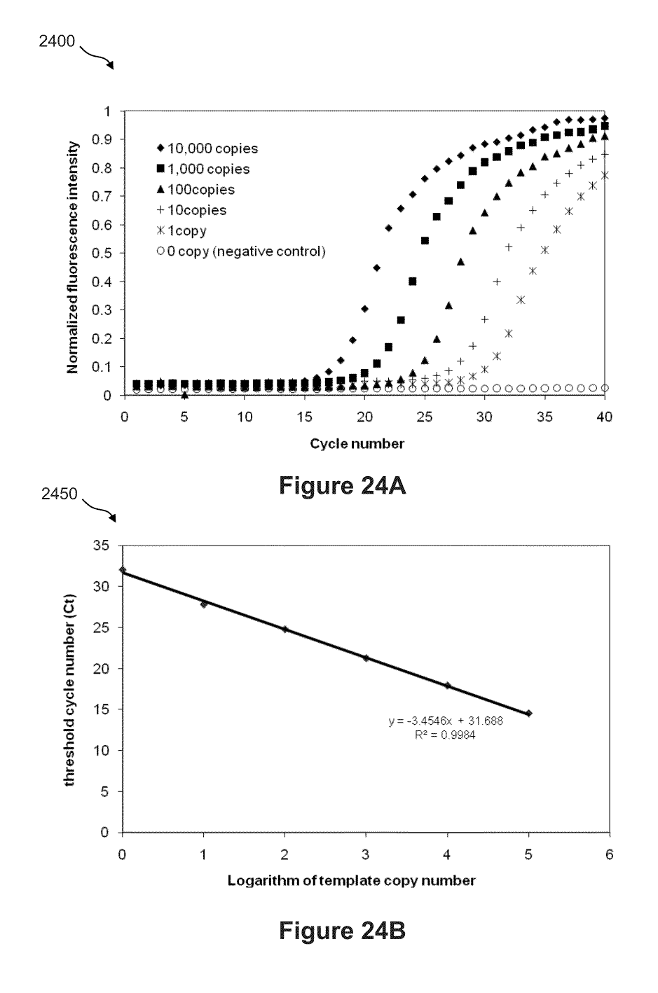

FIGS. 24A and 24B show an example of a plot 2400 of real-time PCR data and a plot 2450 of PCR efficiency, respectively, of real-time PCR detection of methicillin resistant Staphylococcus aureus (MRSA) genomic DNA. A titration experiment was performed using 10-fold dilutions of DNA samples that ranged from 307 picograms to 3.07 femtograms of input DNA, which is about the equivalent of 10,000 to 1 MRSA genome copies. The thermocycling conditions were 60 seconds (s) hot-start at 95° C., followed by 40 cycles of 10 sec denaturation at 95° C. and 30 sec annealing/extension at 60° C. The experiment was repeated 3 times on different droplet actuators. FIG. 24A shows real-time PCR data for DNA inputs of 1 to 10,000 genomic equivalents and the negative control. The average threshold cycle numbers and standard deviations were 14.52±0.15, 17.90±0.11, 21.24±0.18, 24.78±0.24, 27.81±0.12, and 32.05±1.67 for 105, 104, 103, 102, 101, and 100 genome equivalents, respectively.

Following real-time PCR, PCR droplets were collected from the droplet actuator and analyzed by gel electrophoresis (results not shown). The amplified products were of the expected length and no by-products were observed. The PCR amplification was highly reproducible as indicated by the small standard deviations of the Ct values. The larger standard deviation for the single-copy experiments is most likely due to the sampling variability at such low copy numbers. Samples containing a single genome equivalent were reliably amplified during the experiments, confirming that sensitivity was adequate for the detection of a single organism.

FIG. 24B shows the linear regression of the average Ct values versus the logarithm of the amount of input DNA. The slope extracted from the linear fit may be used to calculate the reaction efficiency based on the following equation²⁹⁵.

efficiency=10^{-(1/slope)}−1

The calculated amplification efficiency of the PCR system was 94.7%, which is in the range of conventional bench-top thermocycles and is superior to most miniaturized flow-through PCR devices²⁹⁵.

The digital microfluidic PCR system exhibited excellent limit of detection, reaction efficiency, and reproducibility, which may be attributed to a combination of factors. First, in the oil environment a thin film of filler fluid exists between the droplets and droplet actuator surfaces, which minimizes the direct droplet-substrate contact. Second, the closed-loop format results in a moderate surface-to-volume ratio which is a fraction (1/cycle number) of the value for a traditional flow-through configuration. Finally, additives such as surfactants and supplemental amount of reagents in the PCR formulation can passivate the surface and stabilize the reaction components in the droplets. All of these factors effectively minimize surface-induced reaction inhibition and provide improved PCR performance.

7.3.8.3 Thermal Cycling Speed and Variable Cycle-Time Thermal Cycling

Ultra rapid PCR has been a goal for many of the miniaturized PCR devices. Although miniaturization enables faster thermal cycling, incubation times (dwell times) may become limited by reaction kinetics with further reductions in cycle time available only at the expense of reaction yield. To optimize PCR speed and amplification efficiency, the kinetics of amplicon production at different stages of a 40-cycle PCR was analyzed.

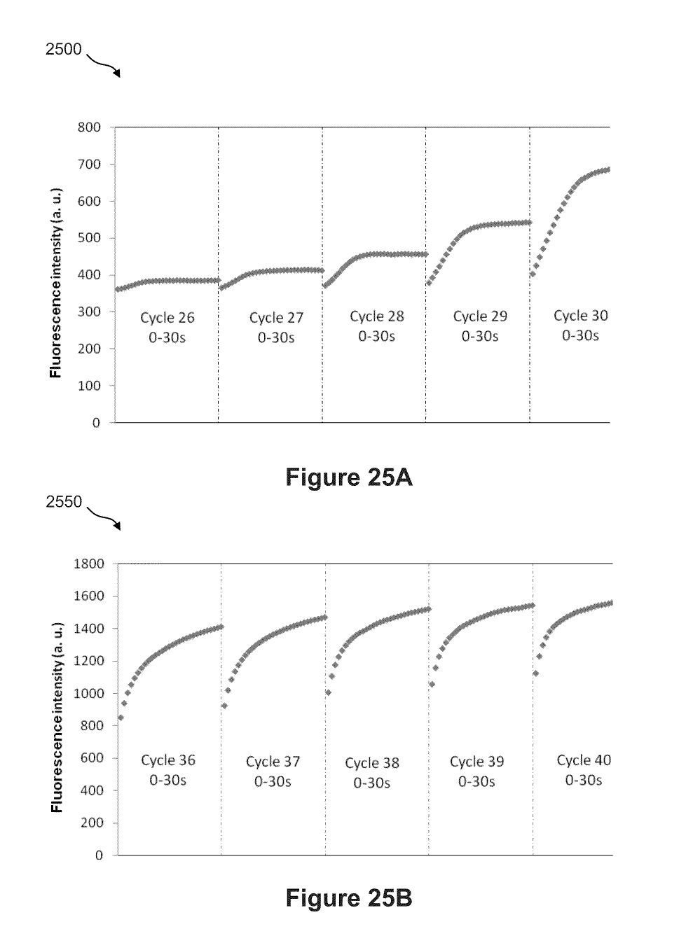

FIGS. 25A and 25B show plots 2500 and 2550, respectively, of fluorescence intensity data for cycles 26 through 30 and cycles 36 through 40, respectively, for a 40 cycle real-time PCR. A method described by Neuzil et al³⁰ was used to monitor the increase in fluorescence within each annealing and extension cycle. For a 10¹ dilution of MRSA DNA (30.7 fg) with 10 sec incubation at 95° C. and 30 sec incubation at 60° C. the amplicon was detected at a Ct of 27.8. The fluorescence signal increase within cycles 26 through 30 and cycles 36 through 40 were measured. FIG. 25A shows fluorescence intensity data for the exponential amplification phase of the reaction. FIG. 25B shows fluorescence intensity data for the saturation phase of the reaction. For every cycle, there was an initial increase in the fluorescence signal followed by a plateau, from which the actual time required to complete the extension of all amplicons in a particular cycle may be estimated. The data show that the completion time increased with the cycle number at the beginning of the exponential phase and then decreased when entering the saturation phase. Before the threshold cycle, 10 sec appears sufficient to achieve full amplification for each cycle, likely due to the fact that the DNA template is present in small quantities compared to the excess reaction components enabling the reaction to proceed quickly.

The PCR reaction was repeated with varying annealing extension cycle times corresponding to the values required for reaction completion estimated from FIG. 25, which were 10 sec for cycles 1 through 25, 30 sec fix cycles 26 through 35, and 20 sec for cycles 36 through 40. The denaturation time was fixed at 6 sec for all 40 cycles. The total annealing/extension time for this variable cycle time protocol was 650 sec. A control experiment was also performed in which the same 650 sec total annealing/extension time was achieved by dividing the total time evenly between all 40 cycles resulting in a fixed cycle time of 16 sec.

FIG. 26 shows an example of a plot 2600 of fluorescence intensity data for a comparison of real-time PCR using fixed (2 reactions) and variable cycle (1 reaction) times. Other conditions (e.g., temperatures, volumes, template, reagents) were identical for the three reactions. The input template was 30.7 fg MRSA genomic DNA (equivalent to 10 MRSA genome copies). The two fixed cycle time protocols consisted of 10 sec (6 sec) denaturation at 95° C. and 30 sec (16 sec) annealing/extension at 60° C. throughout 40 cycles. The variable cycle time protocol consisted of 6 sec denaturation at 95° C. throughout 40 cycles, and annealing/extension of 10 sec for cycles 1 through 25, 30 sec for cycles 26 through 35 and 20 sec for cycles 36 through 40 at 60° C. Based on a comparison of the cycle threshold and reaction yield, PCR performance with varying cycle times was equivalent to the original 30 sec fixed cycle time protocol, but the total annealing/extension time was reduced 46%. The control experiment with the same total reaction time achieved with a 16 sec fixed cycle time had significantly reduced reaction yield, demonstrating the benefit of the variable cycle time protocol for faster PCR reactions. With 6 sec for denaturation and 4 sec for droplet transport included in each variable cycle, the total time required to complete a 40 cycle PCR of MRSA genomic DNA with optimal reaction yield was 28 min. This result was obtained using platinum Taq polymerase (Invitrogen, CA) and may be improved through the use of faster polymerases which are commercially available.

The results demonstrate the feasibility of using digital microfluidics to optimize real-time PCR by using variable cycle times to adjust reaction conditions without sacrificing reaction efficiency. In one example, PCR amplification of an unknown sample may be managed using a “smart” software program which continuously monitors the fluorescence signal increase within each cycle and automatically triggers the next cycle once the signal reaches a plateau. Digital microfluidics is well-suited for this type of optimization because incubation times and many other parameters may be dynamically recalibrated in real-time which are impossible with many other less flexible formats.

7.3.8.4 PCR Multiplexing

In one embodiment, the digital microfluidic PCR droplet actuator may be configured to perform multiplexed PCR analysis by separating reactions in space rather than by spectral multiplexing. In one example, a DNA sample may be loaded onto the droplet actuator and subsequently divided using droplet operations into a set of sub-sample droplets. Each sub-sample droplet may be combined with a reagent droplet that contains specific primer sets and/or probes in addition to PCR master mix to yield a specific PCR reaction droplet. The number of sub-samples that may be generated from a single starting DNA sample is virtually unlimited but the sensitivity of the PCR assay may be reduced each time the sample is sub-divided. In this example, all of the PCR reaction droplets are circulated within a single common thermocycling loop and sequentially passed through the same detection spot allowing analysis of multiple genetic targets in a single DNA sample. The four individual thermocycling loops (referring to FIG. 22) may be used to achieve multi-target PCR analysis of up to four different DNA samples in parallel, thereby adding another level of multiplexing.

In another example, fluorescent reporters with multiple different wavelengths may also be employed in each PCR reaction droplet to further increase multiplexing. In this example, a four channel fluorimeter with multiple wavelength detectors would be used.

A multichannel two-plex PCR assay was conducted with the assay configuration shown in Table 8.

TABLE 8
Configuration and results of a parallel 2-plex PCR assay

| | | | PCR results | |
|---|---|---|---|---|
| | DNA sample composition | | | Droplet #2 |
| | MRSA | Mycoplasma | Droplet #1 | Mycoplasma |
| | DNA | DNA | MRSA primer | primer |
| Loop 1 | 1.6 pg/μl | — | Positive | Negative |
| (sample A) | | | | |
| Loop 2 | — | 1.6 pg/μl | Negative | Positive |
| (sample B) | | | | |
| Loop 3 | 1.6 pg/μl | 1.6 pg/μl | positive | Positive |
| (sample C) | | | | |

The PCR protocol was programmed to first distribute a 1× (i.e., 330 nL) droplet of PCR mix containing MRSA primers to each loop from a common reagent reservoir, followed by a 1× droplet of PCR mix containing Mycoplasma primers. Two 1× sample droplets were dispensed from each DNA sample reservoir (i.e., MRSA DNA sample and Mycoplasma DNA sample), transported to the designated loop and combined using droplet operations with each of the two previously distributed PCR mix droplets containing MRSA or Mycoplasma primers. The two-plex real-time PCR was then conducted by circulating the two 2× droplets in each loop and passing each droplet through the detection spot once per cycle.

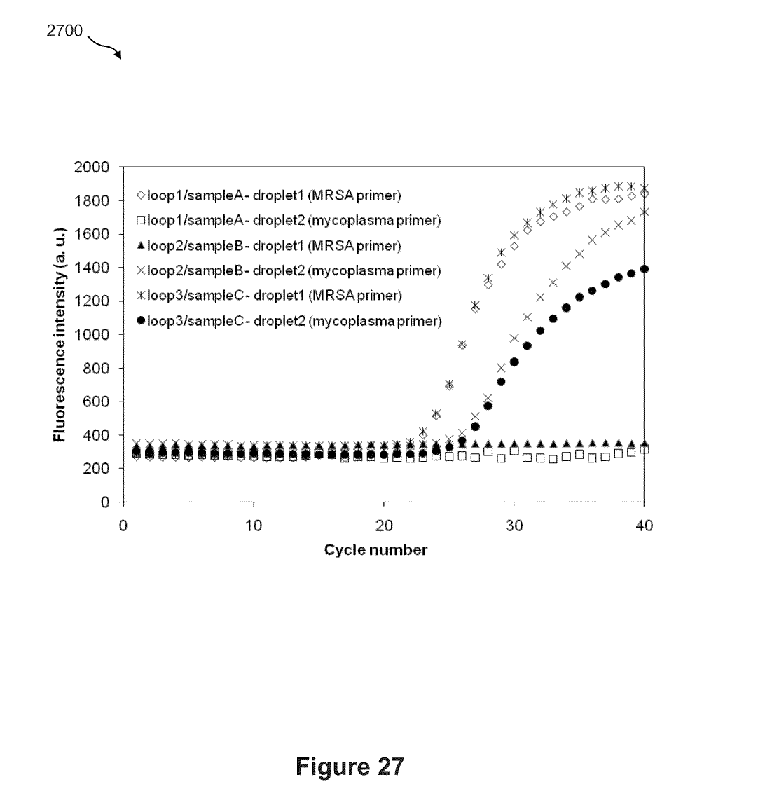

FIG. 27 shows an example of a plot 2700 of fluorescence intensity data of a two-plea (MRSA and Mycoplasma) real-time PCR assay of Table 5 performed in parallel on the digital microfluidic PCR platform. All of the DNA targets in the three samples were successfully detected with no false positive results and the threshold cycles were comparable with the single-plex PCR.

A common problem for microchip PCR is the non-specific adsorption of DNA molecules to the droplet actuator surface, which may be mitigated by various means but remains difficult to completely eliminate. Loss of DNA to droplet actuator surfaces may result in reduced sensitivity as well as cross-contamination between samples transported through shared pathways. Even one DNA template inadvertently transferred between samples may be exponentially amplified and result in a false positive result. This is particularly likely to occur if different samples are amplified serially because even the carry-over of 1 part per billion from a completed reaction may contaminate the subsequent reaction. For this reason, the digital microfluidic PCR droplet actuators are designed to be used once and discarded which is made economically feasible by their low cost PCB construction. Additionally, the droplet actuators are designed so that the pathways of droplets from different samples never intersect.

In the multiplex PCR experiment described in Table 5 and FIG. 27, all of the droplets that were circulated in a common loop originated from the same DNA sample so cross-contamination of the samples was not a concern. The possibility of carry-over of primers, amplicons or reporters between reactions still exists but these contaminants are not exponentially amplified in the reaction. As confirmed by the negative controls in the two-plex experiment (negative droplet #2 in loop 1 and negative droplet #1 in loop 2) this form of contamination may not be significant. In these experiments different DNA samples were simultaneously amplified in separate loops located within a common reservoir of oil. Although the filler oil may potentially provide another route for cross-contamination this problem was not observed. The data demonstrates the feasibility of combined spatial and time-division multiplexing which may significantly increase the assay throughput of the digital microfluidic PCR system.

7.3.8.5 Selection of Filler Fluid

A suitable filler fluid for electrowetting-based PCR assays should be inert, thermally stable, and have a viscosity low enough to easily facilitate droplet transport. In one example, hexadecane is a suitable filler fluid based on these criteria and may be used with PCR protocols that use a fluorescence detection protocol such as incorporation of EVAGREEN® dye (available from Biotium, Inc., Hayward, Calif.). Thus, in one example, one or more hydrocarbon oils are used as a filler fluid during electrowetting mediated thermal cycling in a PCR reaction that utilizes EVAGREEN® dye. For example, the hydrocarbon oils may be alkane hydrocarbon oils. For example, the hydrocarbon oils may have 10, 11, 12, 13, 14, 15, 16, 17, 18, 19, or 20 carbons. Similarly, in a related example, the alkane hydrocarbon oils may have 10, 11, 12, 13, 14, 15, 16, 17, 18, 19, or 20 carbons. In another example, 2 cSt silicone oil may be used as a filler fluid with PCR protocols (e.g., quantitative PCR) that use TAQMAN® probes as a fluorescence detection method.

Bubble formation in the filler fluid is a potentially important concern when PCR in performed in an enclosed microfluidic device. Formation of bubbles during PCR may be substantially eliminated by degassing hexadecane for 2 hr before filling the PCR droplet actuator. Silicone oil requires longer a degassing treatment due to its greater gas solubility and in some cases bubble formation may not be completely eliminated. However, compared to channel-based and pressurized PCR microchip devices, the digital microfluidic PCR platform is substantially less vulnerable to bubbles because there are no fixed channels that may become blocked by bubble formation. Even when small bubbles are formed directly in the transport path of a droplet they typically do not interfere with the reaction because the droplets are able to displace the bubble into the surrounding oil phase.

7.3.8.6 Manipulation of Magnetically Responsive Beads

Magnetically responsive beads are commonly used in biological and clinical assays to capture or immobilize targets of interest such as DNA, whole cells or specific antigens. Magnetically responsive beads provide a convenient means for concentrating these targets of interest and transferring them between different liquid mediums.

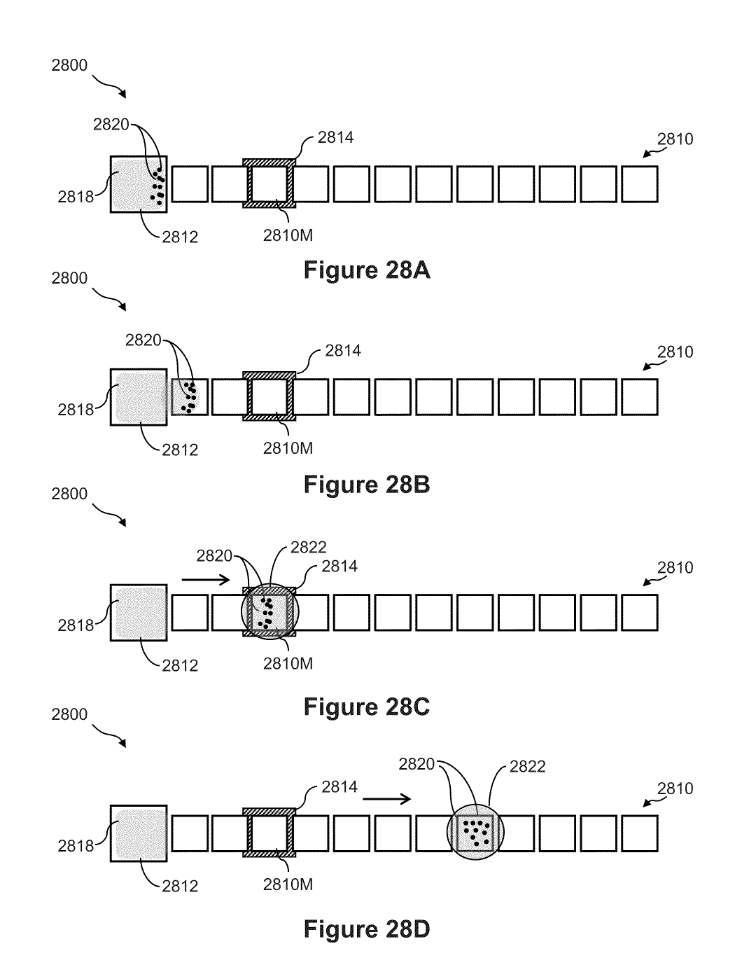

FIGS. 28A through 28D illustrate top views of an example of a portion of an electrode arrangement 2800 of a droplet actuator (not shown) and show a process of concentrating and dispensing magnetically responsive beads onto a droplet actuator. In one embodiment, the method of the invention of FIGS. 28A through 28D may be used to load a larger sample volume (e.g., 5 μl to 10 μL conventional sample size) onto a droplet actuator for subsequent processing and analysis in smaller microfluidic volumes (e.g., 330 nL) without compromising assay sensitivity.

Electrode arrangement 2800 may include an arrangement of droplet operations electrodes 2810 (e.g., electrowetting electrodes) and a dispensing electrode 2812 that is configured for dispensing a volume of fluid. Droplet operations are conducted atop droplet operations electrodes 2810 on a droplet operations surface. A magnet 2814 is arranged in close proximity to droplet operations electrodes 2810. In particular, magnet 2814 is arranged such that certain droplet operations electrodes 2810 (e.g., droplet operations electrode 2810M) are within the magnetic field thereof. Magnet 2814 may, for example, be a permanent magnet. A volume of fluid 2818 that includes a quantity of magnetically responsive beads 2820 (e.g., 5 ng/μl) may be present at dispensing electrode 2812. Fluid 2818 may, for example, be a sample fluid in which a target of interest (e.g., nucleic acid, cells, or a specific antigen) is bound to magnetically responsive beads 2820. Fluid 2818 may, for example, have a volume of about 5 μL to about 10 μL. Thus, in one embodiment, the invention provides a droplet actuator with electrodes for conducting droplet operations, including an electrode that is positioned in proximity to a sensor of sensing a signal from the droplet and a magnet positioned such that when a droplet is positioned at this electrode, any magnetically responsive beads are pulled aside, such that the sensing of the signal by the sensor can be effected without substantial interference from the magnetically responsive beads.

An example of a process of concentrating and dispensing magnetically responsive beads onto a droplet actuator may include, but is not limited to, the following steps.

In one step. FIG. 28A shows fluid 2818 with magnetically responsive beads 2820 therein positioned at dispensing electrode 2812. Because of the magnetic field of magnet 2814, magnetically responsive heads 2820 are aggregated at the edge of fluid 2818.

In another step, FIG. 28B shows a finger of fluid from dispensing electrode 2812 is pulled using droplet operations along adjacent droplet operations electrodes 2810. Because of the magnetic field of magnet 2814, magnetically responsive beads 2820 remain at the front edge of the finger of fluid.

In another step. FIG. 28C shows a 1× droplet 2822 (i.e., 330 nL droplet) is dispensed using droplet operations from dispensing electrode 2812. Droplet 2822 includes substantially all of magnetically responsive beads 2820 from fluid 2818. The strength of the magnetic field of magnet 2814 is sufficiently strong enough to concentrate the magnetically responsive beads 2820 within the droplet 2822 and permit liquid exchange, but not strong enough to pull the beads through the oil-water interface at the droplet's meniscus. Therefore, when the droplet is transported out of the magnetic field of magnet 2814, the beads are retained inside the droplet.

In another step, FIG. 28D shows droplet 2822 transported using droplet operations out of the magnetic field of magnet 2814. As droplet 2822 is transported out of the magnetic field of magnet 2814, the intrinsic fluid circulation within a moving droplet quickly resuspends and disperses magnetically responsive beads 2820 within droplet 2822. In this example, magnetically responsive beads 2820 from a fluid volume of 5 μL to 10 μL have been concentrated into a single 330 nL droplet.

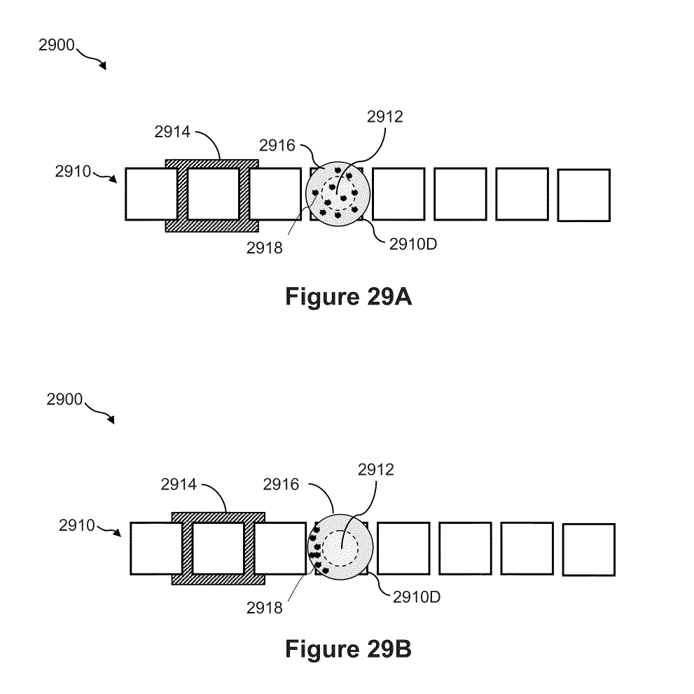

FIGS. 29A and 29B illustrate top views of an example of a portion of electrode arrangement 2900 of a droplet actuator (not shown) and show a method of manipulating magnetically responsive beads to improve analyte detection. Electrode arrangement 2900 may include an arrangement of droplet operations electrodes 2910 (e.g., electrowetting electrodes). Droplet operations are conducted atop droplet operations electrodes 2910 on a droplet operations surface. A detection spot 2912 is provided at a certain droplet operations electrode 2910D. A magnet 2914 is arranged in close proximity to droplet operations electrodes 2910. In particular, magnet 2914 is arranged such that certain droplet operations electrodes 2910 (e.g., droplet operations electrode 2910M) are within the magnetic field thereof. Magnet 2914 may, for example, be a permanent magnet or an electromagnet. Magnet 2914 is positioned in proximity of detection spot 2912.

An example of a method of manipulating magnetically responsive beads to improve analyte detection on a droplet actuator may include, but is not limited to, the following steps.

In one step, FIG. 29A shows a sample droplet 2916 that has magnetically responsive beads 2918 therein positioned at droplet operations electrode 291011), which is within the range of detection spot 2912. Detection spot 2912 is typically smaller in diameter than sample droplet 2916. Magnetically responsive beads 2918 are dispersed within sample droplet 2916 including the area occupied by detection spot 2912 and may interfere with detection of the amplification signal, e.g., fluorescent dyes by blocking the fluorescent light from the detection device (i.e., a fluorimeter), resulting in scattered readings and background noise.

In another step, FIG. 29B shows magnetically responsive beads 2918 aggregated at the edge of sample droplet 2916. Magnet 2914 is positioned at a sufficient distance from droplet operations electrode 2910D and sample droplet 2916 to provide a sufficient magnetic field to gently attract and aggregate magnetically responsive beads 2918 to the edge of sample droplet 2916 and away from detection spot 2912. The strength of the magnetic field provided by magnet 2914 is such that magnetically responsive beads 2918 do not form a tight aggregate and may be easily redistributed in subsequent droplet operations.

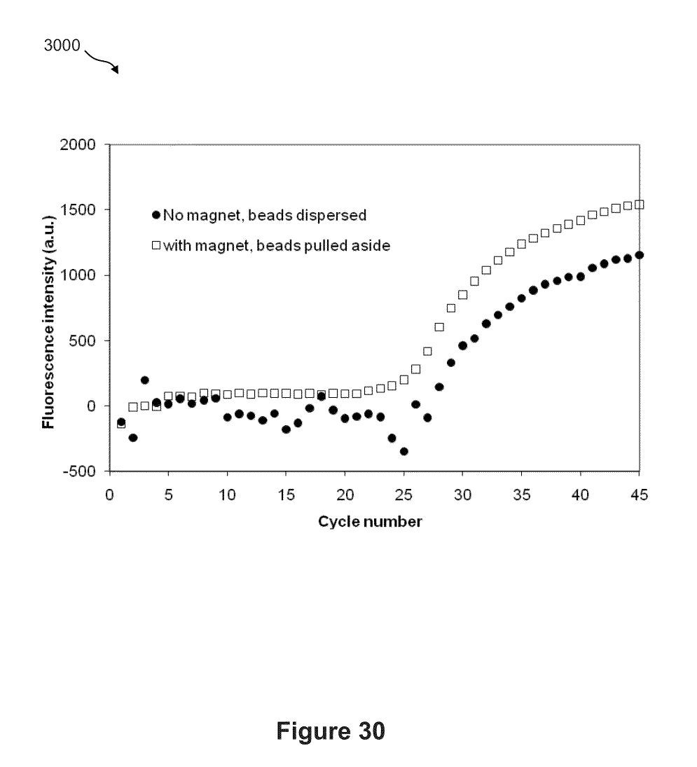

FIG. 30 shows an example of a plot 3000 of fluorescence intensity data for real-time PCR performed with and without an external magnet positioned in proximity to the detection spot. PCR reactions were performed with 2.5 μg magnetically responsive DYNABEADS® beads added to a 660 nL reaction droplet.

7.3.8.7 Pathogen Detection

Digital microfluidic technology that combines sample concentration and solution exchange (e.g., washing and/or elution) using magnetically responsive beads on a droplet actuator may be used to detect pathogens that are normally present in low concentrations in real biological specimens.

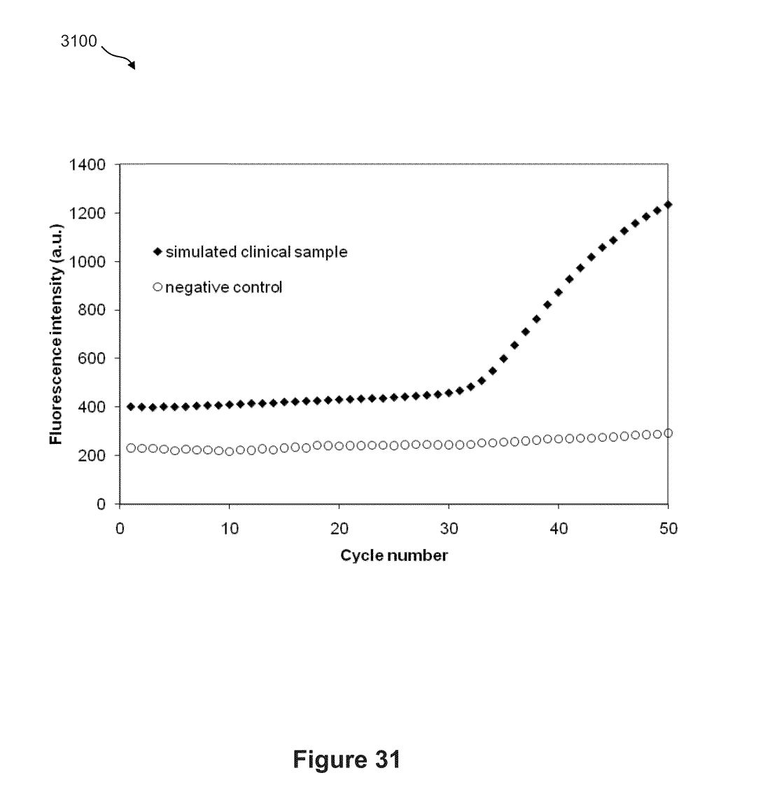

FIG. 31 shows an example of a plot 3100 of fluorescence intensity data from a real-time PCR analysis of Candida athicans DNA in a simulated clinical sample. Human whole blood was spiked with C. albicans yeast cells, and processed on-bench with a bead-based DNA extraction and purification protocol. The microbial DNA was captured on magnetically responsive DYNABEADS® heads using an oligonucleotide capture probe and suspended in 6 μL TE buffer. A negative control was prepared from whole blood without addition of C. albicans. The 6 μL head-containing sample was loaded into a dispensing reservoir on the droplet actuator. A 330 nL droplet that contains substantially all of the beads was dispensed and merged with a 330 nL droplet of PCR reaction mix. The combined droplet was then thermocycled with 120 sec hot-start at 95° C. Mowed by 50 cycles of 10 sec at 95° C. and 45 sec at 58° C. Negative controls were processed using the same protocol. Target-specific TAQMAN® probes were used for better specificity and consequently 2 cSt silicone oil was used as the filler fluid. C. albicans DNA from the simulated clinical sample was successfully amplified.

7.3.9 Detection of Mycoplasma pneumoniae

In recent years, real-time PCR has emerged as a rapid and sensitive diagnostic technique for respiratory infections caused by M. pneumoniae (e.g., community acquired pneumonia (CAP)), an organism whose detection by culture and serology has long been recognized as arduous and ineffectual. Consequently, many variations on DNA-based detection of M. pneumoniae in clinical specimens have been developed, and most have demonstrated significantly greater sensitivity and specificity than culture or serology^{31, 32}. These conventional PCR methods have not become routine in hospital clinical laboratories or outpatient clinics because they require significant training and most of the protocols and instruments are not expandable to multiple pathogens, fully automated or portable. The digital microfluidic PCR platform of the invention obviates many of the disadvantages of conventional PCR thermocycling.

Digital microfluidic real-time PCR and conventional real-time PCR were compared for detection of M. pneumoniae DNA in respiratory specimens from patients with CAP using the same protocol for extraction of DNA and probe and primers for PCR.

7.3.9.1 Extraction of M. pneumoniae DNA

Genomic DNA was prepared from a reference strain of M. pneumoniae (ATCC #15531) and nasopharyngeal wash (NPW) specimens from healthy human volunteers (control NPW) and clinical subjects. NPWs were collected by using a sterile syringe to spray at least 2 mL sterile saline (0.9% NaCl) into one nostril and asking the subject to expel the saline into a sterile tube. All control NPW specimens were stored at 4° C. for future tests, while clinical NPW specimens were stored at −80° C. Simulated NPW were prepared by seeding sterile saline with dilutions of a stock culture of M. pneumoniae to create 10-fold concentrations of CFU/mL. M. pneumoniae was also seed into NPWs from healthy volunteers.

Genomic DNA was prepared using an on-bench extraction protocol using magnetically responsive beads. The reagents and conditions in the extraction protocol are compatible with both conventional and digital microfluidic PCR assays.

DNA extraction was carried out as follows: 200 μL of each real or simulated NPW specimen was treated directly with 20 μL proteinase K and 200 μL AL buffer (both available from QIAGEN Inc., Valencia, Calif.). These specimens were vortexed for 5 sec, briefly centrifuged to clear caps, and incubated at 56° C. for 15 minutes. Then, 4.5 μL oligonucleotide capture probe (5′-Biotin-AGAGTGGATCTTCTGACACTTCCGGGTCTAAC-3′, Sigma-Aldrich Co.) was added to each specimen, and the mixture was vortexed for 5 sec, briefly centrifuged, and incubated at 95° C. for 15 min and 56° C. for 20 min to denature the target DNA and allow the probe to hybridize. Pre-washed m-270 Streptavidin DYNABEADS® beads (5.0 μL) were added to each tube. Tubes were incubated at room temperature for 30 minutes, during which time they were mixed by inversion 5 times every 5 minutes. The specimens were then centrifuged for 5 sec and placed on the magnetized rack for 10 minutes, after which the liquid was withdrawn from each specimen, leaving the beads with bound target DNA. The beads were washed with 200 μL TE buffer, gently mixed and returned to the magnetized rack for 2 minutes. The beads were washed once more and gently resuspended in 10 μL TE buffer with 50 mM NaCl and 0.075% (w/v) TWEEN® 20 Magnetic beads with bound DNA were used immediately or stored at −80° C. until analysis. Preliminary experiments confirmed that DNA was not degraded by storage. For real-time PCR analysis, each 10 μL bead-DNA preparation was split: 6 μL were used for conventional real-time PCR and 4 μL for the digital microfluidic platform.

7.3.9.2 Conventional Real-Time PCR

M. pneumoniae-specific primer pairs were selected to amplify sequences of the single copy P1 adhesion gene³³ and the multicopy genes, MP5³⁴, repMP1³⁵, and Mp181³⁶. The Mp181 primers amplify specific sequences of the mycoplasmal community-acquired respiratory distress syndrome (CARDS) toxin gene sequences, which proved to be the most sensitive and generated an optimal 73-bp amplicon. A thorough search of GenBank sequences found no variation among strains at the location of either the capture probe or the PCR probe and primers.

Conventional real-time PCR was performed using an Applied Biosystems real-time PCR system, available from Applied Biosystems, Inc. (Foster City, Calif.). Each real-time PCR assay plate included a positive control of simulated NPW containing M. pneumoniae at 10⁴ CFU/mL and two non-template controls (NTC). One NTC consisted of sterile saline that had been concurrently subjected to the extraction protocol, and a second NTC consisted of PCR grade water used in place of extracted specimen template. All controls and simulated or real NPW specimens were processed in parallel from DNA extraction to assay, and all were assayed in triplicate.

The Mp181 primer sequences and probe were as follows³⁶: Mp181-F 5′-TTTGGTAGCTGGTTACGGGAAT-3′; Mp181-R 5″-GGTCGGCACGAATTTCATATAAG-3′; Mp181 [FAM]-TGTACCAGAGCACCCCAGAAGGGCT-[BHQ]). Each PCR well had a final volume of 20 μL and contained 250 nM of each primer, 10 μL TAQMAN® Fast Universal Real-Time Master Mix, 0.58 U uracil-DNA glycosylase (UNG, Applied Biosystems), and 2.0 μL DNA-bead extract. The master mix included UNG (from New England Biolabs, Inc., Ipswich, Mass.) and UTP to prevent amplification of any previously made amplicons. The following program was used with the thermocycler (7900 HT, Applied Biosystems): Initial amplification at 95° C. for 120 sec, followed by 45 cycles each at 95° C. for 15 sec and 57.8° C., for 30 s, and concluding with a final cycle at 95° C. for 60 sec and 55° C. for 60 sec.

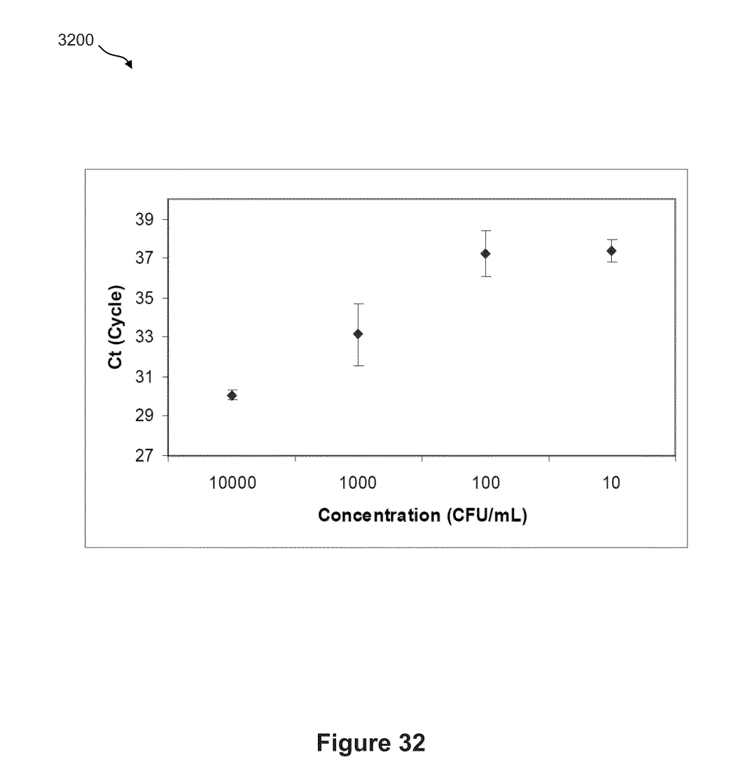

FIG. 32 shows an example of a plot 3200 of M. pneumoniae concentration versus mean Ct of simulated clinical samples assayed on a conventional real-time PCR platform. Evaluation of magnetic bead DNA extraction and real-time PCR precision was assessed by repeated extractions and multiple reactions of simulated clinical samples. These samples were created by serial dilution of the M. pneumoniae reference culture to 10⁴, 10³, 10², and 10¹ CFU/mL saline. Two sets of samples underwent magnetic bead DNA extraction on consecutive days, and samples were reacted in quadruplicate and triplicate, respectively. These data also served to determine assay sensitivity.

### 7.3.9.3 Digital Microfluidic Real-Time PCR

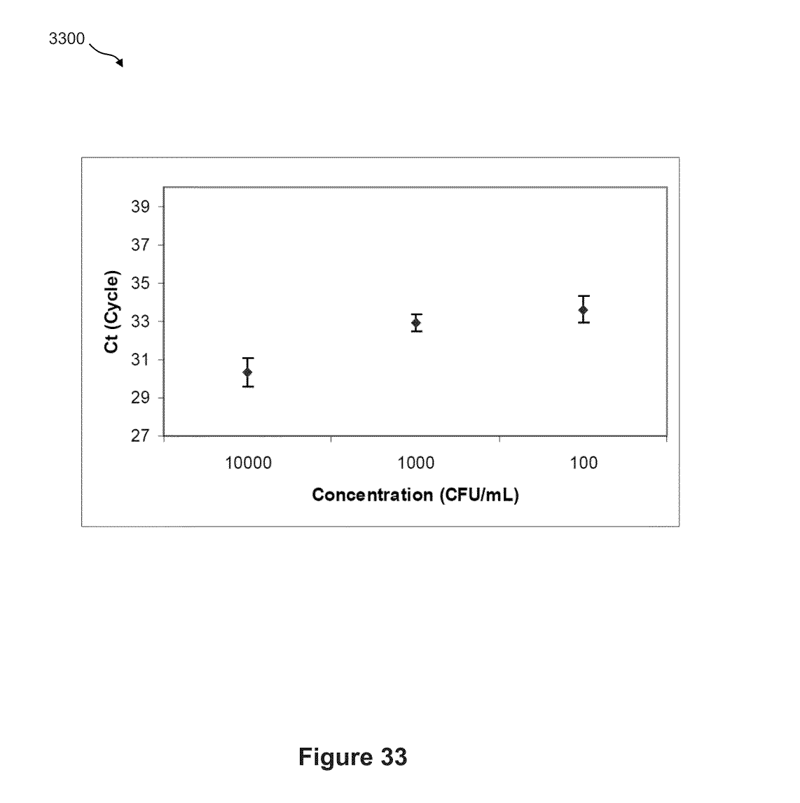

FIG. 33 shows an example of a plot 3300 of M. pneumoniae concentration versus mean Ct of simulated clinical samples assayed on the microfluidic real-time PCR platform. The precision of microfluidic real-time PCR was validated using five simulated clinical samples. The samples were prepared at a concentration of 10⁴ CFU/mL in sterile saline and extracted as described above using magnetically responsive beads. The final 10 μL of each sample were pooled. Twenty real-time PCR reactions were run on the digital microfluidic platform, consisting of four reactions each on five separate, four-loop droplet actuators (referring to FIG. 22). A 10-fold dilution series of M. pneumoniae was used to generate a standard curve comparing CFU/mL with the real-time PCR cycle threshold of detection. Three real-time PCRs of each concentration were run on the digital microfluidic platform, and each run included a NTC consisting of PCR grade water in place of extracted specimen template. In addition, separate studies using beads added to spin column-purified M. pneumoniae DNA were performed to demonstrate that magnetically responsive beads on the droplet actuator did not inhibit the PCR (data not shown).

Two 2-μL aliquots of each NPW extract were analyzed. Each 2 μL extract was diluted 1:1 in TE buffer (pH 8.0, 50 mM NaCl, 0.075% TWEEN® 20), which was necessary because the droplet actuator dispensing reservoirs were designed to accept a minimum input volume of 3 μL. Digital microfluidic real-time PCR was performed on the two NPW DNA samples as close to the same time as possible and always on separate droplet actuators. Three patient sample aliquots and one positive control, consisting of 200 fg/μL of M. pneumoniae genomic DNA, were run on each droplet actuator. Because previous tests of NTCs were invariably negative and to maximize throughput, negative controls were included only periodically. The PCR master mix was prepared daily and included, per reaction, 1.2 μL PCR buffer with final concentrations after 1:1 mixing of sample and master mix droplets of 3 mM MgCl₂, 1 Mp181 primers, 1 μM TAQMAN® Mp181 probe, and 0.5 U/μL KAPATaq. For on-chip real-time PCR, the temperature control elements were preheated to 95° C. and 58° C. The droplet actuator was then removed from its vacuum-sealed package, filled with about 1.5 mL degassed 10 cSt silicone oil, and inserted into the real-time PCR instrument.

Four microliters of magnetic bead-extracted DNA were added to the appropriate sample dispensing reservoirs, and 3 μL of PCR master mix were added to the appropriate reagent dispensing reservoirs. The magnetically responsive DYNABEADS® beads are concentrated by permanent magnets embedded in the deck of the instrument and position in proximity to certain droplet operations electrodes on the droplet actuator when in place on the real-time instrument. A 330 nL droplet containing all the beads concentrated from the 4 μL sample was dispensed from the loading zone and mixed with a 330 nL droplet of PCR master mix. The combined droplet was physically cycled using droplet operations between the denaturation and annealing zones corresponding to the cycle temperatures of 95° C. and 58° C., respectively. The dwell times (incubation time) were 10 sec at 95° C. and 45 sec at 58° C. with 4 sec of transport time between the two zones. Fluorescence was determined at the end of each annealing extension cycle. At the conclusion of the run, the software program computed the earliest cycle threshold (Ct) of detection of amplicon above the baseline, that is, a positive test for the target DNA. The Ct is inversely related to the amount of template DNA. Patient specimens that were discrepant between the two platforms were re-extracted and re-tested on both platforms.

7.3.9.4 Validation of Conventional and Digital Microfluidic PCR

Positive control samples for conventional real-time PCR were created by extracting a 10⁴ CFU/mL suspension of M. pneumoniae in sterile normal saline. All 29 conventional real-time PCR positive control samples tested alongside patient samples amplified with a mean cycle threshold (Ct) of 29.7+/−0.50 SD. The efficiency of conventional real-time PCR runs varied from 91.6 to 101.3%. Alt but one. NTC tested by conventional real-time PCR were negative. After finding amplification of one NTC sample on the conventional real-time PCR, all reagents were replaced, and the tests on that run were successfully repeated.

Nineteen of the 20 aliquots of 10⁴ CFU/mL analyzed by real-time PCR on the microfluidic platform amplified. The mean Ct+/−SD for these runs was 29.6+/−0.96. The results are shown in Table 9. Positive controls for the microfluidic real-time PCR platform consisted of 67 fg commercial M. pneumoniae DNA. Thirty-seven of forty positive controls were amplified with a mean Ct of 31.28+/−0.98 SD. The Us for these controls was comparable to the results obtained by Wirtchell et al³⁶.

TABLE 9
Precision of the microfluidic real-time PCR platform
using simulated clinical samples at 10⁴ CFU/mL

| Droplet | | | | | |
|---|---|---|---|---|---|
| Actuator | Loop 1 | Loop 2 | Loop 3 | Loop4 | Mean ± SD |
| 1 | 31.1 | 30.3 | 30.1 | 29.5 | 30.25 ± 0.61 |
| 2 | 29.7 | 29 | 28.5 | 29.7 | 29.24 ± 0.59 |
| 3 | 29.4 | 28.6 | 29.6 | 28.9 | 29.14 ± 0.45 |
| 4 | 29.1 | 28.2 | 28.9 | 28.4 | 28.68 ± 0.42 |
| 5 | 30 | 31 | 31.7 | ND | 30.94 ± 0.85 |
| Mean | 29.86 ± | 29.42 ± | 29.76 ± | 29.13 ± | 29.58 ± 0.96 |
| | 0.69 | 1.06 | 1.12 | 0.51 | |

SD, Standard Deviation; ND, not done

7.3.9.5 Comparison of Conventional and Digital Microfluidic PCR

The limit of detection of M. pneumoniae in simulated specimens extracted using magnetically responsive beads was 10 CFU/mL for the conventional real-time PCR platform. The microfluidic platform was not tested at lower concentrations but did detect 100 CFU/mL. The limit of detection was more sensitive on both platforms for specimens extracted via QIAamp DNA blood mini kit (data not shown), which is consistent with a study comparing five DNA extraction methods, including the QIAamp DNA mini kit and magnetic bead extraction with Dynabeads³⁷. That study showed the QIAamp mini kit produced a higher yield of extracted DNA than did magnetic bead extraction, which should lead to increased sensitivity.

Six of the 59 patient NPWs initially were positive for M. pneumoniae DNA on at least one real-time PCR platform. Two NPWs tested positive for M. pneumoniae DNA on both the conventional and the digital microfluidic platforms. Two NPWs were positive only on the conventional platform, and two others were positive only on the digital microfluidic platform. When additional aliquots of the four discrepant NPW specimens were extracted and tested on both platforms, one specimen was positive on the conventional platform, and none was positive on the digital microfluidic platform. Thus, a total of three specimens were positive and the agreement between platforms for the 59 samples was 98%. The results are shown in Table 10. None of the NPWs from healthy individuals was positive on either instrument. The spiked non-patient NPWs did not inhibit the PCR; that is there was no inhibition or significant increase in the Ct of detection (data not shown). The time required to run 50 PCR cycles on the digital microfluidic platform was 60 min versus 165 min on the conventional platform.

TABLE 10
Comparison of real-time PCR results of acute patient NPWs
on conventional and microfluidic real-time PCR platforms

Conventional
realtime PCR

| | | Positive | Negative |
|---|---|---|---|
| Microfluidic real- | Positive | 2 | 0 |
| time PCR | Negative | 1 | 56 |

7.4 Field Programmable Platform for Sample-to-Sequence Identification of Pathogens

Identification of pathogens in the environment is one of the most challenging problems in diagnostics. Environmental samples are “dirty” both literally and in terms of the complexity of the analysis. It is estimated that only a fraction of organisms in an environmental sample are cultivable in media and therefore “known” to biologists. A large number of studies are currently underway to identify the complete panorama of microbes in all environmental niches. However, sequencing entire genomes of microbes in an environmental sample that is a complex mixture of microbes is a challenging undertaking both technologically and computationally.

The invention provides a digital microfluidics platform for automated sample-to-sequence identification of pathogens. Using digital microfluidics technology, the microfluidics platform of the invention provides the ability to rapidly and efficiently identify known and unknown pathogens (e.g., unidentified microbes and/or bio-engineered microbes) in a sample, such as an environmental sample. The microfluidic platform includes a multi-well droplet actuator in combination with one or more peripheral devices (e.g., sample preparation system, reagent management unit). The microfluidic platform integrates several molecular technologies on the droplet actuator (e.g., sample preparation, nucleic acid amplification, subtractive hybridization and sequencing) for automated sample-to-sequence capabilities in a small and robust device. The integrated approach use combinations of sub-pooled primers and hierarchical PCR amplifications for template preparation which are specifically designed to enrich unknown DNA sequences from unknown pathogens and categorize DNA sequences from known pathogens. Each integrated molecular technology may be represented as a different module on the droplet actuator.

Because of the software programmability of digital microfluidics, essentially all of the parameters varied between and within different modules, such as incubation times, sequences of reagent additions, washing protocols and thermal programs, may be configured on a single droplet actuator. Di vital microfluidics also provides the ability to run independent, parallel sequencing reactions so that time-to-answer may be arbitrarily scaled down. Ultra-long sequencing reads may also be performed for rapid assembly of DNA sequences and pathogen identification. Software updates may be readily implemented on the digital microfluidic platform to provide up-to-date sequence information of identification of known and unknown pathogenic organisms.

In one embodiment, subtractive hybridization may be used to deplete signatures of known pathogens from a complex sample that includes both known and unknown organisms, effectively enriching the unknown sequences.

In another embodiment, a pooling strategy may be used to minimize the number of reagent wells, whereby careful selection of sub-pools of primers in r×2N reagent wells yields an N^{r} target discrimination after r rounds of PCR amplification.

In yet another embodiment, a sequencing strategy may be used wherein the flexibility of digital microfluidics provides for ultra-long contiguous sequencing reads that are rapidly obtained on template DNA by “blind-filling” regions with nucleotide triplets. The sequencing strategy may, for example, include pyrosequencing which may be used to sequence both the conserved and variable regions of the amplified genes from the complex sample mixture.

7.4.1 Microfluidics Platform

The digital microfluidics platform of the invention may include a control module, a droplet actuator cartridge interface, and a detection module. The modules may be integrated into a single self-contained sample-to-sequence instrument unit that is suitable for on-site (e.g., in the field) use. Sample-to-sequence processes performed on a droplet actuator include sample preparation, multiple tier multiplexed real-time PCR, selection and clonal amplification of targets, and high-throughput long-read pyrosequencing.

In one example, the control module may be based on existing hardware control architecture, such as that of Advanced Liquid Logic, Inc. The architecture includes standard interfaces for communication with peripheral devices as well as a defined set of op-codes for most digital microfluidic tasks.

The cartridge interface may include thermal, magnetic, mechanical, optical, fluid and electrical interfaces to the droplet actuator cartridge. In one configuration, the cartridge is inserted into a deck in which magnets and heaters are applied from the bottom side and electrical connection and optical detection occur on the other side. The arrangement of these external components may be changed to accommodate the specific design and requirements of the cartridge.

The detection module may, for example, be a single photomultiplier tube (PMT) module that is mounted directly above the cartridge. In one example, detection of individual reactions on the droplet actuator cartridge may be by time-division multiplexing where droplets are sequenced through a single detection window. In another example, an array detector may be used to image on or more reactions on a droplet actuator cartridge and increase throughput of the analysis. In another example, a photodiode array or a charge-coupled device (CCD) imager may be used.

7.4.2 Pathogen Detection Strategy

The pathogen detection strategy of the invention combines hierarchical PCR amplification and rapid long read sequencing for detection of known and unknown pathogens. In one embodiment, the detection strategy is based on the premise that sequencing the conserved ancient elements of the replicative machinery such as a 1,500 base sequence of the 16S and 28S ribosomal DNA (rDNA) sequences may be used to identify most bacterial and fungal pathogens. Sequencing of the variable regions of the 16S and 28S rDNA is widely accepted as the most robust and versatile method for identification of microbes in a complex environment where there are mixtures of organisms^{24, 25}. The sequences that flank the variable regions of the 16S rDNA sequences in bacteria and 28S rDNA in fungi are highly conserved and may be used to amplify the variable regions of the sequences from all organisms, both known and unknown. These sequences show a great degree of homology in related organisms and may be used to make taxonomic identification. There are several projects underway to identify the uncultivable bacteria and fungi from every imaginable niche in the environment^{26, 27}. These studies have demonstrated that there are vast numbers of microbes yet to be identified and serve as a powerful tool to identity organisms that are “unknown” to the scientific community.

Because there is extensive conservation of rDNA sequences among closely related species and enough variability between genera, a two-pronged PCR amplification approach may be used to separate the sequencing of “known” versus truly “unknown” sequences. An example of an amplification approach includes the following:

1) Generic amplification of 16S and 28S rDNA using degenerate primers to amplify all genomic material of known and unknown organisms (degenerate pool);

2) Specific amplification of 16S and 28S sequences using more stringent homologous primers from known pathogens for rapid identification of known organisms (known pool); and

3) Enrichment of unknown organism sequences by subtractive hybridization of the amplified products of the degenerate pool from the known pool.

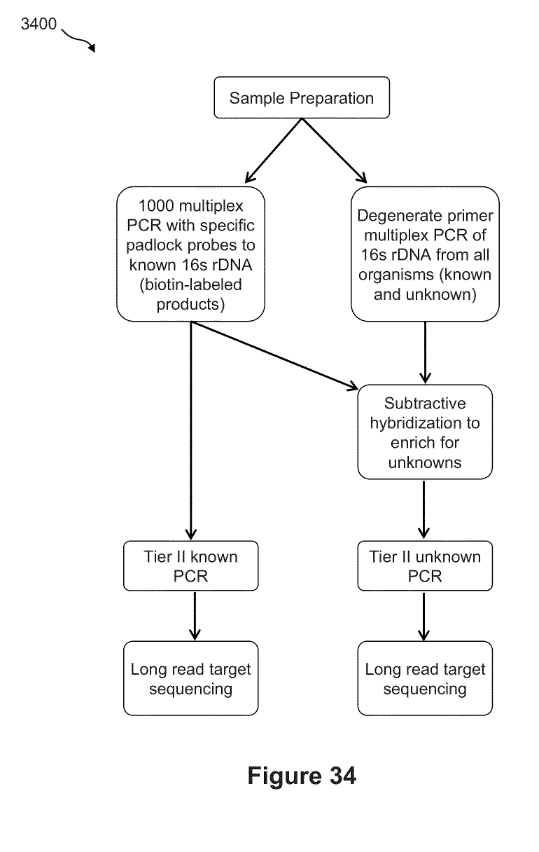

FIG. 34 illustrates a flow diagram of an example of a microfluidic protocol 3400 for detection of known and unknown pathogens on a droplet actuator. Microfluidic protocol 3400 uses different processing modules that are integrated on the droplet actuator. For example, a sample preparation module may be integrated into a droplet actuator that is configured for multiplexed PCR amplification (PCR module) of known and unknown target sequences. A subtractive hybridization module for enrichment of unknown pathogen sequences may be integrated into a droplet actuator that is configured for multiplexed PCR amplification. A sequencing module (e.g., pyrosequencing) for rapid, long read target sequencing may be integrated into a droplet actuator that is configured for multiplexed PCR amplification.

Microfluidic protocol 3400 for detection of known and unknown pathogens may include, but is not limited to, the following steps.

In one step, nucleic acids (e.g., DNA and/or RNA) in an environmental sample are isolated, purified and concentrated in a sample preparation module. In one embodiment, the sample preparation module may be integrated with the droplet actuator. In another embodiment, the sample preparation module may be integrated on the droplet actuator.

In another step, target nucleic acid sequences (e.g., 16S rDNA sequences) of all known and unknown pathogens are amplified in a PC module configured for multiplexed amplification. In this step, degenerate primers are used to amplify both known and unknown 16S rDNA sequences from all organisms in a sample (degenerate pool).

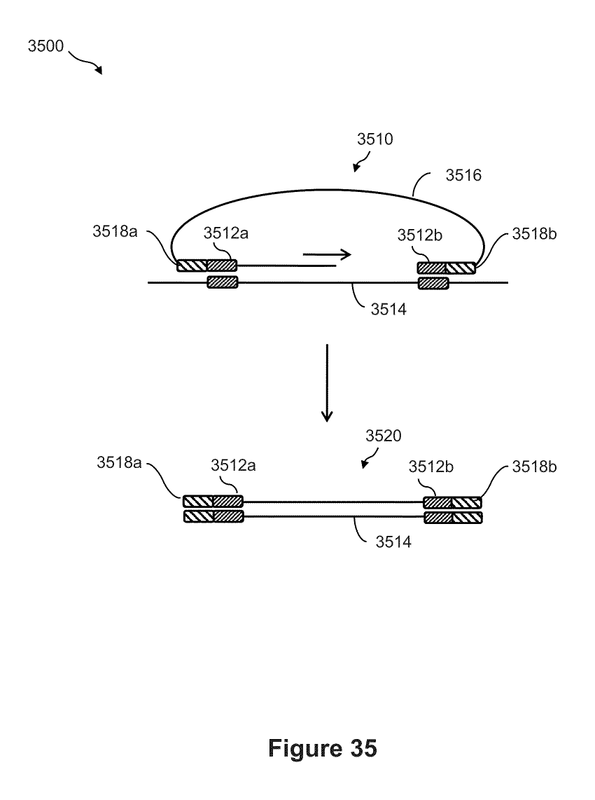

In another stop, target nucleic acid sequences (e.g., 16S rDNA sequences) of known pathogens are amplified in a PCR module configured for multiplexed amplification. In this step, biotinylated primers with a high degree of homology to known pathogens and more stringent amplification parameters are used to identify known pathogens in a sample (i.e., known pool). In one example, specific DNA probes, such as padlock probes (i.e., Spacer Multiplex Amplification (SMART) technology²⁸), may be used to amplify known sequences of 16S rDNA as described in reference to FIG. 35. The PCR products in the known pool are biotinylated to enable capture of the amplicons on magnetically responsive beads, such as streptavadin-coated magnetic beads, for subsequent manipulations on the droplet actuator.

In another step, 16S rDNA sequences amplified from unknown organisms in the degenerate pool may be enriched by subtractive hybridization using biotinylated amplicons from the known pool. In this step, sequences amplified using specific padlock probes (i.e., known pool) and immobilized on magnetically responsive beads are hybridized to sequences amplified from all organisms using degenerate primers to deplete the “unknown pool” of known sequences. Streptavadin-coated magnetically responsive beads may be efficiently mixed in an oscillating droplet on the microfluidic platform enabling rapid hybridization which will remove targets that are complementary to the known biotinylated amplicons. Subtractive hybridization may be used to efficiently and effectively enrich for low levels of unknown pathogen sequences in a complex sample.

In another step, the sub-pools of enriched unknown sequences and the known sequences are amplified in one or more additional rounds of PCR amplification to identify which pools contain amplified product. A series of hierarchical amplifications using a subdivided set of pooled probes are used to identify known microorganisms, as described with reference to FIG. 28. Hierarchical PCR may generate specific PCR products that may be taken directly to sequencing for definitive identification without the need for massive sampling of sequences for unknown sequences. The pool of enriched “unknown” sequences may be subjected to “single-molecule” amplification by diluting out the PCR products and reamplifying them in droplets (clonal amplification).

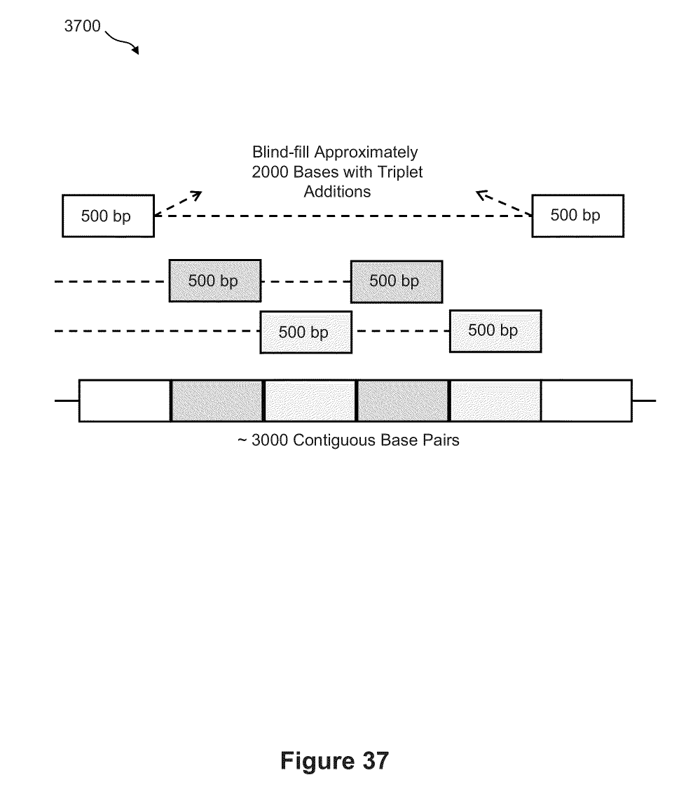

In another step, amplified known and unknown sequences are transported to a sequencing module where long read target sequencing is performed. In this step, an array of sequencing reactions and blind fill may be used to achieve, for example, >3000 bp reads, as described with reference to FIG. 37.

In another embodiment, amplification other conserved genes may be used to identify microorganisms.

In yet another embodiment, pools of probes may be designed to solely amplify all sequences encoding known toxins, virulence factors and other agents that may be engineered into otherwise benign organisms. In this example, amplification of sequences encoding known pathogenic factors eliminates the need for long read sequencing of the putatively benign organism genome.

Because the of the software programmability of the digital microfluidic platform, the balance between additional tiers of PCR amplification and sequencing may be readily adjusted and optimized. The diagnostic criteria may be expanded as additional information on threat agents becomes available.

FIG. 35 illustrates a flow diagram of an example of a process 3500 of using padlock probe technology for amplification of related nucleic acid sequences. Padlock probes (i.e., the SMART amplification protocol) are designed to amplify thousands of related, unique targets in a single reaction²⁸. In one example, SMART amplification may be used to amplify and identify one or more known pathogens in an environmental sample. SMART amplification may be readily incorporated into a pathogen detection protocol on a digital microfluidic platform.

A padlock probe 3510 includes a pair of probe sequences 3512a and 3512h that target a region of interest 3514 on DNA (e.g., genomic DNA). Probe sequences 3512a and 3512b are tethered together by a long linking molecule (padlock), such as tether 3516, which is a tether of DNA. Tether 3516 is common to all padlock probes 3510 that target various DNA sequences. Padlock probe 3510 also includes a pair of amplification primer sequences 3518a and 3518b. Amplification primer sequences 3518a and 3518b are common to all padlock probes 3510. A quantity, e.g., thousands, of padlock probes 3510 may be mixed together in a single reaction.

In solution, padlock probes 3510 hybridize to the target sequences they bracket, i.e., DNA region of interest 3514. Padlock probe 3510 is extended from one end, e.g., primer sequence 3518a (direction of arrow). When the extended sequence reaches the other end, e.g., primer sequence 3518b, the completed sequence is ligated. Single-stranded padlock probe 3510 is now a circle. All unhybridized padlock probes 3510 (i.e., linear single strands) may be removed by digestion with exonucleases. Circular padlock probe 3510 is then amplified using common primers to sequences 3518a and 3518h in a multiplex amplification to yield amplicons 3520. Common primers used for amplification may, for example, be biotinylated to yield biotinylated amplicons that may be immobilized on magnetically responsive beads.

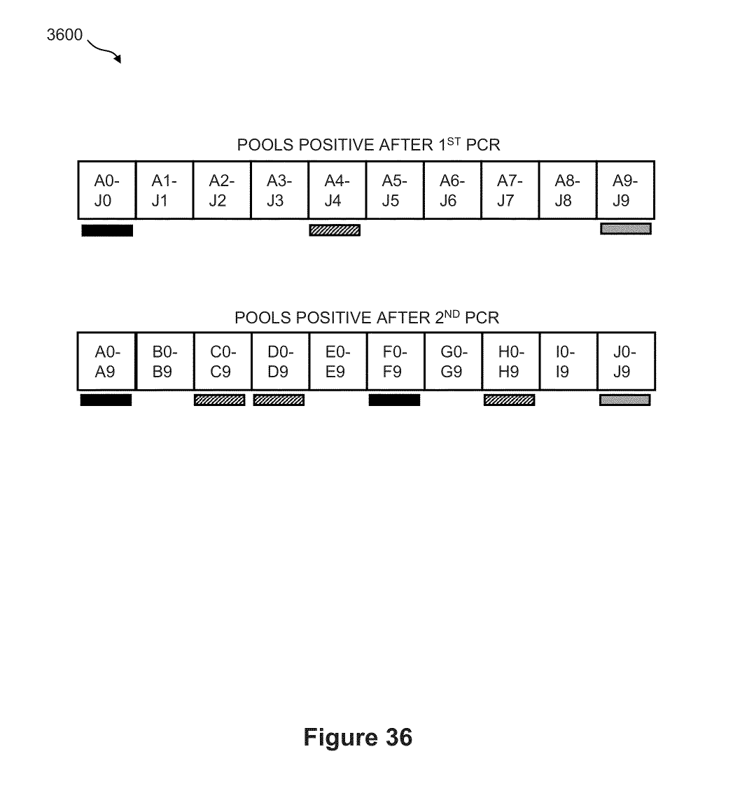

The identification of known organisms uses a series of hierarchical amplifications (SMART technology). In one example, padlock probes to about 1000 known microorganisms may be constructed and distributed into 10 carefully selected and informative pools of 100 primers for multiplex PCR amplification. The target sequences may be selected such that they amplify only closely related species. The mixtures of probes may be used to amplify the rDNA sequences from microorganisms in an environmental sample. Because the amplifications are performed with pools of probes, only a few pools may have amplification products. Amplicons from the first tier amplification may be used as template for a second tier PCR using a subdivided sot of pooled probes to narrow down the identification process. FIG. 36 illustrates a flow diagram of an example of a simplified PCR matrix description 3600 using 20 sub-pools of 100 primers for SMART amplification. Each of the 10 wells in a second tier amplification also contains 100 primers pre-selected as a 10% subset from each of the prior wells. The second tier amplification may provide a 100-fold discrimination of the identity of the organisms in the original sample. Referring to FIG. 36, individual hits (positive amplifications) on individual primers A1, F1, C4, D4, H4 and A9 were identified after two rounds of PCR amplification.

7.4.3 PCR Amplification and Sequencing Modules

The mien, die platform of the invention includes a droplet actuator that integrates several molecular technologies (i.e., nucleic acid amplification, subtractive hybridization and sequencing) for multiplexed analysis of pathogen nucleic acids on a single droplet actuator. The droplet actuator may include an arrangement of droplet operations electrodes (e.g., electrowetting electrodes) that may be scaled (i.e., adjusted in size) to accommodate the complexity of the integrated molecular technologies and multiplexed analysis. In one example, droplet operations electrodes may be 1.125 mM electrodes and progress to 50 to 250 micron electrodes.

In one embodiment, the integrated droplet actuator has an operating droplet volume of about 100 nL and may support about 10-20 simultaneous template preparation, amplification, and sequencing reactions.

In another embodiment, the integrated droplet actuator has an operating droplet volume in the picoliter range. Reduction in operating droplet volume may be achieved by using improved PCB manufacturing processes for fabrication of the bottom substrate of the droplet actuator. Because improved PCB manufacturing processes are used, finer features may be patterned on the PCB substrate and the density of sequencing reactions may be increased to about 500 to 1000 reactions. The metal line width is the primary determinant of the minimum electrode size and therefore of the minimum possible droplet size and maximum electrode arrangement density. Examples of suitable improved manufacturing processes include additive metallization, use of flexible circuit substrates and microvias formed by laser-drilling.

The size of the droplet actuator cartridge may also be increased to accommodate a larger number and scale of operations. For example, the number of droplet operations lanes, the number of PCR reaction loops and the size of the sequencing matrix may be increased. In another example, one or more different droplet actuator cartridges with different pathogen identification programs may be used in a device modified to accommodate more than one droplet actuator cartridge.

Reduction of reaction volumes may also yield other performance advantages, such as faster reaction kinetics, faster mixing and increased operating speeds. Optimization of sequencing chemistry and microfluidic sub-processes, such as bead washing protocols, may also be used to increase single read lengths, for example, to about 1000 bp within about 1 hr.

In another embodiment, long read length sequencing protocol may be extended to about 3000 bp in about 1 hr. FIG. 37 illustrates a flow diagram of an example of a protocol 3700 to increase long road sequencing to 3000 bp in about 1 hour. Protocol 3700 uses the ability of the droplet actuator to perform multiple independent reactions in parallel. Three droplets (represented by each sequence line) in independently controlled reaction lanes may be operated in parallel. By adding and washing a mix of three nucleotides at a time (e.g., the triplets ACG, ACT, CGT, GTA), arbitrarily long regions of sequence may be extended between two shorter sequencing reads (indicated by dashed line). The extended sequence is approximate, but statistically may be predicted with sufficient accuracy. A contiguous 3000-bp sequence read may then be reconstructed.

7.4.4 Integration of Sample Preparation and Analysis

Preparation of nucleic acids (e.g., DNA and/or RNA) from an environmental sample may be performed on-bench or directly on a droplet actuator. In one embodiment, a commercially available on-bench sample preparation system (i.e., nucleic acid extraction system) may be integrated with a droplet actuator configured for DNA amplification and sequencing to provide sample-to-sequence capability. The sample preparation system may be selected based on the composition of the samples to be evaluated and the interfacing requirements of the droplet actuator. A preferred format for a sample preparation system uses magnetically responsive beads for capturing nucleic acids in a sample. Suitable examples of sample preparation systems include NUCLISENS® MINIMAG® (available from bioMérieux, Inc. Durham, N.C.), Qiagen BIOROBOT® EZ1 Workstation (available from QIAGEN Inc., Valencia, Calif.), and Arcxis Biotechnologies XISYL™ (available from Arcxis Biotechnologies, Pleasanton, Calif.).

Magnetically responsive beads for capturing nucleic acids in a sample are the preferred format because they are directly compatible with digital microfluidic protocols. Both silica-coated and charge-switch magnetic beads may be used for nucleic acid extractions on a droplet actuator. Concentration of magnetic beads may be performed directly in cartridge wells followed by capture of the beads within a single dispensed droplet and washing of the beads to purify and elute DNA on the droplet actuator. The sample preparation system may be used for cell lysis, removal of particulates, and pre-concentration to reduce the sample volume from milliliters to 100 μL without loss of sensitivity. Because the droplet actuator may accept samples deposited directly in open wells, the fluidic interface between an on-bench sample preparation system and the droplet actuator may be readily achieved. In one example, an input sample volume of about 0.1 mL to about 1.0 mL may be loaded onto the droplet actuator. Additional concentration and extraction may be performed on the droplet actuator.

The digital microfluidic platform also includes a high-performance microprocessor and built-in capabilities for communicating with and controlling multiple peripheral devices to provide the required operational integration between systems. In addition to a sample preparation system, other peripheral devices such as a reagent management unit may be integrated with the droplet actuator. Typically, reagents may be stored in wells formed within the top substrate of a droplet actuator cartridge. Off-cartridge reagent storage, such as in a reagent management unit, may be used to accommodate an increased reagent load and run-time.

In another embodiment, a sample preparation module may be integrated on a droplet actuator. In this example, liquid handling for sample preparation may be performed using digital microfluidics to enable substrate-level integration (e.g., bottom substrate of a droplet actuator) of all processes. Although the sample preparation and analysis modules (e.g., amplification, subtractive hybridization and sequencing models) are integrated on the same substrate (e.g., bottom substrate) the scale of each module may be substantially different. For example, the sample preparation module may be designed to manipulate relatively large volumes of fluid (e.g. 10's μL) while the analysis modules may operate on smaller droplets (e.g., 100 nL or less). Because the droplet actuator (i.e., electrowetting system) is unpressurized, interfacing the sample preparation module and analysis modules is relatively straightforward. Macro-volume samples may be delivered to intermediate processing wells where they are discretized into numerous discrete droplets for downstream processing, (e.g., analysis). Numerous discrete droplets may also be collected into an intermediate processing, well to form a larger volume for sample preparation or analysis.

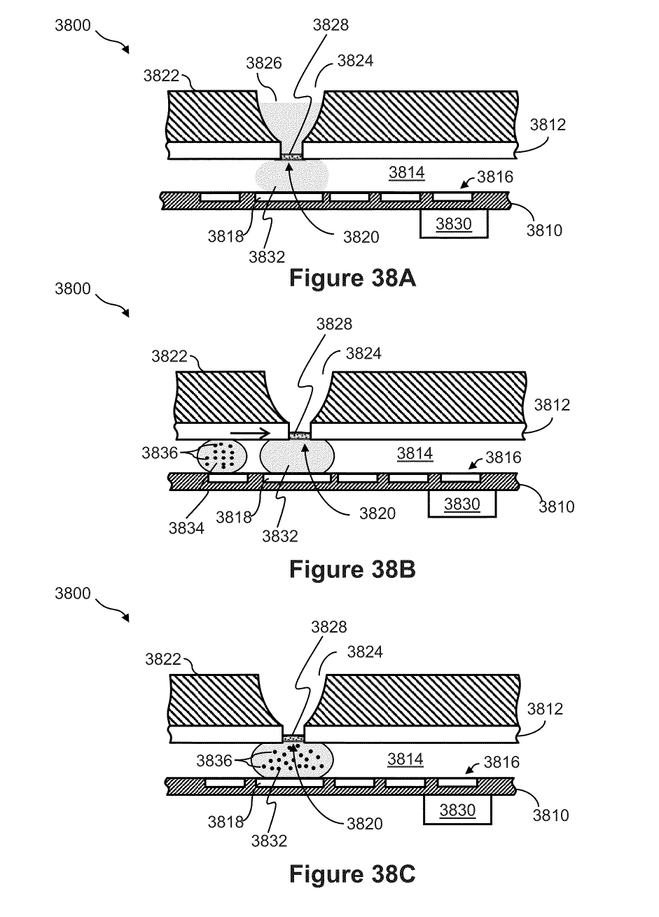

FIGS. 38A, 38B and 38C illustrate side views of an example of a portion of a droplet actuator 3800 and show a process of integrating sample preparation on a droplet actuator. Droplet actuator 3800 may include a bottom substrate 3810 that is separated from a top substrate 3812 by a gap 3814. An arrangement of droplet operations electrodes 3816 (e.g., electrowetting electrodes) and a dispensing electrode 3818 may be disposed on bottom substrate 3810. Droplet operations are conducted atop droplet operations electrodes 3816 on a droplet operations surface. An opening 3820 may be provided within top substrate 3812. Opening 3820 is substantially aligned with dispensing electrode 3818. Dispensing electrode 3818 may be aligned with an internal processing well (not shown). A substrate 3822 may be disposed atop top substrate 3812. Substrate 3822 may include a well 3824, which is suitable for delivering a large volume of liquid through opening 3820 and into gap 3814. Well 3824 contains a quantity of fluid 3826. Fluid 3826 may, for example, be a sample fluid combined with a lysis buffer. A filter 3828 is positioned between well 3824 and the internal processing well aligned with dispensing electrode 3818. Filter 3828 my, for example, be a lateral or vertical flow filter. A magnet 3830 is arranged in close proximity to droplet operations electrodes 3816. In particular, magnet 3830 is arranged such that a certain droplet operations electrodes 3816 (e.g., droplet operations electrode 3816M) is within the magnetic field thereof. Magnet 3830 may, for example, be a permanent magnet or an electromagnet.

An example of a process of preparing a DNA sample from an environmental sample may include, but is not limited to, the following steps.

In one step, FIG. 38A shows a sample lysis protocol in which an environmental sample is combined with a volume of lysis buffer in well 3824. The combined sample and lysis buffer solution is incubated for a period of time sufficient to yield a lysed cell solution (lysate) 3832 that contains released nucleic acids (e.g., DNA). Lysate 3832 flows through filter 3828 and into the internal well aligned with dispensing electrode 3818 by means of capillary action. As lysate 3832 flows through filter 3828, cellular debris and particulates are removed.

In another step, FIGS. 38B and 38C show a DNA recovery process in which a reagent droplet 3834 that includes a quantity of magnetically responsive beads 3836 is combined using droplet operations with lysate 3832. Magnetically responsive beads 3836 may, for example, be magnetic charge-switch DNA purification beads. Lysate 3832 with magnetically responsive beads 3836 therein is incubated for a sufficient period of time for released DNA to bind magnetically responsive beads 3836. Magnetically responsive beads 3836 are then concentrated into one or more individual droplets (not shown) for subsequent processing. One or more DNA capture droplets (not shown) may be transported using droplet operations into the magnetic field of magnet 3830 and washed using a merge-and-split wash protocol to remove unbound material, yielding a washed bead-containing droplet substantially lacking in unbound material (not shown). The purified DNA is then eluted from magnetically responsive beads 3836 with elution buffer. The eluted DNA contained in the droplet surrounding the magnetically responsive beads 3836 may then be transported away from the beads for further processing on the droplet actuator, e.g., for execution of a droplet based PCR amplification protocol and pyrosequencing.

7.4.5 Sample Analysis

The genomic complexity of a sample to be analyzed may dictate the selection of an analysis program. For example, for samples with low genomic complexity, an analysis program that routes PCR products directly to the sequencing matrix may be used. For samples with high genomic complexity, selection of individual amplicons (clonal amplification) from may be required prior to sequencing. In this example, the analysis program may include a droplet dilution protocol to first obtain a very dilute sample. The diluted sample may be routed through several cycles of real-time PCR to determine at which dilution an amplification signal is detected. Once no signal is detected for the most dilute sample, successively more concentrated samples may be routed through the PCR module until a signal is detected, at which point the PCR cycles may continue until saturation is observed. Once saturation is observed, the PCR mixture may be routed to the sequencing matrix. The real-time PCR amplification signal may be used by a programmed algorithm to select the optimum routing strategy.

7.4.6 Integrated On-Chip Pyrosequencing

On-bench protocols for template preparation and pyrosequencing may be described and implemented on a droplet actuator as discrete step-by-step droplet-based protocols. Some modifications to existing assay protocols facilitate translation of the bench-based protocols into droplet-based protocols.

The droplet actuator may be designed to fit onto an instrument deck that houses extra-droplet actuator features such as one or more magnets for immobilization of magnetically responsive beads and one or more heater assemblies for controlling the temperature within certain reaction and/or washing zones.

7.4.7 Translation of an On-Bench Template Preparation Protocol

Template preparation protocols may, for example, include PCR amplification of target DNA, binding of amplified double stranded DNA to magnetically responsive beads, denaturation of immobilized double stranded DNA, and annealing of single stranded target DNA to pyrosequencing printers.

Candida albicans (C. albicans) genomic DNA (ATCC #10231-D) was used as the template for PCR amplification. The primer pair used to amplify a 211-bp fragment of C. albicans DNA was: forward primer 5′ GAA ACG ACG CTC AAA CAG (fluorescent label) 3′ and reverse primer 5′ (biotin label) ATG CTT AAG TTC AGC GGG TA 3′. The pyrosequencing primer was FAM pyo #5 5′ TGC TTG AAA GAC GGT ACT GG 3′).

The on-bench protocol was as follows. PCR was performed in a 100 μL reaction volume that included 1× Platinum Taq PCR buffer, 0.2 mM dNTPs, 3 mM MgCl₂, 10 pg/μL C. albicans genomic DNA, 0.05 U of Platinum Taq DNA polymerase (Invitrogen), and 0.2 μM of each primer. Activation of Taq polymerase was initiated by a 5 minute hot start at 75° C. followed by 40 cycles of amplification using the BIO-RAD IQ5 instrument. Each cycle comprised a denaturation step at 95° C. for 30 seconds, a primer annealing step at 55° C. for 30 seconds, and a chain-elongation step at 72° C. for 30 seconds. A final elongation step at 72° C. was performed to ensure complete extension of the amplified DNA. The PCR product was purified using a Qiagen PCR purification kit. The initial volume of the 100 μL PCR product was eluted into 50 μL of binding buffer (10 mM Tris-HCL pH 7.6, 2 M NaCl, 1 mM EDTA, 0.1% TWEEN® 20). The reaction was performed in duplicate. One of the duplicates was used to verify amplification of the target DNA and estimate the concentration (about 52 ng/μL) of PCR product by gel electrophoresis. The other purified DNA sample was used in subsequent template processing steps for pyrosequencing. In these steps, the purified DNA was immobilized on 50 μL of Streptavidin M280 DYNABEADS® beads (10 μg/μL). The beads were first washed three times in binding buffer and resuspended into 50 μL final volume of binding buffer. The optimal binding capacity for 50 μg of beads is 1.7 μg of PCR product, therefore 33 μL of PCR product at about 52 ng/μL was added to the beads. DNA and beads were incubated at 65° C. for 15 minutes with periodic mixing to resuspend the beads. Following binding of the DNA to the beads, the DNA was denatured using 0.5 M NaOH. For this step, beads were incubated in 100 μL 0.5 M NaOH for 1 minute and then washed one time in 0.5 M NaOH. Beads with denatured DNA (single stranded DNA; ssDNA) thereon were washed three times in Mag-annealing buffer (20 mM Tris-Acetate pH 7.6, 5 mM Mg-Acetate) and resuspended in a final volume of 50 μL. Mag-annealing buffer. Supernatant from 10 μL of the washed beads was removed and 80 μL of Mag-annealing buffer was added to resuspend the processed beads. FAM Pyro #5 primer (3.8 μL of 10 μM FAM Pyro #5 primer) was then added to the bead suspension and beads and primer were incubated for 2 minutes at 80° C. and subsequently allowed to cool to room temperature. The beads with template/primer hybrids bound thereon were washed 3 times with pyre wash buffer (50 mM Tris Acetate pH 7.6, 0.5 mM EDTA, 5 mM Mg Acetate, 100 mM NaCl, 1 mM DTT, 0.01% TWEEN® 20) and then resuspended in a final volume of 10 μL. Single stranded binding protein was added to the washed bead suspension at a concentration of 5 μg for 1 μg DNA. Fluorescence detection was used to determine the concentration of bound DNA at 38.95 pmols of DNA/1 μL of beads.

Fluorescent measurements were taken throughout the on-bench template preparation protocol. A standard curve was made using the FAM pyre #5 primers mixed with beads. Measurements were done on a 96 well plate using the plate reader parameters of a gain #75, 485/20 and 528/20. The results are shown in Table 11. The data indicate that the DNA was present on the beads, then denatured, and then FAM primer was bound. The concentration of the final sample was approximately 39 pmol/μL.

TABLE 11
Fluorescent reads throughout on-bench template preparation
ssDNA with FAM Sequencing Primer

| | Concentration | | |
|---|---|---|---|
| Fluorescence | (pmol/2 ul) | Fluorescence w/o zero | |
| 117 | 0 | 0 | |
| 134 | 0.1 | 17 | |
| 225 | 1 | 108 | |
| 494 | 10 | 377 | |
| 1681 | 100 | 1564 | |
| 7281 | 1000 | 7164 | |
| 1091 | 55.26499356 | 974 | FAM ds DNA |
| 172 | −14.41428463 | 55 | ds DNA no FAM |
| 160 | −15.32413375 | 43 | ssDNA |
| 138 | −16.99219046 | 21 | ssDNA |
| 1390 | 77.93540071 | 1273 | ss DNA w/FAM primer |
| 138 | −16.99219046 | 21 | 3rd wash |

Pyrosequencing template preparation on-chip (PCR-C01 cartridge) was performed using a protocol that paralleled the on-bench protocol. Modifications to the protocol included the following: A serial dilution bead washing protocol was used instead of the buffer exchange wash procedures used in the on-bench protocol. A wash step using binding buffer was added to the protocol between the DNA denaturization and primer annealing steps to remove excess NaOH prior to the buffer exchange into Mag-annealing buffer.

FIG. 39 illustrates a top view of an example of a droplet actuator 3900 (PCR-C01) that is suitable for use in conducting a pyrosequencing template preparation protocol. In this example, the top substrate of droplet actuator 3900 may be a glass plate. The filler fluid may be either 0.01% Triton X 2 cst silicone oil or 0.005% SPAN® 85 2 cst silicone oil (available from Sigma-Aldrich Co., St. Louis, Mo.).

Droplet actuator 3900 includes multiple fluid reservoirs 3910 (e.g., 8 fluid reservoirs 3910a through 3910h), which my, for example, be allocated as waste fluid collecting reservoirs or fluid dispensing reservoirs. The eight fluid reservoirs 3910 may be arranged in order from 3910a through 3910h. In this example, a first fluid reservoir 3910a was used as a waste fluid collecting reservoir for receiving spent reaction droplets; fluid reservoirs 3910b through 3910g were used as reagent dispensing reservoirs dispensing pyro wash buffer, FAM pyro #5 primer, Mag-annealing buffer, NaOH, binding buffer, and beads (5 μg/μL), respectively; fluid reservoir 3910h was used to dispense purified DNA. Fluid reservoirs 3910 are interconnected through an arrangement, such as a path or array, of droplet operations electrodes 3912 (e.g., electrowetting electrodes).

Droplet operations are conducted atop droplet operations electrodes 3912 on a droplet operations surface. Droplet actuator 3900 may include a temperature change zone 3914 and a washing zone 3916. A magnet 3918 (e.g., a permanent magnet or electromagnet) may be located in proximity to (e.g., underneath) washing zone 3916. Magnet 3918 may be embedded within the deck that holds droplet actuator 3900 when it is mounted on the instrument (not shown). Magnet 3918 is positioned in a manner which ensures spatial immobilization of magnetically responsive beads during bead washing protocols. Droplet actuator 3900 may further include a detection spot 3920.

Droplet actuator 3900 is designed to fit onto an instrument deck that houses extra-droplet actuator features such as magnet 3918 for immobilization of magnetically responsive beads, one or more heater assemblies (e.g., two heater bars) for controlling the temperature within certain processing zones, and a 4× fluorimeter.

The template preparation protocol performed on droplet actuator 3900 (PCR-C01) included the following steps: Purified dsDNA prepared on-bench using the PCR protocol described above was loaded onto the on-chip sample dispensing reservoir. Control droplets (2× droplets) of dsDNA, binding buffer, and pyro wash buffer were dispensed and transported to the detection spot for determination of background fluorescence. A 1× bead droplet (5 μg/μL Streptavidin M280 Dynabeads) was dispensed and combined with a 1× binding buffer droplet to yield a 2× bead droplet that was used for determination of background bead fluorescence. After fluorescence determination, the 2× bead droplet was washed using a merge and split bead washing protocol. The 2× bead droplet was combined using droplet operations with a 2× binding buffer wash droplet to yield a 4× bead/wash droplet. The 4× bead/wash droplet was split using droplet operations to yield a 2× washed bead droplet and a 2× supernatant droplet. The 2× supernatant droplet was transported to the detection spot and fluorescence determined. The merge and split wash protocol was repeated once. The 2× washed bead droplet was split using droplet operations to yield a 1× bead droplet and a 1× supernatant droplet A 1× dsDNA sample droplet was dispensed and combined using droplet operations with the 1× bead droplet to yield a 2× dsDNA/bead droplet. Following an incubation period (e.g., 15 minutes) sufficient for binding of DNA to the beads, the DNA was denatured using a NaOH wash protocol. Voltage and frequency settings were adjusted to facilitate dispensing and manipulation of 0.5 M NaOH droplets. A 2× NaOH droplet (0.5 M NaOH) was dispensed and transported to the detection spot for determination of background fluorescence. A second 2× NaOH droplet was dispensed and combined using droplet operations with the 2× dsDNA head droplet to yield a 4× denaturation droplet. After a period of time sufficient (e.g., about 30 seconds) for denaturation of the dsDNA, the 4× denaturation droplet was split using droplet operations to yield a 2× ssDNA/bead droplet and a 2× supernatant droplet. The 2× supernatant droplet was transported to the detection spot and fluorescence determined. The NaOH wash was repeated twice. The 2× ssDNA/bead droplet was washed twice with binding buffer using the bead washing protocol to remove excess NaOH. The 2× ssDNA/bead droplet was washed 3 times with Mag-annealing buffer using the bead washing protocol to exchange the droplet buffer to Mag-annealing buffer. A 2×FAM primer droplet (FAM pyro #5 primer) was dispensed and combined using droplet operations with the 2× ssDNA/bead droplet to yield a 4× ssDNA/bead/primer droplet. The 4× ssDNA/bead/primer droplet was incubated for 2 minutes at 80° C. for primer annealing and subsequently allowed to cool to room temperature. The 4× ssDNA/bead/primer droplet was split using droplet operations to yield a 2× annealed DNA/bead droplet and a 2× supernatant droplet. The 2× annealed DNA/bead droplet was washed 3 times using the bead washing protocol with pyro wash buffer to remove excess unbound primers.

Eight separate reactions were performed using the pyrosequencing template preparation protocol. Seven reactions were performed on droplet actuators using 0.01% Triton X 2 cst silicone oil as the filler fluid. One reaction was performed on a droplet actuator using 0.005% SPAN® 85 2 cst silicone oil as the filler fluid. NaOH dispensing and transport may be more efficient in 0.005% SPAN® 85 2 cst silicone oil. Fluorescent measurements (twelve separate measurements) were taken throughout the protocol to monitor the FAM labeled DNA and primers. In order to prevent bead loss during droplet transport, wash (supernatant) droplets were read for fluorescence instead to the bead-containing sample droplet. The sequence of fluorescent measurements is shown in Table 12. Fluorescence data is shown in Table 13.

TABLE 12
On-chip fluorescent reads in order of occurrence

| Read Number | Sample |
|---|---|
| 1* | DNA |
| 2* | Binding Buffer |
| 3* | Pyro Wash |
| 4* | Beads |
| 5 | Binding Buffer wash 1 |
| 6 | Binding Buffer wash 2 |
| 7* | NaOH |
| 8 | NaOH 1 |
| 9 | NaOH 2 |
| 10 | NaOH 3 |
| 11 | Pyro Wash |
| 12 | Final Sample |

*Measurements are control reads. Except for the final sample (number 12), the other non control reads are of supernatant droplets split from the beads. All fluorescent measurements were performed using 2X droplets.

TABLE 13
Fluorescence data from PCR-C01 template preparation

| | 22-Jul | 27-Jul | 27-Jul | 28-Jul | 28-Jul | 29-Jul | 30-Jul | 30-Jul | 31-Jul |
|---|---|---|---|---|---|---|---|---|---|
| Chip # | 4366 | 4363 | 4363 | 4360 | 4360 | 4490 | 4489 | 4489 | 4488 |
| DNA* | 265 | 516 | 518 | 289 | 297 | 303 | 702 | 332 | 294 |
| BB* | 192 | 412 | 429 | 201 | 244 | 218 | 603 | 233 | 212 |
| PW* | 188 | 418 | 414 | 199 | 198 | 217 | 598 | 238 | 211 |
| Beads* | 155 | 262 | 273 | 171 | 191 | 156 | 113 | 169 | 164 |
| NaOH* | 193 | 355 | 365 | 199 | 211 | 221 | 628 | 213 | 208 |
| BBW1 | 193 | 343 | 368 | 207 | 213 | 215 | 651 | 231 | 215 |
| BBW2 | 193 | 351 | 375 | 208 | 220 | 219 | 635 | 231 | 213 |
| NaOH 1 | | 404 | 413 | 241 | 240 | 221 | 629 | 216 | 215 |
| NaOH 2 | 224 | 386 | 424 | 225 | 225 | 222 | 651 | 343 | 288 |
| NaOH 3 | 209 | 382 | 402 | 209 | 218 | 227 | 619 | 331 | 244 |
| PW | 212 | 447 | 428 | 212 | 215 | 242 | 595 | 228 | 234 |
| Sample | 237 | 361 | 454 | 227 | 219 | 241 | 499 | 244 | 233 |
| | | | | | | | everything | | |
| | | | | | | | minus | | |
| | | | | | | | 1000 | | |

*Measurements are control reads.

FIG. 40 shows an example of a plot 4000 of the fluorescence data of the PCR-C01 samples of Table 13 that were collected from the droplet actuator and pooled together for pyrosequencing. The data show reactions for 22-Jul and 28-Jul have a distinct fluorescence pattern that was repeated across all three reads. The same pattern at a higher fluorescence is also seen in the 27-Jul samples. The pattern of the 30-Jul samples do not conform to the exact same pattern (i.e., the pattern is elevated) due to misalignment of the droplet actuator on the instrument deck. Sample 29-Jul (not shown) had a flattened pattern and therefore was not collected for pyrosequencing. The data also indicate the denaturation of the dsDNA occurred during the first wash with NaOH, which was about 30 seconds.

The eight samples (8 droplets of beads at about 5 μg/μL) prepared using the template preparation protocol described in reference to FIGS. 39 and 40 were collected, pooled together and processed on-bench. The pooled sample was washed with pyro wash buffer and suspended in 2 μL of buffer for pyrosequencing on a second droplet actuator (PCR-D01; not shown). SSB was added to the sample on-bench. The on-chip pyrosequencing protocol was performed as described with reference to FIGS. 13A-13D and 14A-14C.

FIG. 41 shows an example of a histogram 4100 of on-chip pyrosequencing results of 13-bp sequenced on a 211-bp long C. albicans DNA template. Circled dNTPs are background measurements. Assuming the bead concentration was 7 μg/μL the amount of DNA loaded onto the beads can be calculated using the fluorescent reads from pyrosequencing. These results were calculated to be approximately 50 fmol/μg DNA on beads. The initial concentration of DNA was 730 fmol/μL; therefore, about ⅓ of the DNA bound to the beads during template preparation on-chip. This result is consistent with the results seen on the bench.

7.4.8 Integrated PCR, Template Preparation and Pyrosequencing On-Chip

The template preparation protocol described in reference to droplet actuator 3900 (PCR-E01) of FIG. 39 was further adapted into a program for integrating template preparation and pyrosequencing on a single droplet actuator.

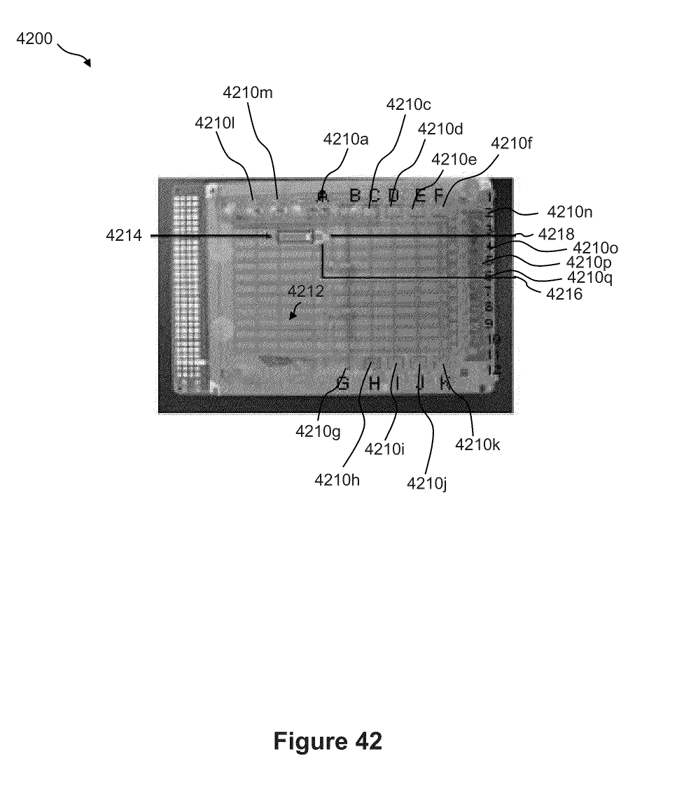

FIG. 42 illustrates a top view of an example of a droplet actuator 4200 (PCR-E01) and shows an example layout of fluid reservoirs for collecting and dispensing fluids for integrated template preparation and pyrosequencing reactions. In this example, the top substrate of droplet actuator 4200 is a glass plate. A reservoir plate is positioned atop the top substrate. The filler fluid is 0.005% SPAN® 85 2 cst silicone oil.

Droplet actuator 4200 includes multiple fluid reservoirs 4210 (e.g., 17 fluid reservoirs 4210a through 4210q), which may, for example, be allocated as waste fluid collecting reservoirs or fluid dispensing reservoirs. In this example, fluid reservoir 4210g was used as a waste fluid collecting reservoir for receiving spent reaction droplets; fluid reservoirs 4210a, 4210k and 4210m were used as reagent dispensing reservoirs for dispensing pyro wash buffer; fluid reservoirs 4210c through 4210f were used as reagent dispensing reservoirs for dispensing dATP, dCTP, dGTP, and dTTP, respectively, for pyrosequencing reactions; fluid reservoirs 4210n through 4210q were used as reagent dispensing reservoirs for dispensing beads, NaOH, FAM pyro #5 primer, SSB, respectively, for template preparation reactions; fluid reservoirs 4210h and 4210i were used as reagent dispensing reservoirs for dispensing Mag-annealing buffer and binding buffer, respectively; fluid reservoir 4210j was used as a reagent dispensing reservoir for dispensing Mellow enzyme mix for pyrosequencing reactions; and fluid reservoir 4210l was used as a reagent dispensing reservoir for dispensing PPi detection mix for pyrosequencing. Fluid reservoirs 4210 are interconnected through an arrangement, such as a path or array, of droplet operations electrodes 4212 (e.g., electrowetting electrodes). Droplet operations are conducted atop droplet operations electrodes 4212 on a droplet operations surface. Droplet actuator 4200 may include a temperature change zone 4214 and a washing zone 4216. A magnet 4218 (e.g., a permanent magnet or electromagnet) may be located in proximity to (e.g., underneath) washing zone 4216. Magnet 4218 may be embedded within the deck that holds droplet actuator 4200 when it is mounted on the instrument (not shown). Magnet 4218 is positioned in a manner which ensures spatial immobilization of magnetically responsive beads during bead washing protocols.

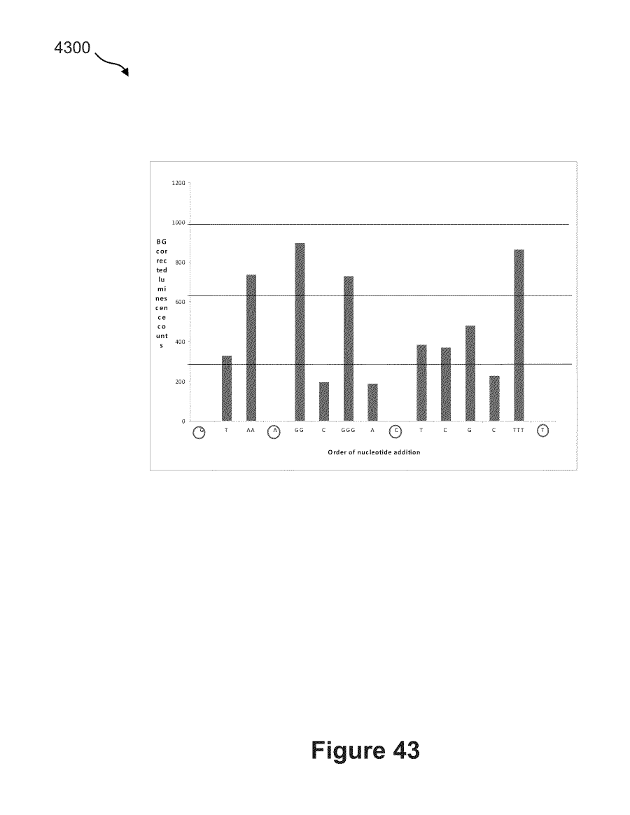

FIG. 43 shows an example of a histogram 4300 of pyrosequencing results of 17-bp sequenced on a 211-bp long C. albicans DNA using a protocol that integrates template preparation and pyrosequencing on the same droplet actuator. Circled dNTPs are background measurements. The template preparation protocol used was substantially the same as the template preparation protocol described in reference to FIG. 39 except for a few modifications. All fluorescent measurements performed during the template preparation protocol were eliminated. Two 1× bead droplets (10 μg/μL) were combined and incubated in tandem with two 1× droplets of DNA (2× dsDNA/bead droplets). The incubation period for dsDNA binding to beads was decreased from 15 minutes to 5 minutes. The 2× dsDNA/bead droplets were merged using droplet operations into a 4× dsDNA/bead droplet. The 4× dsDNA/bead droplet was split using droplet operations into a 2× dsDNA/bead droplet and a 2× supernatant droplet. At the end of the template preparation protocol, the 2× annealed DNA/bead droplet was combined on-chip with a 2× droplet that contains SSB protein. The 2× annealed DNA/bead droplet is ready for pyrosequencing. A pyro wash protocol was run to wash the chip prior to pyrosequencing. During the pyro wash protocol, the 2× annealed DNA/bead droplet was positioned at a specific droplet operations electrode outside the wash area. The pyrosequencing protocol as described with reference to FIGS. 13A-13D and 14A-14C was performed without any further changes.

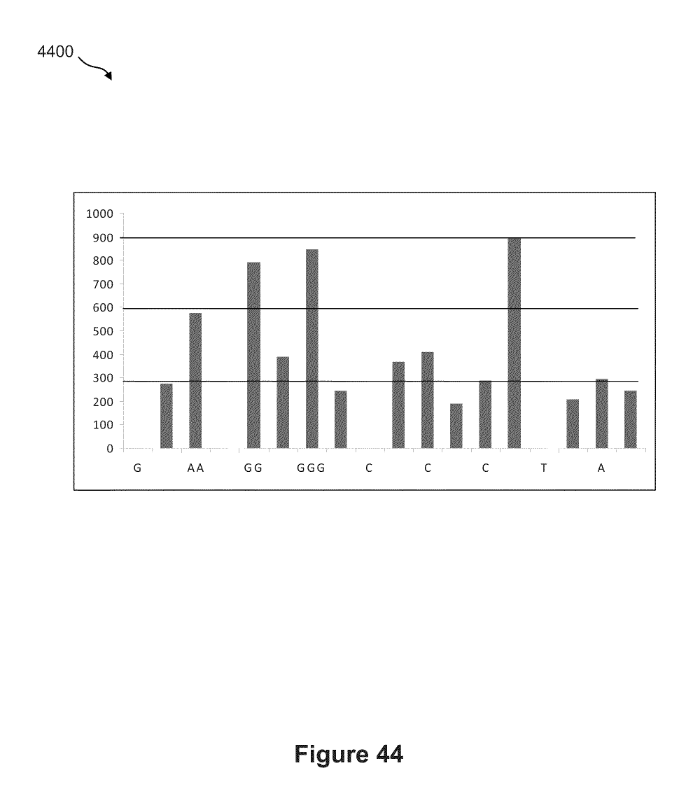

A two temperature PCR protocol was adapted to the template preparation and pyrosequencing program described in reference to FIGS. 42 and 43. FIG. 44 shows an example of a histogram 4400 of pyrosequencing results of 20-bp sequenced on a 211-bp long C. albicans DNA using a protocol that integrates PCR, template preparation, and pyrosequencing on the same droplet actuator. The PCR protocol uses a PCR master mix that includes a hot-start Taq DNA polymerase. The composition of the PCR master mix is shown in Table 14.

TABLE 14
PCR master mix for PCR on-chip
Amount of Mix 30 ul

| | Initial | | Final | | Amount |
|---|---|---|---|---|---|
| Reagent | Concentration | | Concentration | | in μL |
| PCR buffer | 10 x | | 1 x | | 3 |
| dNTPs | 10 | mM | 0.2 | mM | 0.6 |
| MgCl2 | 50 | mM | 3 | mM | 1.8 |
| Fwd FAM | 10 | μM | 1 | μM | 3 |
| primer | | | | | |
| Rv Biotin primer | 10 | μM | 1 | μM | 3 |
| Platinum Taq | 5 | μ/μL | 0.5 | μ/μL | 3 |
| Genomic candida DNA | 250 | pg/μL | 10 | pg/μL | 1.2 |
| Water | | | | | 14.4 |

The PCR protocol included the following steps: The components of the PCR master mix were combined on-bench and loaded onto a dispensing reservoir of a droplet actuator. Two 2× droplets of PCR mix were dispensed in tandem onto two separate reaction lanes of the droplet actuator. The 2×PCR droplets were transported using droplet operations to certain droplet operations electrodes within a temperature control zone at 96° C. After a 2 minute incubation at 96° C. (hot start), the droplets were cycled between two temperature control zones (96° C. and 57° C.) for 40 cycles of denaturation at 96° C. for 10 seconds and a 30 second annealing step at 57° C. A final annealing step was performed for 1 minute at 57° C. At the completion of the PCR protocol, one 2×PCR droplet was removed from the droplet actuator for verification of DNA amplification by gel electrophoresis not shown). The second 2×PCR droplet was split using droplet operations into two 1× droplets and combined with two separate 1× bead droplets for initiation of the template preparation protocol and subsequent pyrosequencing.

Purification of the PCR product prior to template preparation was not required because competition for streptavidin binding sites was expected to be minimal. According to bead product specifications, 650-900 pmol/mg free biotin can bind to beads. In this PCR protocol, beads were used at a concentration of 10 μg/μL. Therefore, there are 6.5 pmol of binding sites per μL beads. The concentration of primer was 1 pmol/μL primer, which results in a ratio of 1 primer to 6.5 binding sites. There are 730 fmol dsDNA 211 bp in 100 ng/μL. If the initial concentration of primers was 1000 fmol/μL, then the final concentration of primers after PCR is 270 fmol/μL. During bead binding, the DNA to primer ratio is about 3:1 allowing for minimal competition for streptavidin binding sites.

7.5 Bead Immobilization

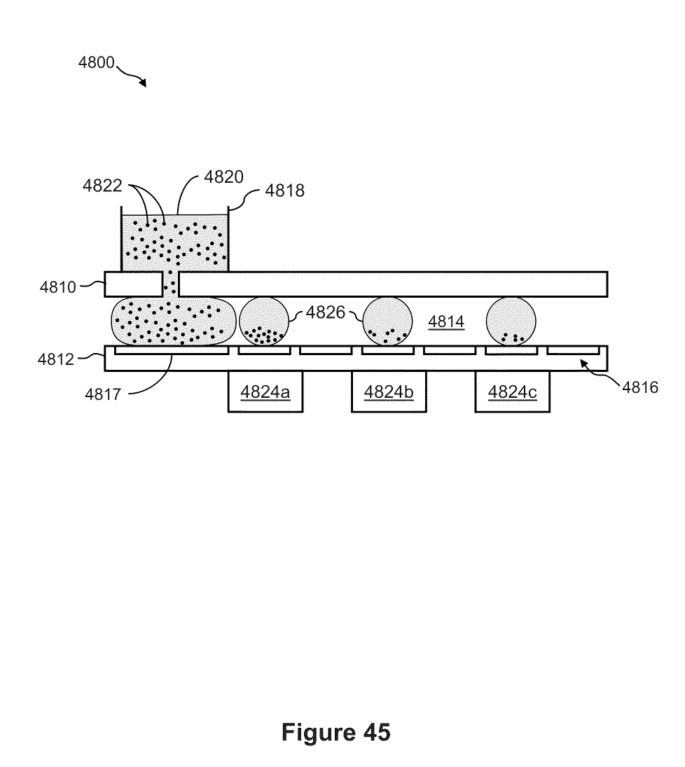

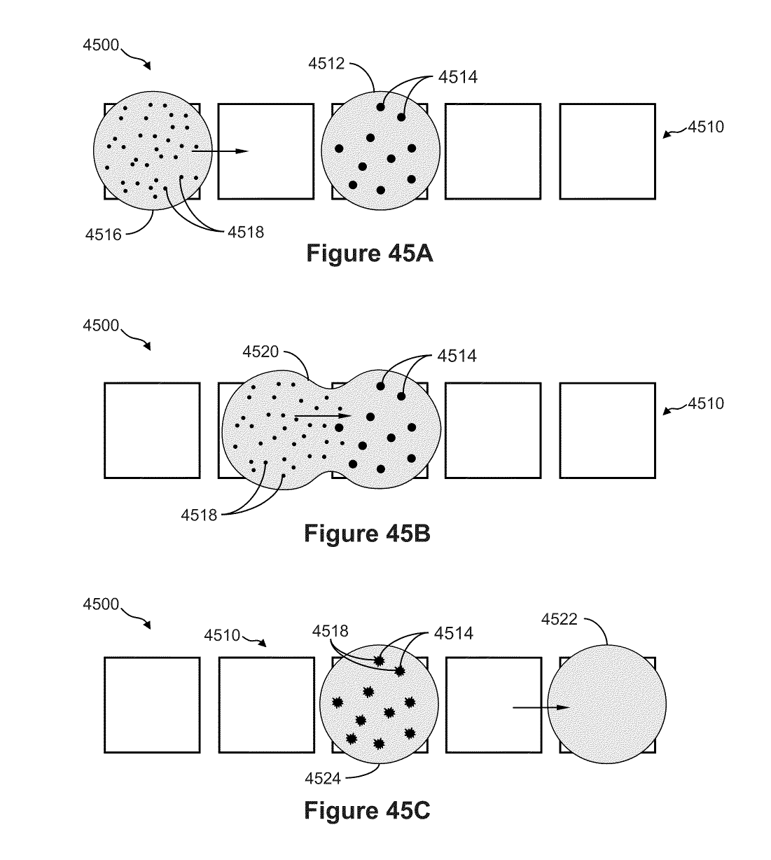

FIGS. 45A through 45C illustrate top views of an example of a portion of an electrode arrangement 4500 of a droplet actuator (not shown) and show a process of bead immobilization using magnetic forces that are provided directly inside a droplet. Electrode arrangement 4500 may include an arrangement of droplet operations electrodes 4510 (e.g., electrowetting electrodes). Droplet operations are conducted atop droplet operations electrodes 4510 on a droplet operations surface. The process of immobilizing paramagnetic beads may be useful, for example, when irreversible aggregation is acceptable or required, such as in a separation process in Which the supernatant is of interest while the solid phase is not of interest.

Referring to FIG. 45A, a bead collecting droplet 4512 is provided at a certain droplet operations electrode 4510. Bead collecting droplet 4512 is a fluid droplet that has magnetic beads 4514 dispersed therein. Magnetic beads 4514 are not to be confused with magnetically responsive beads. That is, magnetic beads 4514 are formed fully or in part of magnetic material for providing a magnetic force. In one example, magnetic beads 4514 are beads that have micro-magnets incorporated therein for providing the magnetic force. An example of micro-magnets that are suitable for Conning magnetic beads 4514 is the Micro-Magnet™ product (available from BJA Magnetics, Leominster, Mass.; www.bobjohnsonassociates.com/html/micro-magnet.html). In this example, the Micro-Magnet™ product is formed of Neodymium Iron Boron (NdFeB) with energy products up to 52 Megagauss Oersted (MGOe). The Micro-Magnet™ product is coated with an inert biocompatible protective coating.

In another example, magnetic beads 4514 are beads that have ferromagnetic materials incorporated therein for providing the magnetic force. An example of a ferromagnetic material that is suitable for forming magnetic beads 4514 is SPHERO™ ferromagnetic particles (available from Spherotech, Inc., Lake Forest, Ill.; www.spherotech.com). In this example, the SPHERO™ ferromagnetic particles are prepared using chromium dioxide coated onto uniform polystyrene particles. These particles retain magnetism once exposed to a magnetic field. The particles can be demagnetized and re-magnetized repeatedly and reproducibly. Therefore, in this example, the fields may be reversed to demagnetize them for reversible attraction.

Referring again to FIG. 45A, a sample droplet 4516 is provided at a certain droplet operations electrode 4510. Sample droplet 4516 is a fluid droplet that has paramagnetic beads 4518 dispersed therein. Paramagnetic beads 4518 are beads that have paramagnetic material incorporated therein for providing a magnetic force only when in the presence of an externally applied magnetic field. More specifically, paramagnetism is a form of magnetism that occurs only in the presence of an externally applied magnetic field. Unlike ferromagnets, paramagnets do not retain any magnetization in the absence of the externally applied magnetic field. An example of a method of bead immobilization and, in particular, a process of immobilizing paramagnetic beads on a droplet actuator may include, but is not limited to the following steps.

In one step, FIG. 45A shows the paramagnetic bead-containing sample droplet 4516 being transported via droplet operations along droplet operations electrodes 4510 and toward the magnetic head-containing bead collecting droplet 4512. In this step, the paramagnetic beads 4518 of sample droplet 4516 are not in the presence of any magnetic field and, thus, are not magnetized.

In another step, FIG. 45B shows the paramagnetic bead-containing sample droplet 4516 being merged with the magnetic bead-containing bead collecting droplet 4512 using droplet operations. Once the droplet merging process begins, the paramagnetic beads 4518 of sample droplet 4516 move into the magnetic fields of magnetic beads 4514 of bead collecting droplet 4512 and become magnetized. The merging of sample droplet 4516 and bead collecting droplet 4512 form a merged droplet 4520 in which the paramagnetic beads 4518 are attracted to the magnetic beads 4514. That is, paramagnetic beads 4518 become magnetized and retain their magnetism because they are in the presence of the magnetic fields of magnetic beads 4514. In this way, the paramagnetic beads 4518 are immobilized at the surfaces of magnetic beads 4514 of the now merged droplet 4520.

In yet another step, FIG. 45C shows that a substantially bead free droplet 4524 is split away from merged droplet 4520 of FIG. 45B using droplet operations. This is because substantially all paramagnetic beads 4518 are immobilized at magnetic beads 4514 and, thus, are left behind in a droplet 4524 that contains substantially all of the paramagnetic beads 4518 and magnetic beads 4514. In this way, beads, such as paramagnetic beads 4518, may be separated from the original sample droplet 4516. Because the invention provides a magnet (e.g., magnetic beads 4514) that is very close to the target beads (e.g., paramagnetic beads 4518) the attraction and separation process is achieved with exceptionally good results. Essentially, the invention provides a magnet inside a droplet in a droplet actuator.

7.6 Bead Washing by Filtration

Current washing techniques rely mostly on the use of magnetically responsive beads. However, in certain applications non-magnetically responsive beads may be present. For example, an assay may have already been prepared using non-magnetically responsive beads and it may be inconvenient to switch the type of beads in the assay. The use of confinement structures to wash “large” non-magnetically responsive beads has been implemented for washing beads that are comparable in size to the droplet actuator gap height. However, this process is not suitable for washing “small” beads, which may be any beads that are a certain amount smaller than the gap height. The invention provides filters in a droplet actuator for performing a bead washing process that is suitable for use with substantially any sized beads.

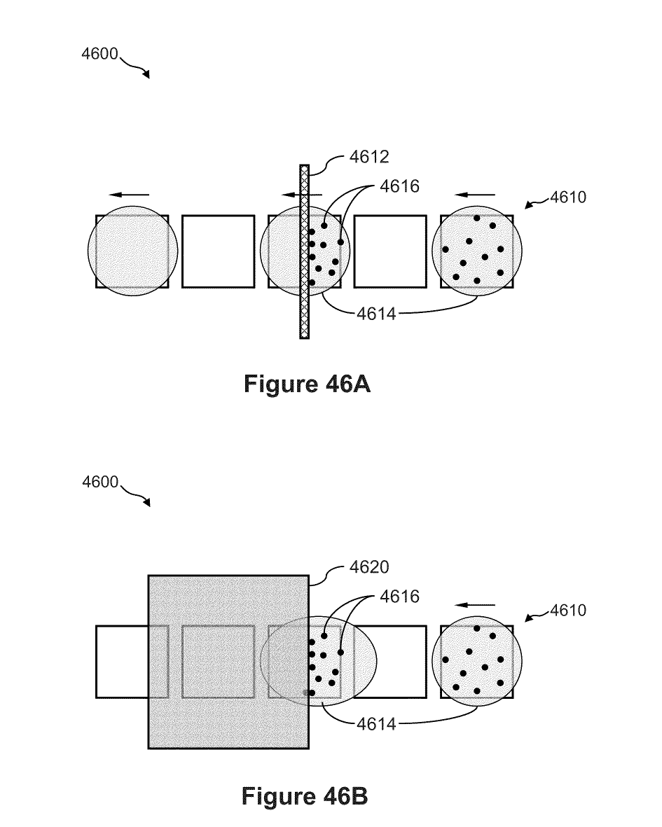

FIGS. 46A and 46B illustrate top views of an example of a portion of an electrode arrangement 4600 of a droplet actuator (not shown) and show examples of bead washing by filtration. Electrode arrangement 4600 may include an arrangement of droplet operations electrodes 4610 (e.g., electrowetting electrodes). Droplet operations are conducted atop droplet operations electrodes 4610 on a droplet operations surface.

In one embodiment and referring to FIG. 46A, a filter strip 4612 is provided at a certain droplet operations electrode 4610. In one example, the mesh size of filter strip 4612 is suitable for retaining small beads (e.g., 3-6 micron beads). In this example, filter strip 4612 may be placed between the two substrates (not shown) of the droplet actuator to draw off liquid while at the same time retaining the beads at a certain droplet operations electrode 4610. In one embodiment, filter strip 4612 may be a strip whose width is about equal to or less than the width of droplet operations electrodes 4610. Liquid may be transported into contact with filter strip 4612 and then removed on the other side of filter strip 4612 to wash the beads. For example FIG. 46A shows droplets 4614 that have beads 4616 dispersed therein. As droplets 4614 are being transported along droplet operations electrodes 4610 and come into contact with filter strip 4612, beads 4616 are retained against filter strip 4612. At the same time, liquid passes through filter strip 4612.

Filter strip 4612 may be formed, for example, of a “string” of filter material that is slightly larger than the gap height of the droplet actuator. The string of filter material is compressed between the two substrates. In this case, the width of the filter is preferably slightly larger than the gap height, which enables easy transport of the liquid fully across the filter. This implementation is thirty straightforward to manufacture as the string of filter material is simply pulled tight, aligned with the droplet actuator, and then compressed or glued into place. Clogging of the filter may be avoided because it only needs to exclude the micron sized beads.

A filter, such as filter strip 4612, may also be incorporated into the droplet actuator for routine filtering of sample and reagent materials. That is, all droplets may be forced through a filter before entering a certain area of the droplet actuator to mitigate against particulate contamination. Further, a droplet actuator may include stages of two or more filters that exclude different sized particles.

In another embodiment and referring to FIG. 46B, filter strip 4612 of FIG. 46A is replaced with a filter area 4620 covering one or more droplet operations electrodes 4610. That is, filter area 4620 may be substantially larger than a typical droplet operations electrode 4610 in order to draw off essentially all the liquid surrounding the beads. The filter area 4620 may be repeated as many times as needed (i.e. adding fresh liquid each time) until finally the beads are removed into a fresh droplet of liquid and transported away.

7.7 Improve Assay Throughputs Using Phase-Change Filler Fluids and Droplet Immobilization

Currently, the instrumentation of microfluidic systems is fully occupied throughout every single assay. Droplet operations mediated by activated electrodes may be used to retain reaction droplets in place for incubation, which is not very efficient for assays with a long incubation step. For example, a newborn screening assay requires an 8-hour incubation step. In this example, one instrument is able to run only one assay per day. The invention provides a method of increasing throughput by executing the incubation step off-instrument with reaction droplets immobilized in the removable droplet actuator by phase-change filler fluid. An example of a method of increasing throughput by executing the incubation step off-instrument with reaction droplets immobilized by phase-change filler fluid may include, but is not limited to, the following steps.

In one step, the droplet actuator is mounted in the instrument and droplet operations are performed to, for example, dispense and mix samples/reagents. During these droplet operations the droplet actuator is heated to about 30˜40° C. and the oil (e.g., filler fluid) is kept in liquid phase.

In another step, after all the reaction droplets are made and located, the droplet actuator is cooled to room temperature. As a result of cooling, the oil solidifies and holds all the droplets in place in the droplet actuator.

In yet another step, the droplet actuator is removed from the instrument and set aside for the incubation period, in the example of the newborn screening assay, the droplet actuator is set aside outside of the instrument for the 8-hour incubation step. This frees up the instrument and allows the instrument to be used for the next droplet actuator/assay. Therefore, each droplet actuator/assay only needs a short time on the instrument and can be batch incubated off-instrument.

In yet another step, after the long incubation period is completed, the droplet actuator is remounted into the instrument for final signal reading. This process may be performed for any number of droplet actuators/assays in a given day.

Another advantage of the method of the Invention is that the surfacing fouling during incubation may be reduced because the oil film between the droplet and Cytop coating is solidified and non-breakable.

7.8 Double Magnet Washing Configuration

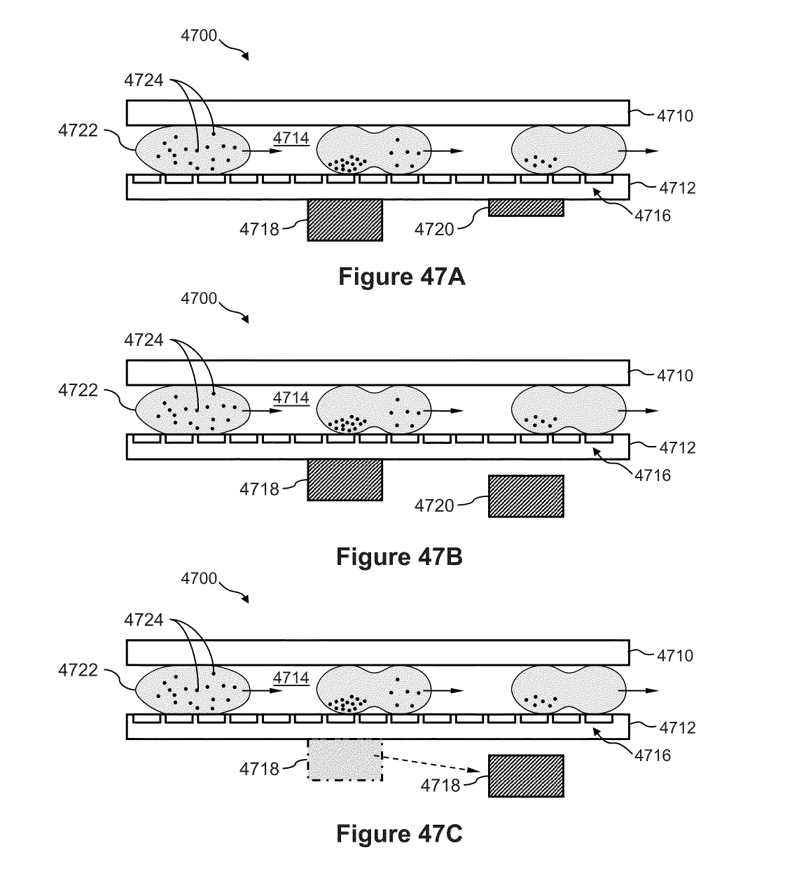

FIGS. 47A through 47C illustrate side views of an example of a portion of a droplet actuator 4700 and show examples of applying two different magnetic field strengths for bead washing, Droplet actuator 4700 may include a bottom substrate 4710 and a top substrate 4712 that are separated by a gap 4714. Bottom substrate 4710 may include a path or array of droplet operations electrodes 4716 (e.g., electrowetting electrodes). Droplet operations are conducted atop droplet operations electrodes 4716 on a droplet operations surface.

FIG. 47A shows a first magnet 4718 that is positioned in close proximity to a certain droplet operations electrode 4716 of droplet actuator 4700. Additionally, a second magnet 4720 is positioned in close proximity to another droplet operations electrode 4716 of droplet actuator 4700. First magnet 4718 and second magnet 4720 may be permanent magnets or electromagnets. In one example, first magnet 4718 has a greater magnetic field strength than second magnet 4720.

First magnet 4718, which has a greater magnetic field strength than second magnet 4720, may be used to remove hulk supernatant by snapping off a droplet including a first portion of the beads as a droplet is transported away from first magnet 4718. For example, FIG. 47A shows a droplet 4722 that is transported using droplet operations along droplet operations electrodes 4716. Droplet 4722 includes a certain concentration of magnetically responsive beads 4724. At first magnet 4718, a portion of the droplet 4722 may snap off leaving a certain amount of magnetically responsive beads 4724 behind at the droplet operations electrode 4716 that is neat first magnet 4718. Very high dilution factors per wash can be achieved using this method. Therefore, a second stage washing with the weaker second magnet 4720 may be used where beads can be dispersed better to wash the interstices.

In another embodiment and referring to FIG. 47B, first magnet 4718 and second magnet 4720 may have substantially the same magnetic field strengths. However, first magnet 4718 is positioned closer to droplet operations electrodes 4716 than second magnet 4720. In this way, two different magnetic field strengths may be present at droplet operations electrodes 4716. That is, the closer the magnet the stronger its effect on the beads.

In yet another embodiment and referring to FIG. 47C, a single moveable magnet (e.g., moveable first magnet 4718) may be used to supply the two different magnetic field strengths depending on the location of the single magnet. In this example, the magnet is moveable laterally along droplet operations electrodes 4716 as well as vertically to achieve different spacing. Further, the movement of the single magnet is coordinated with the movement of the droplet via droplet operations along droplet operations electrodes 4716.

Referring again to FIG. 47A, 47B, or 47C, a process of bead washing by applying two different magnetic field strengths may be as follows. In a first step, a droplet splitting operation is performed in the presence of the magnetic field of first magnet 4718. In doing so, a portion of droplet 4722 that includes a portion of the magnetically responsive beads 4724 is retained at first magnet 4718 and the other portion of the droplet 4722 is split off that also includes a portion of the magnetically responsive beads 4724. This field strength at first magnet 4718 is sufficiently high to prevent all of the beads from being transported away. The second step invokes performing a droplet splitting operation in the presence of the magnetic field of second magnet 4720 to yield a droplet substantially lacking in beads and a droplet including substantially all of the remaining beads that were not split off in the first step. Note that in the droplet splitting operations, because the splitting is mediated by electrodes, at least three droplet operations electrodes 4716 may be activated to spread the droplet across the three electrodes, then an intermediated electrode is deactivated causing the droplet to split. Additionally, after the droplet splitting operations, the resulting two or more bead-containing droplets may be recombined to yield a droplet comprising substantially all of the beads that were present in the original droplet. A main aspect of this process is that it can lead to overall retention of at least about 99% or about 99.9% or about 99.99% or about 99.999% or about 99.9999% of the beads.

7.9 Multiple Magnet Setup for Bead Concentration

Current, a certain amount of bead loss may occur during bead concentration for sample preparation. For example, current sample preparation protocols may involve a large volume (on the order of 10-250 μL) sample input. All beads from this sample are accumulated in a single droplet above a magnet. In the beginning, a single droplet is placed on an electrode above the magnet and then multiple droplets are dispensed from the sample pool by continually merging and splitting with the droplet above the magnet, leaving the beads behind (above the magnet). However, at high switching frequencies an increasing amount of beads get split into the droplet that is going into waste, which eventual causes a significant loss of beads. The invention uses multiple magnets to concentrate the beads. As a result, droplet operations may be performed at high switching frequencies without bead loss.

FIG. 48 illustrates a side view of an example of a portion of a droplet actuator 4800 and shows an example of using multiple magnets for bead concentration. Droplet actuator 4800 may include a bottom substrate 4810 and a top substrate 4812 that are separated by a gap 4814. Bottom substrate 4810 may include a path or array of droplet operations electrodes 4816 (e.g., electrowetting electrodes). Droplet operations are conducted atop droplet operations electrodes 4816 on a droplet operations surface. Additionally, a reservoir electrode 4817 is arranged along the path or array of droplet operations electrodes 4816. A fluid well 4818 is provided at top substrate 4812 and substantially aligned with reservoir electrode 4817. Fluid well 4818 may be a large volume fluid well, such as in the order of 10-250 μL. An opening in top substrate 4812 allows sample fluid 4820 to flow from fluid well 4818 into gap 4814 and atop reservoir electrode 4817. Sample fluid 4820 also includes a certain quantity of magnetically responsive beads 4822.

Multiple magnets 4824 are arranged along droplet operations electrodes 4816 such that the electrodes are within the magnet fields of the magnets 4824. In one example, magnets 4824a, 4824b, and 4824c are arranged along droplet operations electrodes 4816. In this example, magnet 4824a is arranged closest to reservoir electrode 4817, while magnet 4824c is arranged farthest from reservoir electrode 4817. Magnets 4824 may be permanent magnets or electromagnets.

During the sample preparation protocol, droplets 4826 are dispensed from sample fluid 4820 at reservoir electrode 4817 and transported along droplet operations electrodes 4816 via droplet operations. First, a single droplet 4826 is dispensed onto the droplet operations electrode 4816 at magnet 4824a. Then multiple droplets 4826 are further dispensed from sample fluid 4820 continually merging and splitting with the droplet 4826 above magnet 4824a leaving a certain amount of magnetically responsive beads 4822 behind at magnet 4824a. However, as the multiple droplets 4826 are dispensed from sample fluid 4820 and move toward magnet 4824h, any magnetically responsive beads 4822 that are not immobilized at magnet 4824a may be immobilized at magnet 4824b. Likewise, as the multiple droplets 4826 are dispensed from sample fluid 4820 and move yet further toward magnet 4824c, any magnetically responsive beads 4822 that are not immobilized at magnets 4824a and 4824b may be immobilized at magnet 4824c.

In summary, when, for example, a second or third bead concentration sequence is employed by having more than one magnet, waste droplets can be subjected to further bead concentration steps to minimize bead loss. At the end of the process, all beads from the multiple concentrated bead droplets can be collected into a single bead droplet with near negligible bead loss.

7.10 Improved Wash Buffer Composition

The invention is the use of thickeners, such as, but not limited to, glycerol, polyethyleneoxide (PEO), and polyethyleneglycol (PEG), to modify the viscosity of the wash buffer in droplet actuator applications. The addition of the thickeners to the wash buffer serves to better remove contaminants from the droplet operations surface, especially when contamination is due to signal generating beads that might be lost due to sedimentation. Therefore, the invention improves wash efficiency by decreasing the number of wash cycles needed to retrieve signal baseline. Because of the improved wash efficiency (i.e., fewer washes), faster assays and/or higher throughput may be achieved.

7.11 Systems

Referring to FIGS. 1 through 48, the invention may be embodied as a method, system, or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include some or all of the following: an electrical connection haying one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the invention may be written in an object oriented programming language such as Java, Smalltalk, C++ or the like. However, the computer program code for carrying out operations of the invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Certain aspects of invention are described with reference to various methods and method steps. It will be understood that each method step can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the methods.

The computer program instructions may also be stored in a computer-readable memory that can direct a computer or other programmable data processing apparatus to function in a particular manner, such that the instructions stored in the computer-readable memory produce an article of manufacture including instruction means which implement various aspects of the method steps.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing various functions/acts specified in the methods of the invention.

### 8 REFERENCES

1 B. S. Pace, “Renaissance of Sickle Cell Disease Research in the Genome Era”, Imperial College Press, 2007.
2 R. Frikke-Schmidt, C. F. Sing, B. U. Nordestgaard, and A. Tybjaerg-Hansen. Gender- and age-specific contributions of additional DNA sequence variation in the 5′ regulatory region of the APOE gene to prediction of measures of lipid metabolism, Hum. Genet. 115: 331-345, 2004.
3 H. H Wittrup, A. Tybjaerg-Hansen, R. Steffensen, S. S. Deeb, J. D. Brunzell, G. Jensen, and B. G. Nordestgaard. Mutations in the lipoprotein lipase gene associated with ischemic heart disease in men: The Copenhagen City heart study. Arterioscle. Thromb. Vase. Biol. 19: 1535-1540, 1999.
4 B. G. Nordestgaard, S. Abildgaard, H. H. Wittrup, R. Steffensen, G. Jensen, A. Tybjaerg-Hansen, Heterozygous lipoprotein lipase deficiency; Frequency in the general population, effect on plasma lipid levels, and risk of ischemic heart disease. Circulation 96: 1737-1744, 1997.
5 R. Frikke-Schmid C. F. Sing, B. G., Nordestgaard, R. Steffensen, and A. Tybjaerg-Hansen. Subsets of SNPs define rare genotype classes that predict ischemic heart disease. Hum. Genet. 120(6): 865-877, 2007.
6 J. Bentzen, P. Poulsen, A. Baag, and M. Fenger. Further studies of the influence of apolipoprotein B alleles on glucose and lipid metabolism. Human Biology, October 2003.
7 J. Chowdhury, G. V. Kagiala, S. Pushpakon, J, Lauzon, A. Makin, A. Atrazhev, A, Sticke, W. G. Newman, C. J. Backhouse, and L. M. Pilarski. Microfluidic platform for single nucleotide polymorphism genotyping of the thiopurine s-methyltransferase gene to evaluate risk for adverse drub events. J. Molec. Diagn. 9:521-529, 2007.
8 R. Zhang, A. Abu, H. Zhu, T. Nguyen, F. Yao, K. Xia, D. Liang, and C. Liu. SNP Cutter, a comprehensive tool for SNP PCR-RFLP assay design. Nuc. Acids Res. 33:W489-W492, 2005.
9 H. de Lencastre, D. Oliveira, and A. Tomasz. Antibiotic resistant Staphylococcus aureus: a paradigm of adaptive power. Curr. Opin. Microbiol. 10(5): 428-435, 2007.
10 J. A. Yang, D. W. Park, J. W. Sohn, and M. J. Kim. Novel PCR-restriction fragment length polymorphism analysis for rapid typing of Staphylococcal cassette chromosome mec elements. J. Clinical Microbiol. 44: 236-238, 2006.
11 C. Milheirico, D. D. Oliveira, and H. de Lencastre. Multiplex PCR strategy for subtyping the staphylococcal cassette chromosome mec type IV in methicillin-resistant Staphylococcus aureus: “SCCmec IV multiplex”. J. Antimicrobial Chemotherapy 60: 42-48, 2007.
12 C. Milheirico, D. C. Oliveira, and H. de Lencastre. Update to the multiplex PCR strategy for assignment of rues element types in Staphylococcus aureus. Antimicrobial Agents Chemotherapy 51: 3374-3377, 2007.
13 R. Pal, M. Yang, R. Lin, B. N. Johnson, et. al. An integrated microfluidic device for influenza and other genetic analyses. Lab Chip 5: 1024-1032, 2005,
14 L. A. Cooper and K. Subbarao. A simple restriction fragment length polymorphism-based strategy that can distinguish the internal genes of human H1N1, H3N2, and H5N1 influenza A viruses. J. Clinical Microbiol. 38: 2579-2583, 2000.
15 B. S. Pace, “Renaissance of Sickle Cell Disease Research in the Genome Era”, Imperial College Press, 2007.
16 R. Frikke-Schmidt, C. F. Sing, B. G. Nordestgaard, and A. Tybjaerg-Hansen. Gender- and age-specific contributions of additional DNA sequence variation in the 5′ regulatory region of the APOE gene to prediction of measures of lipid metabolism. Hum. Genet. 115: 331-345, 2004.
17 H. H Wittrup, A. Tybjaerg-Hansen, R. Steffensen, S. S. Deeb, J. D. Brunzell, G. Jensen, and B. G. Nordestgaard. Mutations in the lipoprotein lipase gene associated with ischemic heart disease in men: The Copenhagen City heart study. Arterioscle. Thromb. Vase. Biol. 19: 1535-1540, 1999.
18 B. G. Nordestgaard, S. Abildgaard, H. H. Wittrup, R. Steffensen, G. Jensen, A. Tybjaerg-Hansen, Heterozygous lipoprotein lipase deficiency; Frequency in the general population, effect on plasma lipid levels, and risk of ischemic heart disease. Circulation 96: 1737-1744, 1997.
19 R. Frikke-Schmidt, C. F. Sing, B. G., Nordestgaard, R. Steffensen, and A, Tybjaerg-Hansen. Subsets of SNPs define rare genotype classes that predict ischemic heart disease, Hum. Genet. 120(6): 865-877, 2007.
20 J. Bentzen, P. Poulsen, A. Baag, and M. Fenger. Further studies of the influence of apolipoprotein B alleles on glucose and lipid metabolism. Human Biology, October 2003.
21 J. Chowdhury, G. V. Kagiala, S. Pushpakon, J. Lauzon, A. Makin, A. Atrazhev, A. Sticke, W. G. Newman, C. J. Backhouse, and L. M. Pilarski. Microfluidic platform for single nucleotide polymorphism genotyping of the thiopurine s-methyltransferase gene to evaluate risk for adverse drub events. J. Molec. Diagn. 9:521-529, 2007.
22 R. Zhang, A. Abu, H. Zhu, T. Nguyen, F. Yao, K. Xia, D. Jiang, and C. Liu. SNP Cutter: a comprehensive tool for SNP PCR-RFLP assay design. Nuc. Acids Res. 33:W489-W492, 2005.
23 H. de Lencastre, Oliveira, and A. Tomasz. Antibiotic resistant Staphylococcus aureus: a paradigm of adaptive power. Curr. Opin. Microbiol. 10(5): 428-435, 2007.
24 J. A. Yang, D. W. Park, r. Sohn, and M. J. Kim. Novel PCR-restriction fragment length polymorphism analysis for rapid typing of Staphylococcal cassette chromosome mec elements. J. Clinical Microbiol. 44: 236-238, 2006.
25 C. Milheirico, D. D. Oliveira, and H. de Lencastre. Multiplex PCR strategy for subtyping the staphylococcal cassette chromosome mec type IV in methicillin-resistant Staphylococcus aureus: “SCCmec IV multiplex”. J. Antimicrobial Chemotherapy 60: 42-48, 2007.
26 C. Milheirico, D. C. Oliveira, and H. de Lencastre. Update to the multiplex PCR strategy for assignment of mec element types in Staphylococcus aureus. Antimicrobial Agents Chemotherapy 51: 3374-3377, 2007.
27 R. Pal, M. Yang, R. Lin, B. N. Johnson, et. al. An integrated microfluidic device for influenza and other genetic analyses. Lab Chip 5: 1024-1032, 2005.
28 L. A. Cooper and K. Subbarao. A simple restriction fragment length polymorphism-based strategy that can distinguish the internal genes of human H1N1, H3N2, and H5N1 influenza A viruses. J. Clinical Microbiol. 38: 2579-2583, 2000.
29 Chen, L.; West, J.; Auroux, Manz, A.; Day, P. J. R. Anal. Chem. 2007, 79, 9185-9190.
30 Neuzil, P.; Zhang, C.; Pipper, J.; Oh, S.; Zhuo, L. Nucleic Acids Res. 2006, 34, e77.
31 Daxboeck F, Krause R, Wenisch C (2003) Laboratory diagnosis of Mycoplasma pneumoniae infection. Clin Microbiol Infect 9:263-273.
32 Loens K, Ursi D, Goossens H, Ieven M (2003) Molecular diagnosis of Mycoplasma pneumoniae respiratory tract infections. J Clin Microbiol 41:4915-4923.
33 Kong F, Gordon S. Gilbert G L (2000) Rapid-cycle PCR for detection and typing of Mycoplasma pneumoniae in clinical specimens. J Clin Microbiol 38:4256-4259.
34 Bernet C, Garret M, de Barbeyrac B, Bébéar CM, Bonnet J (1989) Detection of Mycoplasma pneumoniae by using the polymerase chain reaction. J Clin Microbiol 27:2492-2496.
35 Dumke R, Schurwanz N, Lenz M, Schuppler M, Luck C, Jacobs E (2007) Sensitive detection of Mycoplasma pneumoniae in human respiratory tract samples by optimized real-time PCR approach. J Clin Microbiol 45:2726-2730.
36 Winchell J M, Thurman K A, Mitchell S L, Thacker W L, Fields B S (2008) Evaluation of three real-time PCR assays for detection of Mycoplasma pneumoniae in an outbreak investigation. J Clin Microbiol 46:3116-3118.
37 Cler L, Bu D, Lewis C, Euhus D (2006) A comparison of five methods for extracting DNA from paucicellular clinical samples. Mol Cell Probes 20:191-196.

### 9 CONCLUDING REMARKS

The foregoing detailed description of embodiments refers to the accompanying drawings, which illustrate specific embodiments of the invention. Other embodiments having different structures and operations do not depart from the scope of the invention. The term “the invention” or the like is used with reference to certain specific examples of the many alternative aspects or embodiments of the applicants invention set forth in this specification, and neither its use nor its absence is intended to limit the scope of the applicants invention or the scope of the claims. This specification is divided into sections for the convenience of the reader only. Headings should not be construed as limiting of the scope of the invention. The definitions are intended as a part of the description of the invention. It will be understood that various details of the invention may be changed without departing from the scope of the invention. Furthermore, the foregoing description is for the purpose of illustration only, and not for the purpose of limitation.

1-137. (canceled)

138. A method of optimizing real-time PCR, the method comprising:
(a) providing a digital microfluidics system comprising at least one signal monitor;
(b) providing a sample droplet comprising a detectable signal component;
(c) conducting a PCR reaction;
(d) monitoring intensity of the detectable signal component during thermal cycles; and
(e) initiating a next thermal cycle when the signaling component of a sample reaches a plateau.

139. The method of claim 138, wherein initiating a next thermal cycle is automated such that when the signaling component of a sample reaches a plateau the next thermal cycle is automatically initiated.

140. The method of claim 138, wherein the thermal cycle comprises annealing.

141. The method of claim 138, wherein the thermal cycle comprises extension.

142. The method of claim 138, wherein the detectable signal comprises fluorescence.

143. The method of claim 142, wherein the fluorescence comprises EVA GREEN dye.

144. The method of claim 138, wherein the digital microfluidics system comprises:
(a) a droplet actuator comprising:
(i) one or more substrates arranged to form a substantially enclosed droplet operations gap;
(ii) electrodes configured for conducting droplet operations in the droplet operations gap; and
(iii) one or more filler fluids substantially filling the droplet operations gap.

145. The method of claim 138, wherein the sample droplet further comprises:
(a) a nucleic acid template; and
(b) reagents for amplifying the nucleic acid template.

146. The method of claim 144, further comprising a second droplet comprising an enzyme required for amplifying the template.

147. The method of claim 144, wherein the filler fluid comprises de-gassed oil.

148. The method of claim 144, wherein the filler fluid comprises hexadecane.

149. The method of claim 144, wherein the filler fluid comprises de-gassed hexadecane.

150. The method of claim 144, wherein the filler fluid comprises hydrocarbon oils.

151. The method of claim 150, wherein the hydrocarbon oils comprises in the range of about 10-20 carbons.

152. The method of claim 144, wherein the filler fluid comprises alkane hydrocarbon oils.

153. The method of claim 152, wherein the alkane hydrocarbon oils comprises in the range of about 10-20 carbons.

154. The method of claim 144, wherein the filler fluid comprises silicone oil

155. The method of claim 144, wherein the filler fluid comprises 2 cSt silicone oil.

156. The method of claim 144, wherein the filler fluid comprises de-gassed silicone oil.

157-165. (canceled)
